(19) United States
(12) Patent Application Publication
(10) Pub. No.: US 2013/0310853 A1
(43) Pub. Date: Nov. 21, 2013

(54) METHOD AND APPARATUS FOR PERCUTANEOUS TREATMENT OF A BLOOD VESSEL

(71) Applicant: ABBOTT CARDIOVASCULAR SYSTEMS INC., Santa Clara, CA (US)

(72) Inventors: Margaret J. Zaugg, Sunnyvale, CA (US); Laveille Kao Voss, Belmont, CA (US); Aaron M. Fortson, Fremont, CA (US); Marc Gianotti, WIESENDANGEN (CH); David J. Milazzo, Santa Clara, CA (US)

(73) Assignee: ABBOTT CARDIOVASCULAR SYSTEMS INC., Santa Clara, CA (US)

(21) Appl. No.: 13/837,801

(22) Filed: Mar. 15, 2013

Related U.S. Application Data
(63) Continuation-in-part of application No. 13/112,618, filed on May 20, 2011.
Application No. 13/112,618 is a continuation-in-part of application No. 12/684,470, filed on Jan. 8, 2010.
Application No. 12/684,470 is a continuation-in-part of application No. 13/112,631, filed on May 20, 2011.
Application No. 13/112,631 is a continuation-in-part of application No. 12/684,470, filed on Jan. 8, 2010.
(60) Provisional application No. 61/143,751, filed on Jan. 9, 2009.
(60) Provisional application No. 61/759,693, filed on Feb. 1, 2013.

(51) Int. Cl. A61B 17/00; A61B 17/04

(52) U.S. Cl. CPC A61B 17/0057; A61B 17/0469; A61B 17/0401
USPC 606/144; 606/232

(57) ABSTRACT

Method and apparatus for percutaneous repair of an affected area in a blood vessel.

### CROSS-REFERENCE TO RELATED APPLICATIONS

This application is a continuation-in-part of U.S. patent application Ser. No. 13/112,618, titled, CLOSURE DEVICES AND METHODS, filed May 20, 2011, which is a continuation-in-part of U.S. patent application Ser. No. 12/684,470, titled CLOSURE DEVICES, SYSTEMS, AND METHODS, filed Jan. 8, 2010, which claims the benefit of U.S. Provisional Application No. 61/143,751, titled VESSEL CLOSURE DEVICES AND METHODS, filed Jan. 9, 2009, which are incorporated herein by reference in their entireties. This application is a continuation-in-part of U.S. patent application Ser. No. 13/112,631, titled, VESSEL CLOSURE DEVICES, SYSTEMS, AND METHODS, filed May 20, 2011, which is a continuation-in-part of U.S. patent application Ser. No. 12/684,470, titled CLOSURE DEVICES, SYSTEMS, AND METHODS, filed Jan. 8, 2010, which claims the benefit of U.S. Provisional Application No. 61/143,751, titled VESSEL CLOSURE DEVICES AND METHODS, filed Jan. 9, 2009, which are incorporated herein by reference in their entireties. This application claims the benefit of U.S. Provisional Application No. 61/759,693, titled METHOD AND APPARATUS FOR PERCUTANEOUS TREATMENT OF A BLOOD VESSEL, filed Feb. 1, 2013, which is incorporated herein by reference in its entirety.

### TECHNICAL FIELD

The disclosed subject matter relates generally to methods and apparatus for the percutaneous treatment of arterial and venous aneurysm, such as abdominal aortic aneurysm, suturing of tissue in various applications such as closure of arterial and venous puncture sites, suturing a graft anastomosis to an aperture in a vessel wall or other types of tissue, and the like, in which no surgical cut down is required.

### BACKGROUND

A number of diagnostic and interventional vascular procedures are now performed transluminally, where a catheter is introduced to the vascular system at a convenient access location and guided through the vascular system to a target location using established techniques. Such procedures require vascular access which is usually established using the well known Seldinger technique, as described, for example, in William Grossman's “Cardiac Catheterization and Angiography,” 3rd Ed., Lea and Febiger, Philadelphia, 1986, incorporated herein by reference.

When vascular access is no longer required, the introducer sheath must be removed and bleeding at the puncture site stopped. One common approach to attempt providing hemostasis (the cessation of bleeding) is to apply external force near and upstream from the puncture site, typically by manual or “digital” compression. This approach suffers from a number of disadvantages. It is time-consuming, frequently requiring one-half hour or more of compression before hemostasis is assured. This procedure is uncomfortable for the patient and frequently requires administering analgesics to be tolerable. Moreover, the application of excessive pressure can at times totally occlude the underlying blood vessel, resulting in ischemia and/or thrombosis. Following manual compression the patient is required to remain recumbent for at least six and at times as long as eighteen hours under close observation to assure continued hemostasis. During this time renewed bleeding may occur resulting in bleeding through the tract, hematoma and/or pseudoaneurism formation as well as arteriovenous fistula formation. These complications may require blood transfusion and/or surgical intervention. The incidence of these complications increases when the sheath size is increased and when the patient is anti-coagulated. It is clear that the standard technique for arterial closure can be risky, and is expensive and onerous to the patient. While the risk of such conditions can be reduced by using highly trained individuals, such use is both expensive and inefficient.

To overcome the problems associated with manual compression, the use of bioabsorbable fasteners to stop bleeding has been proposed by several groups. Generally, these approaches rely on the placement of a thrombogenic and bioabsorbable material, such as collagen, at the superficial arterial wall over the puncture site. While potentially effective, this approach suffers from a number of problems. It can be difficult to properly locate the interface of the overlying tissue and the adventitial surface of the blood vessel, and locating the fastener too far from that surface can result in failure to provide hemostasis and subsequent hematoma and/or pseudo aneurism formation. Conversely, if the fastener intrudes into the arterial lumen, intravascular clots and/or collagen pieces with thrombus attached can form and embolize downstream causing vascular occlusion. Also, thrombus formation on the surface of a fastener protruding into the lumen can cause a stenosis which can obstruct normal blood flow. Other possible complications include infection as well as adverse reactions to the collagen implant.

Catheters are also used to treat heart disease which is a major medical ailment wherein arteries become narrowed or blocked with a build-up of atherosclerotic plaque or clot which reduces flow to tissues downstream or “distal” to the blockage. When this flow reduction becomes significant, a patient's quality of life may be significantly reduced. In fact, heart disease patients often die when critical arteries, such as the coronary arteries, become significantly blocked.

However, technology has been developed to open some blocked arteries in the treatment of heart disease. For example, balloon angioplasty has become a well accepted treatment wherein a balloon is inflated within the narrowed vessel to stretch or otherwise deform the blockage into a larger lumen. Attentively, the blockage can even be removed, such as in a procedure known as atherectomy. In general, these treatments use percutaneous catheters which are inserted into the patients' vessels at a peripheral artery or vein puncture site and guided to the internal blockage site via x-ray visualization. The blockage is then treated remotely by use of hydraulic pressure in the case of balloon angioplasty, or by other actuating means to cause remote cutting or ablation of the blockage in the case of atherectomy.

### Coronary Artery Bypass Graft Surgery (“CABG”)

In the alternative to using catheters to treat heart disease, or when such catheterizations are contraindicated, some blocked vessels can be treated with coronary artery bypass graft surgery (“CABG”). In conventional CABG techniques, a tubular graft is affixed to a port or aperture in an artery wall distally of the blockage. When the opposite end of the tube is in fluid communication with a pressurized arterial blood supply, such as the aorta, the tubular graft provides a conduit for flow into the vessel lumen distally of the blockage.

Conventional CABG surgery is generally initiated by directly exposing the heart to the surgeon. This is accomplished by opening the patient's chest using known sternotomy and retraction techniques that cut the sternum and spread the rib cage open. Then, one or both lungs are usually deflated and the patient is connected to a respiratory assist machine.

Once the heart is exposed, the patient is connected to a coronary bypass machine so that the blood supply circumvents the heart. In this way, the heart is depressurized so that apertures can be cut into the walls of the vessels for surgical graft attachment. The right atrium (or vena cava) and the aorta each is intubated with cannulas which are connected to an artificial pump and oxygenator. Once these major vessels are cannulated, cardioplegia is delivered to slow or stop the beating motion of the heart. The aorta is then clamped proximally of the aortic bypass cannula, thereby isolating the proximal aortic root from the blood that is being circulated by the bypass machine.

After the heart is isolated from blood pressure, conventional bypass grafting is performed. The required grafts are implanted to feed the coronary arteries distal to the blockage, the clamp is removed from the aorta, the lungs are restored, and the patient is then taken off of the bypass pump.

In one type of CABG method, the bypass grafting is achieved between the aorta and one of the three major coronary arteries or their sub-branches, the left anterior descending artery (LAD), the circumflex artery (CIRC), or the right coronary artery (RCA). In such a case, a saphenous vein is usually taken from the patient's leg and is transplanted as a “homograft” to connect these vessels in the same patient's chest. Artificial grafts have also been disclosed as providing potential utility for this purpose and are herein collectively included in the general discussion of “saphenous veins” as used in CABG procedures.

An alternative CABG method uses the internal mammary artery (IMA) alone or in conjunction with the saphenous vein graft. The IMA is severed at a chosen location and is then connected to an aperture, in a coronary artery.

In either case of using saphenous vein homografts or artificial grafts in CABG surgery, the proximal end of the graft is generally sutured or otherwise is affixed circumferentially to the tissue surrounding an aperture that is punched into the wall of the aorta. In this arrangement, the lumen of the graft communicates with the vessel through the aperture, wherein ideally the aperture approximates the inner diameter of the graft lumen. The opposite, distal end of the graft is sutured to an aperture formed in the wall of the coronary vessel distal to the blockage.

The fluid connections between a graft and a vessel are herein referred to as “anastomoses.” In the instance of CABG, “proximal anastomoses” and “distal anastomoses” are terms used when referring to grafting to the aorta and the coronary artery, respectively. In most CABG procedures using saphenous vein grafts, the distal anastomosis is performed first, followed by the proximal anastomosis.

For the CABG method using the IMA, only one distal anastomosis is formed distal to the arterial blockage. A proximal anastomosis to the aorta is not required as it is in a saphenous vein graft procedure because the IMA's natural arterial blood flow feeds the heart.

In conventional CABG surgery methods such as those just summarized, the timing and technique of the anastomosis procedures are critical factors to procedural success. In fact, it is believed that three critical determinants which affect outcomes of CABG surgery are: (1) time the patient spends on bypass, (2) time the patient spends with a clamped aorta, and (3) the quality of the anastomoses. It is generally believed that a CABG patient's operative and peri-operative morbidity are directly related to how long the patient must be on heart bypass. In fact, it is generally understood that the risk of patient morbidity is believed to rise significantly after a threshold time of one hour on bypass. Perhaps the most prevalent complication arising from prolonged cardiac bypass is the high risk of distal thrombus created by the artificial plumbing. For example, such thrombi can embolize into the neurovasculature and potentially can cause a stroke. In analyzing the timing of individual CABG steps against the backdrop of a patient's critical time on bypass, the time spent anastomosing the grafts to vessels emerges as a controlling factor. The average time for suturing one anastomosis is approximately 7-10 minutes. Furthermore, it is believed that an average CABG procedure involves approximately five anastomoses: two saphenous vein grafts, each with a proximal and a distal anastomosis, and one internal mammary artery having only one distal anastomosis. Therefore, the average time for graft suturing ranges from 35 minutes to 50 minutes—in any case a significant portion of the 60 minute critical threshold to patient morbidity. Closely related to the time spent on bypass is a second CABG success factor related to the extent and time of aortic cross-clamping. It is believed that the inherent crushing force from a cross-clamp across the bridge of the muscular aortic arch may be associated with a high degree of tissue trauma and structural damage. Additionally, hemostasis formed at or adjacent to the cross clamp, perhaps in conjunction with the tissue trauma of clamping, may also be a source of unwanted thrombogenesis.

In addition to the timing of anastomosing grafts and extent and duration of aortic cross-clamping, the quality of interface between the graft and vessel is also believed to be an indicator of procedural success. The accuracy, trauma, and repeatability of suturing, as well as the three-dimensional interface formed between the conduits at the anastomosis site, are significant variables in conventional manual surgical techniques. These variables are believed to significantly affect the short or long-term success of conventional CABG anastomosis procedures.

### Limitations of Conventional CABG Devices & Methods

Both of the critical CABG success indicators summarized above—time on cardiac bypass and quality of anastomosis suturing—are directly affected by inherent limitations in the devices used in conventional CABG procedures. It is believed that improvements to these devices and related methods of use may provide for more rapid and reliable vessel-graft anastomosing. For example, conventional “surgical punches” are devices that cut or “punch” a plug in vessel wall tissue to form an aperture in the wall. In a CABG procedure, the tissue surrounding a punched-out aperture provides the substrate upon which a graft may be sutured to form an anastomosis. One procedural limitation in using conventional surgical punches is that hemostasis can not be maintained at a vessel wall after a plug of tissue is punched out and removed. Therefore, an aperture in an aortic wall during a saphenous vein graft procedure can only be made when that portion of the aorta is cross-clamped, bypassed, and depressurized. Otherwise, the high blood pressure and flow in the aorta would cause significant bleeding during the period from punching the aperture to forming the anastomosis. Because of this limitation in conventional surgical punches, the threshold 60 minute coronary bypass clock begins running before punching the aorta.

The prior art fails to disclose or fulfill the need which exists in the field of medical devices and methods for: suturing tissue by proximally drawing sutures through a tissue layer in the proximity of an aperture; suturing tissue by reversibly advancing needles from one side of a tissue layer to retrieve one or more sutures on the opposite side of the tissue layer; a medical device assembly and method that automatically and repeatably places suture thread through vessel wall tissue surrounding an aperture in the vessel wall in a suture pattern that is useful for anastomosing a tubular graft to the aperture; and a medical device assembly that deploys a suture with one end extending through the tissue that surrounds a aperture in a vessel wall and the opposite suture end extending radially through a tubular graft wall adjacent an open end of the graft, such that a vessel anastomosis may be rapidly and repeatably performed in a CABG procedure even while the vessel is under physiological flow.

The prior art likewise fails to disclose a medical device or method to overcome anatomical challenges associated with a totally endoscopic technique. Among these challenges are calcification of the vessel, groin scarring/recent catheterization, and patient obesity. Prior published experience suggests that significant ipsilateral groin scarring and morbid obesity are suitable exclusionary conditions. Other predictors included larger sheath size and pre-existing groin scarring (odds ratios 1.2 and 8.2, respectively, P<0.05). Groin scarring was a significant predictor of late access-related intervention (odds ratio 49, P<0.001).

In patients with suitable anatomy for an endoluminal stent graft, endovascular abdominal aortic aneurysm repair (“EVAR”) has become an appealing alternative to open surgical repair due to its low perioperative morbidity and mortality. Moreover, device improvements have led to the expansion of the patient population in which EVAR may be offered, either through a wider range of stent graft sizes, improved materials, or smaller catheter based access sheaths.

Currently in the United States, endograft device labeling indicates the introduction of the main device (ranging from a profile of 8 Fr to 26 Fr) through surgical vascular exposure of the ipsilateral common femoral artery (CFA). Endovascular grafting has markedly reduced the invasiveness of the treatment of abdominal aortic aneurysms; it has reduced the length of hospital stays, intensive care requirements and operative morbidity.

It is often desirable to provide additional treatment for the access site when positioning the sutures and closing the opening in the vessel. What is needed is a apparatus for applying coated sutures for administering a medication to the vessel tissue. Moreover, what is needed is an a apparatus for adjustment of the spacing between needles during the positioning of sutures through the vessel tissue.

### SUMMARY

The present invention provides a totally percutaneous method of treating an affected region of a blood vessel, the method including creating an access point in a blood vessel of a patient proximal to the affected site without a surgical cut-down procedure; advancing a guidewire through the access point and traversing the guidewire to a location distal to the affected site; advancing a closure device into the blood vessel; selectively adjusting the spacing of a plurality of needles; advancing the needles through a wall of the blood vessel thereby positioning a suture through the wall of the blood vessel across the access point; removing the closure device from the subject; delivering an medical device to the location of the affected site; and securing the suture to close the access point of the blood vessel.

In some embodiments, the method includes providing two or more needles having a first pre-curved configuration and a second straightened configuration, and wherein selectively adjusting the spacing of the needles comprises withdrawing a sheath surrounding the needles thereby permitting the needles to move from the second configuration to the first configuration. In some embodiments, the spacing of the plurality of needles is adjustable between about 8 Fr and about 24 Fr.

In some embodiments, the blood vessel is a vein. In some embodiments, the suture is coated with a hydrogel, an antibiotic, a pro-coagulent medication, and/or an anti-inflammatory medication.

In some embodiments, advancing the closure device into the blood vessel includes advancing the closure device at an angle of about 45° relative to the blood vessel. In some embodiments, the method further includes dilating the blood vessel prior to insertion of the closure device.

In some embodiments, the closure device includes a shaft having a proximal end and a distal end and defining an axis therebetween; an elongate foot movably mounted to the shaft, the foot supporting the suture; an actuator extending along the shaft distally to the foot, movement of the actuator pivoting the foot from a low profile configuration aligned along the shaft to a deployed configuration extending laterally from the shaft; and a needle advanceable from the shaft through the tissue and to the deployed foot.

In some embodiments, the needle has a proximal end and a distal end suitable for forming a first needle path through the vessel wall; and a first fitting attached to the suture, the first fitting securingly engaging the first needle when the first needle advances through the vessel wall so that the first fitting and at least a portion of the suture can be withdrawn proximally along the first needle path by the first needle.

In some embodiments, the foot has a first end and a second end, wherein a plurality of needles are extendable from the shaft to the ends of the foot.

In some embodiments, the suture is a monofilament suture.

In some embodiments, positioning a suture through a wall of the blood vessel across the access point includes positioning the suture through the wall of the blood vessel at first angle with respect to the longitudinal axis of the patient; and further comprising after removing the first closure device from the subject, advancing a second closure device into the blood vessel; positioning a second suture through a wall of the blood vessel across the access point at a second angle with respect to the longitudinal axis of the patient; removing the second closure device from the subject.

In some embodiments, the first angle is 60 degrees in a first direction from the longitudinal axis of the patient. In some embodiments, the second angle is 60 degrees in a second direction from the longitudinal axis of the patient.

A totally percutaneous method of treating an affected region of a blood vessel is provided, the method including creating an access point in a blood vessel of a patient proximal to the affected site without a surgical cut-down procedure; advancing a guidewire through the access point and traversing the guidewire to a location distal to the affected site; advancing a closure device into the blood vessel; positioning an anchor carrying a suture therewith through a wall of the blood vessel across the access point; deploying the anchor with respect to the wall of the blood vessel; removing the first closure device from the subject; delivering a medical device to the location of the affected site; and securing the suture to close the access point of the blood vessel.

In some embodiments, the blood vessel is a vein.

In some embodiments, the method further includes providing an anchor defining a longitudinal axis, a central opening for retaining a suture therein, and a notch extending from the central opening allowing the suture to pass from the central opening through the notch at an angle with respect to the longitudinal axis, and wherein deploying the anchor comprises withdrawing the suture from the vessel such that the anchor engages the wall of the vessel and the suture extends from the anchor at a angle with respect to the longitudinal axis of the anchor.

In some embodiments, the method further includes defining a plurality of vanes on the suture, and wherein deploying the anchor comprises engaging the vanes with the notch of the anchor and the vessel wall. In some embodiments, the method further includes defining a plurality of ridges on the notch for engaging the suture, and wherein deploying the anchor comprises engaging the suture with the ridges of the notch. In some embodiments, the anchor is fabricated from magnesium.

In some embodiments, the method further includes providing an anchor defining a longitudinal axis, a central opening for retaining a suture therein, and a plurality of longitudinal notches defining a plurality of grappler arms, and wherein deploying the anchor comprises withdrawing the suture from the vessel such that the plurality of grappler arms are displaced radially outwardly from the longitudinal axis and engage the wall of the vessel.

In some embodiments, the method further includes providing an anchor comprising a resilient strip having a first configuration defining a coil and a second straightened configuration, and wherein advancing a closure device into the blood vessel comprises restraining the resilient strip in the first configuration within an introducer.

In some embodiments, the method further includes positioning the anchor carrying a suture therewith through a wall of the blood vessel across the access point comprises advancing the introducer through the wall of the blood vessel with the resilient strip disposed therein.

In some embodiments, deploying the anchor with respect to the wall of the blood vessel includes advancing the resilient strip with respect to the introducer into the blood vessel, thereby allowing the resilient strip to return to the first configuration.

An apparatus for the percutaneous closure of an opening created in a blood vessel is provided including a shaft defining a bore therethrough for guidewire access; two or more needles defining a first configuration defining a curved configuration and a second straightened configuration; a sheath surrounding the needles and advanceable from a first position in which the needles are restrained in the second configuration and a second position in which the needles are permitted to return to the first configuration; and one or more sutures attached to an end portion of the needles.

In some embodiments, the sheath is sized to fit through an opening in the blood vessel of about 8 Fr. In some embodiments, the method further includes the sheath is sized to fit through an opening in the blood vessel of about 24 Fr. In some embodiments, the needle is fabricated from a shape memory alloy. In some embodiments, the needle is fabricated from nitinol. In some embodiments, the shaft and the sheath are flexible.

An apparatus for the percutaneous closure of an opening created in a blood vessel is provided including an anchor defining a longitudinal axis, a central opening for retaining a suture therein, and a notch extending from the central opening allowing the suture to pass from the central opening through the notch at an angle with respect to the longitudinal axis, a suture partially positioned within the central opening and retained therein, the suture partially extending from the central opening, an introducer for advancing through the wall of the vessel and accommodating the anchor therein.

In some embodiments, the suture defines a plurality of vanes thereon for engagement with the notch of the anchor and the vessel wall. In some embodiments, the notch defines a plurality of ridges for engaging the suture. In some embodiments, the anchor is fabricated from magnesium.

An apparatus for the percutaneous closure of an opening created in a blood vessel is provided including an anchor defining a longitudinal axis, a central opening for retaining a suture therein, and a plurality of longitudinal notches defining a plurality of grappler arms for engagement with the wall of the vessel; a suture partially positioned within the central opening and retained therein, the suture partially extending from the central opening; an introducer for advancing through the wall of the vessel and accommodating the anchor therein.

An apparatus for the percutaneous closure of an opening created in a blood vessel is provided including an anchor comprising a resilient strip having a first configuration defining a coil and a second straightened configuration, and wherein advancing a closure device into the blood vessel comprises restraining the resilient strip in the first configuration within an introducer, a suture partially positioned within the central opening and retained therein, the suture partially extending from the central opening, and an introducer for advancing through the wall of the vessel and accommodating the anchor therein in the first configuration and for deploying the anchor from therefrom in the second configuration.

### BRIEF DESCRIPTION OF THE DRAWINGS

In the drawings, which comprise a portion of this disclosure but are not to scale:

FIG. 1 is a perspective of a percutaneous blood vessel closure device according the principles of the present invention;

FIG. 2 illustrates the vessel closure device of FIG. 1 in which an elongate foot is shown in a deployed position;

FIGS. 2A-C illustrate actuation of a foot and advancement of needles from a shaft to the articulated foot in a probe similar to the probe of FIG. 1;

FIG. 3A is a detailed view showing the foot of the vessel closure device of FIG. 1 in a parked position prior to deployment;

FIG. 3B is a detailed view showing the foot of the vessel closure device of FIG. 1 in a deployed position;

FIGS. 4 and 4A are perspective views illustrating a suture attachment cuff and an associated barbed needle for use in the vessel closure device of FIG. 1;

FIG. 5 is a cross-sectional view showing the barbed needles securingly engaging the suture cuffs of the deployed foot;

FIGS. 6A-C illustrate one embodiment of a deployable foot, in which the foot slides and pivots when drawn proximally by a tension member;

FIG. 7 illustrates the suture cuff positioned within a needle receptacle, and also shows how the suture is releasably secured within a slot extending radially from the needle receptacle;

FIGS. 8A-C illustrate an alternative foot articulation mechanism in which lateral slots on the foot receive pins from the shaft to allow the foot to pivot and slide axially;

FIGS. 9A and B illustrate a still further alternative foot actuation mechanism in which the foot slides axially within a slot;

FIGS. 9C and D illustrate a further foot actuation mechanism in which relative movement between the sides of a two-part shaft actuates the foot;

FIGS. 10A-D illustrate alternative structures and techniques for avoiding entanglement of the needle with the suture;

FIGS. 11A-E illustrate an alternative closure system and method for its use in which a first needle advances the suture to the foot, while a second needle engages and withdraws both the first and second suture cuffs, a flexible filament connecting the suture cuffs, and at least a portion of the suture from within the blood vessel so as to complete a pre-tied knot;

FIGS. 12A and B illustrate an alternative probe having two pairs of needles and a foot with four needle receptacles so as to form two loops of suture across a puncture of a blood vessel;

FIGS. 13A-G illustrate a method for use of a suture system so as to effect hemostasis of a blood vessel puncture through a tissue tract;

FIGS. 14A and 14B are enlarged partial side views of a suturing device in accordance with one embodiment of the present invention;

FIGS. 15A through 15F are enlarged cross-sectional views of the embodiment of the suturing device of FIGS. 14A and 14B;

FIGS. 16A and 16B are schematic views of a suture bight having a pre-tied knot in accordance with one embodiment of the present invention;

FIGS. 17A through 17D show enlarged partial cross-sectional views of an embodiment of the suturing device in accordance with the invention, in which one embodiment of a penetrator tip and cuff engagement, penetrator tip disengagement, and cuff ejection sequence is illustrated;

FIG. 18A is an enlarged partial cross-sectional view of an embodiment of a foot in accordance with the present invention, showing the link routing through the suture bearing surfaces of the foot;

FIG. 18B is an enlarged partial cross-sectional view of an embodiment of a device in accordance with the present invention, showing the link routing through a suture-bearing surface located distal to the foot;

FIGS. 19A and 19B are enlarged partial cross-sectional views of an embodiment of a foot in accordance with the present invention, showing an alternate penetrator tip and cuff engagement, penetrator tip disengagement, and cuff ejection sequence;

FIGS. 20A through 20C are enlarged partial cross-sectional views of an embodiment of a foot in accordance with the present invention, showing an alternate penetrator tip and cuff engagement, penetrator tip disengagement, and cuff ejection sequence;

FIG. 21 is an enlarged perspective view of an embodiment of the pre-tied knot in accordance with the present invention;

FIGS. 22A through 22C show an alternate embodiment of a foot in accordance with the invention;

FIGS. 23A through 23C show another alternate embodiment of a foot in accordance with the invention;

FIGS. 24A and 24B are perspective views of an alternative embodiment of a penetrator tip in accordance with the invention;

FIGS. 25A through 25C are schematic views of an alternate embodiment of a vessel closure device in accordance with the present invention;

FIGS. 26A through 26D are schematic views of alternate embodiments of a vessel closure device in accordance with the invention;

FIG. 27 shows a schematic view of one embodiment of a link and cuff assembly in accordance with the invention;

FIG. 28 is a perspective view of an embodiment of a suturing device constructed in accordance with the principles of the present invention;

FIG. 29A is a detail view of the distal end of the guide body of the suturing device of FIG. 28, shown with the needles retracted fully within the guide body;

FIG. 29B is a view similar to FIG. 29A, except that the needles have been partially drawn back into the guide body;

FIG. 30 is a cross-sectional view of the device of FIGS. 29A and 29B, taken along line 36-36 of FIG. 2B;

FIGS. 31-34 illustrate the method of the present invention using the suturing device 30 of FIG. 28;

FIG. 35 illustrates the X-pattern of the tied suture applied by the suturing device of FIG. 28;

FIG. 36 illustrates an apparatus for advancing a suture knot in accordance with embodiments of the disclosed subject matter;

FIG. 37 illustrates an apparatus for advancing a suture knot and/or trimming a suture in accordance with embodiments of the disclosed subject matter;

FIGS. 38-39 illustrate positioning of sutures on a vessel in accordance with embodiments of the disclosed subject matter;

FIGS. 40-44 illustrate suture configurations in accordance with embodiments of the disclosed subject matter;

FIG. 45 illustrates a pair of needles in accordance with an embodiment of the disclosed subject matter;

FIG. 45A illustrates a cross-sectional view of an apparatus for use with the needles of FIG. 45, in accordance with an embodiment of the disclosed subject matter;

FIG. 46 illustrates a pair of needles illustrated in FIG. 45 in a first configuration with respect to a sheath in accordance with an embodiment of the disclosed subject matter;

FIG. 47 illustrates a pair of needles illustrated in FIG. 45 in a second configuration with respect to a sheath in accordance with an embodiment of the disclosed subject matter;

FIG. 48 illustrates a pair of needles illustrated in FIG. 45 in a third configuration with respect to a sheath in accordance with an embodiment of the disclosed subject matter;

FIG. 49 is a cross-sectional view showing the needles in a first configuration securingly engaging the suture cuffs of the deployed foot in accordance with an embodiment of the disclosed subject matter;

FIG. 50 is a cross-sectional view showing the needles in a second configuration securingly engaging the suture cuffs of the deployed foot in accordance with an embodiment of the disclosed subject matter;

FIG. 51 is a detail view of the distal end of the guide body of a suturing device, shown with the needles partially extended form the guide body relative to a sheath in accordance with an embodiment of the disclosed subject matter;

FIG. 52 is a cross-sectional view showing catheter and guide wire inserted within a vessel in accordance with an embodiment of the disclosed subject matter;

FIG. 53 is a cross-sectional view showing the removal of the guide wire in accordance with an embodiment of the disclosed subject matter;

FIG. 54 is a cross-sectional view showing the deployment of an anchor in accordance with an embodiment of the disclosed subject matter;

FIG. 55 is a cross-sectional view showing the engagement of the anchor with the vessel wall in accordance with an embodiment of the disclosed subject matter;

FIG. 56 is a cross-sectional view showing the deployment of a cap in accordance with an embodiment of the disclosed subject matter;

FIG. 57 is a cross-sectional view showing the further deployment of the cap in accordance with an embodiment of the disclosed subject matter;

FIG. 58 is a cross-sectional view showing the further deployment of the cap in accordance with an embodiment of the disclosed subject matter;

FIG. 59 is a cross-sectional view showing the deployment of a pair of anchors in accordance with an embodiment of the disclosed subject matter;

FIG. 60 is a perspective view illustrating a further embodiment of an anchor in accordance with an embodiment of the disclosed subject matter;

FIG. 61 is a perspective view illustrating another embodiment of an anchor in accordance with an embodiment of the disclosed subject matter;

FIG. 62 is a cross-sectional view illustrating the deployment of the anchor of FIG. 61 in accordance with an embodiment of the disclosed subject matter;

FIG. 63 is a perspective view illustrating another embodiment of an anchor in accordance with an embodiment of the disclosed subject matter;

FIG. 64 is a cross-sectional view illustrating the deployment of the anchor of FIG. 63 in accordance with an embodiment of the disclosed subject matter;

FIG. 65 is a side view illustrating another embodiment of an anchor in accordance with an embodiment of the disclosed subject matter;

FIG. 66 is a cross-sectional view of the anchor in a straightened configuration in accordance with an embodiment of the disclosed subject matter;

FIG. 67 is a cross-sectional view of the anchor in a partially deployed configuration in accordance with an embodiment of the disclosed subject matter; and

FIG. 68 is a cross-sectional view showing the deployment of a pair of anchors of FIGS. 65-67 in accordance with an embodiment of the disclosed subject matter.

### DETAILED DESCRIPTION

The term “distal” as used herein, is a broad term and is used in its ordinary sense, including, without limitation, as in the direction of the patient, or away from a user of a device, or in a downstream direction relative to a forward flow of blood. In the context of a medical device intervention with or through a vessel wall, “distal” herein refers to the interior or the lumen side of the vessel wall.

The term “proximal” as used herein, is a broad term and is used in its ordinary sense, including, without limitation, as away from the patient, or toward the user, or in an upstream direction relative to a forward flow of blood. In the context of a medical device intervention with or through a vessel wall, “proximal” herein refers to the exterior or outer side of the vessel wall.

The term “oblong” as used herein, is a broad term and is used in its ordinary sense, including, without limitation, oval, elliptical, or otherwise having a generally rounded shape that is not perfectly circular. In particular, the term describes the shape of a tubular graft end cut at an acute angle relative to the plane perpendicular to the tissue walls defining the graft.

The term “hemostasis” as used herein, is a broad term and is used in its ordinary sense, including, without limitation, the arrest of bleeding or substantially blocking flow of blood outwardly from a vessel lumen while the vessel lumen is pressurized or sustaining physiological blood flow. This amount of blockage or occlusion to flow is further defined such that the blood loss which is experienced is less than an amount which would affect procedural methods or outcomes according to a physician user of a device of ordinary skill in the art. In other words, “hemostasis” is not intended to mean only “total hemostasis” such that there is a total lack of blood loss. Rather, the term is used to also mean “procedural hemostasis” as a relative term in its use among physicians of ordinary skill.

Similarly, the terms “occlusion,” “occlude,” “blockage,” “block . . . plugging”, “block,” or variations thereof are all terms which are herein intended to have a procedurally relevant definition in the context of their use. For instance, an aperture is “occluded” although there is some measurable flow therethrough, but that flow is so low such that the intended procedural benefit of occlusion is at least partially achieved. Certainly, such terms also properly include within their scope a “total effect” definition, as well.

The term “perfusion” as used herein, is a broad term and is used in its ordinary sense, including, without limitation, the flow of blood or other unit of perfusate (the fluid used for perfusion) per unit volume of tissue. Physiological perfusion refers to the amount of blood flow present when the body is functioning normally. For example, physiological perfusion usually prevents clinically significant ST elevations which is one of the most sensitive indicators of inadequate perfusion. Adequate perfusion refers to the amount of blood flow that avoids the clinical requirement of transfusing the patient or that is needed to prevent tissue necrosis distal to the aperture in the blood vessel.

The term “suturing” as used herein, is a broad term and is used in its ordinary sense, including, without limitation, the process of joining two surfaces or edges together with a fasten r so as to close an aperture, opening, or wound or join tissues. The fastener is usually a suture such as a thread of material (either polymeric or natural), gut, wire or the like. The term “fastener” as used herein, is a broad term and is used in its ordinary sense, including, without limitation, also includes clamps, studs, hasps, catches, hooks, rivets, staples, snaps, stitches, VELCROC, buttons, and other coupling members.

The term “PEVAR” as used herein, is a broad term and is used in its ordinary sense, including, without limitation, endovascular aneurysm repair using a bilateral access approach.

The term “pre-close” as used herein, is a broad term and is used in its ordinary sense, including, without limitation, the placement of the sutures in a blood vessel, e.g., femoral artery, before the arteriotomy is enlarged by an endovascular sheath.

The term “surgical cut-down” as used herein, is a broad term and is used in its ordinary sense, including, without limitation, the surgical exposure of the vessel for device introduction.

The term “procedural technical success” or “procedural success” as used herein, is a broad term and is used in its ordinary sense, including, without limitation, successful vascular access, delivery of graft introducer catheter, e.g., IntuiTrak device delivery, deployment and catheter removal without vascular exposure.

The term “vascular complication” as used herein, is a broad term and is used in its ordinary sense, including, without limitation, any of the following that requires medical or surgical intervention: arteriovenous fistula, femoral neuropathy, hematoma requiring drainage or other surgical intervention, infection, lymphocele, hemorrhage, vascular (iliac or femoral artery) injury or pseudoaneurysm requiring surgical repair; stenosis, distal emboli or thrombosis/occlusion of a peripheral artery or stent graft limb, and the like.

The term “major adverse event” as used herein, is a broad term and is used in its ordinary sense, including, without limitation, all-cause death, conversion to open repair, aneurysm or blood vessel rupture, secondary intervention for type I or III endoleak; bowel ischemia; cardiac morbidity, neurological complications, renal failure, respiratory complications, and the like.

The term “time to hemostasis is defined as the elapsed time from sheath removal to first observed cessation of CFA bleeding (excluding cutaneous or subcutaneous oozing).

The term “device implant time” as used herein, is a broad term and is used in its ordinary sense, including, without limitation, the elapsed time between IntuiTrak sheath introduction and removal.

The term “procedure time” as used herein, is a broad term and is used in its ordinary sense, including, without limitation, the elapsed time from the first break of skin to final closure (i.e., skin to skin time).

The term “time to ambulation” as used herein, is a broad term and is used in its ordinary sense, including, without limitation, the elapsed time from sheath removal to the time when the patient stands and walks at least 20 feet without re-bleeding.

The disclosures of U.S. Pat. Nos. 5,304,184; 5,476,469; 5,720,757; 5,746,755; 5,797,929; 5,810,850; 6,117,145; 6,132,440; 6,136,010; 6,190,396; 6,348,059; 6,358,258; 6,558,399; 6,730,102; 6,746,457; 6,964,668; 7,001,400; 7,029,487; 7,048,747; 7,060,078; 7,094,246; 7,147,646; 7,201,762; 7,235,087; and 7,445,626; and U.S. Publication Nos. 2011/0288563; 2011/0218568; and 2010/0185234 are incorporated by reference herein for all purposes.

A suturing device, which delivers a pre-tied knot to an incision, is disclosed. As an overview, a suturing device in accordance with the present invention includes a first penetrator having a pre-tied knot disposed thereabout and a second penetrator having suture disposed thereon. During operation of the suturing device, the first penetrator and the second penetrator penetrate the tissue about a periphery of an incision in a body lumen. Upon penetration, a penetrator tip releasably engaged with the first penetrator couples with a foot of the suturing device. As the first and second penetrators retract from the body lumen, the penetrator tip and the suture coupled with the penetrator tip retract through a penetration formed in the body lumen by the first penetrator. As will be discussed in greater detail with reference to the accompanying Figures, as the suture refracts, the pre-tied knot receives the suture, forming a knot for suturing the incision in the body lumen.

Referring now to FIG. 1, a vessel closure device 10 generally has a shaft 12 having a proximal end 14 and a distal end 16. A proximal housing 18 supports a needle actuation handle 20. A flexible, atraumatic monorail guidebody 22 extends distally of distal end 16 of shaft 12.

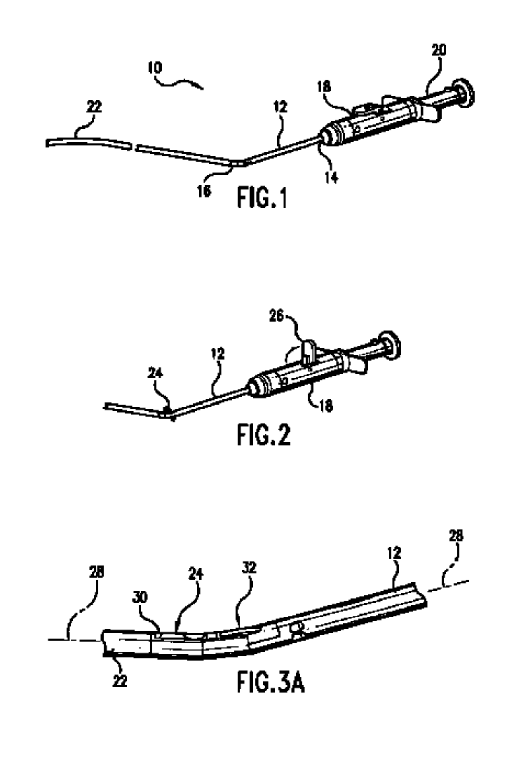

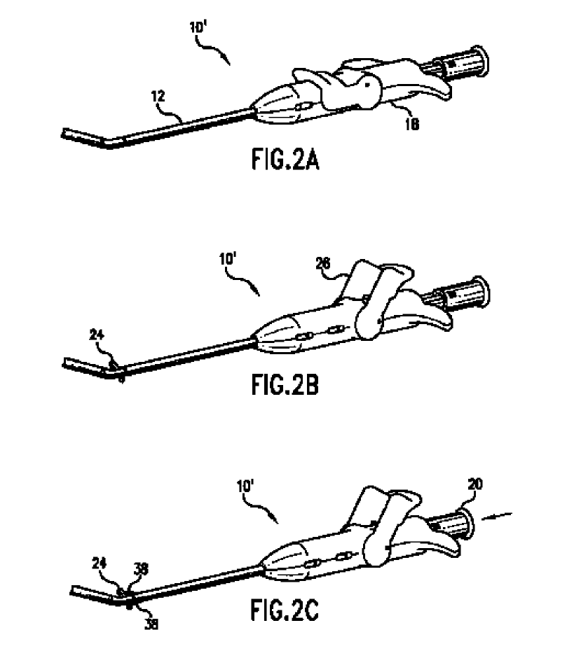

As can be seen with reference to FIG. 2, a foot 24 is articulatably mounted near the distal end of shaft 12. Foot 24 moves between a low profile configuration, in which the foot is substantially aligned along an axis of shaft 12 (as illustrated in FIG. 1), to a deployed position, in which the foot extends laterally from the shaft, upon actuation of a foot actuation handle 26 disposed on proximal housing 18.

FIGS. 2A through C illustrate the structure and actuation of foot 24 of a preferred probe 10′ having a modified proximal housing, and also show how needles 38 can be advanced distally from shaft 12 to the foot by depressing needle actuation handle 20.

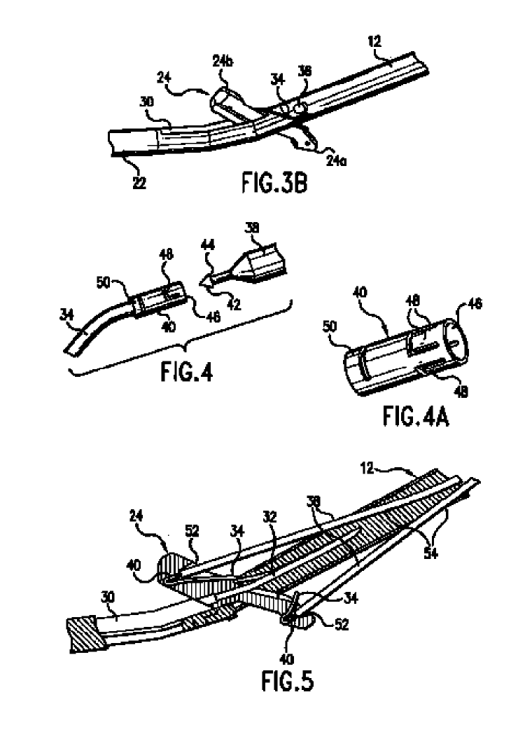

Actuation of foot 24 is illustrated more clearly in FIGS. 3A and B. In the parked position illustrated in FIG. 3A, foot 24 extends substantially along axis 28 of shaft 12. Note that the axis of the shaft need not be straight, as the shaft may curve somewhat, particularly adjacent the foot. In the exemplary embodiment, foot 24 is substantially disposed within a foot receptacle 30 of shaft 12 so as to minimize the cross-section of the device adjacent the foot prior to deployment. Advantageously, prior to deployment of the foot, device 10 can have a cross-section adjacent foot 24 of about 7 Fr or less, ideally having a cross-section of about 6 Fr or less for the entire device distally of the proximal end 14 of shaft 12.

Actuation of foot handle 26 slides a foot actuation wire 32 proximally, pulling foot 24 from a parked position to the deployed position illustrated in FIG. 3B. Once deployed, a first end 24a and a second end 24b of foot 24 extend laterally from the shaft. Suture 34 here comprises a continuous filament with ends disposed in needle receptacles adjacent each end of the foot. An intermediate portion of suture 34 may extend proximally along a suture lumen of shaft 12 to and/or beyond proximal housing 18. Alternatively, in preferred probe 10′, the length of suture between the ends may extend distally within flexible guidebody 22, preferably in a dedicated lumen (separate from the monorail guidewire lumen). In still further alternatives described below, a short length of suture or some other flexible filament may extend substantially directly between the needle receptacles.

Shaft 12 also includes a foot position verification lumen that extends distally from a position verification port 36 to a position indicator at housing 18. When the foot is properly positioned within the blood vessel, blood pressure will cause blood to flow proximally through the indicator lumen to the indicator. The indicator may optionally comprise a blood exit port, a clear receptacle in which blood is visible, or the like. In the exemplary embodiment, the indicator of handle 18 comprises a length of clear tubing extending from housing 18 (not shown) in which the blood is clearly visible. It should be understood that a wide variety of alternative position verifications sensors might be used, including electrical pressure sensors, electrolytic fluid detectors, or the like.

The structures used in positioning a loop of suture across the puncture can be understood with reference to FIGS. 4, 4A, and 5. In general terms, needles 38 extend from shaft 12 into secured engagement with fittings 40 attached to sutures 34. More specifically, needles 38 include a barbed end 42 defining a recessed engagement surface 44. Fittings 40 are roughly cylindrical structures having an axial channel 46 that receives barbed end 44 of needle 38 therein. A first slot is cut in fitting 44 so as to define at least one tab 48. Tabs 48 can be resiliently biased inward into channel 46. As needle 38 advances into fitting 40, barbed end 42 resiliently displaces tab 48 clear of channel 46 so as to allow the barbed end to pass axially into the fitting. Once barbed end 42 is disposed axially beyond tab 48, the tab resiliently flexes back into the channel, capturing needle 38 by engagement between the tab and recessed surface 44. As each tab can hold the fitting in place on the needle, the use of more than one tab increases the reliability of the system. Ideally, three tabs are provided, as illustrated in FIG. 4A.

To facilitate attachment of fitting 40 to suture 34, a second slot cut in the tubular fitting structure defines a suture attachment collar 50. Optionally, collar 50 may be crimped about suture 34 to mechanically affix the suture to fitting 40. In addition and/or instead of mechanical crimping, suture 34 may be bonded to fitting 40 using an adhesive, heat, fasteners, knots, or the like.

Fitting 40 is quite small in size, and is generally configured to facilitate withdrawing the fitting (and the attached suture) along with needle 38 axially through the vessel wall along the needle path. Needle 38 will generally have a cross-sectional width of between about 0.010 inches and 0.020 inches. Barb 42 will extend laterally so as to define an engagement surface 44 having a protruding length of between about 0.002 inches and 0.005 inches. Fitting 40 will preferably have a cross-sectional size roughly corresponding to or only slightly larger than needle 38. Fitting 40 will typically have an outer lateral width of between about 0.014 inches and 0.025 inches, and an axial length of between about 0.035 inches and 0.050 inches. Channel 46 will be sized to receive at least a portion of needle 38, and will generally have a width of between about 0.010 inches and 0.020 inches. Suture 34 will preferably extend axially opposite the open end of channel 46 so as to minimize drag when the suture is drawn proximally along the needle path. In the exemplary embodiment, needle 38 has a diameter of about 0.020 inches, while the fitting comprises a tube having an outer diameter of about 0.020 inches, an inner diameter of about 0.016 inches, and an overall length of about 0.047 inches. The fitting will typically comprise a resilient material, preferably comprising a metal, and in the exemplary embodiment, comprising stainless steel.

Needles 38 typically have a length of between about 5.0 inches and 6.0 inches, and will preferably be sufficiently stiff to be advanced in compression through the vessel wall (and adjacent tissues) for up to 0.5 inches when supported in cantilever. Nonetheless, the needles will ideally be flexible enough to be laterally deflected within shaft 12, as can be understood with reference to FIG. 5. Needles 38 generally comprise a high strength metal, ideally comprising stainless steel. Fittings 40 will also preferably comprise a flexible material to allow tab 48 to flex out of the way of barbed end 42, and to resiliently rebound and engage recessed surface 44. In the exemplary embodiment, barbed end 42 has a diameter of about 0.015 inches, with the diameter of the needle decreasing to about 0.008 inches proximally of the barb so as to define the recessed engagement surface.

As was generally described above, foot 24 includes needle receptacles 52 adjacent the ends of the foot. A fitting 40 (with an associated end of suture 34) is disposed within each needle receptacle, and a surface of the receptacle tapers proximally and outwardly so as to guide the advancing needles 38 into engagement with fittings 40 when foot 24 is in the deployed position. As fittings 40 (and associated portions of suture 34) are releasably supported in the foot, needles 38 can be withdrawn proximally so as to draw the fittings and suture ends from the foot proximally into (and optionally through) shaft 12. The needle receptacles of the exemplary embodiment taper outward at an angle between 20 and 35 degrees from the centerline of fitting 40, and the fitting is held in a recess having a diameter of about 0.0230 inches and a length of about 0.042 inches. A lateral opening or window through the side of foot to the fitting recess may be provided to facilitate needle and/or cuff positioning during assembly of the probe, and a protruding collar near the proximal end of the fitting recess may help keep the fitting in position.

FIG. 5 also illustrates the lateral deflection of needles 38 by needle guides 54 of shaft 12. This lateral deflection of the needles allows the use of a small diameter shaft, while still encompassing sufficient tissue within the suture loop on opposite sides of the puncture so as to effect hemostasis when the suture looped is tightened and secured. In the exemplary embodiment, shaft 12 comprises an outer casing of a biocompatible material such as stainless steel, carbon fiber, nylon, another suitable polymer, or the like. Needle guides 54 may be defined at least in part as lumens formed within the casing of a polymeric material such as nylon or the like. In some embodiments, shaft 12 may comprise a carbon fiber filled nylon, or carbon fiber filled with an alternative material.

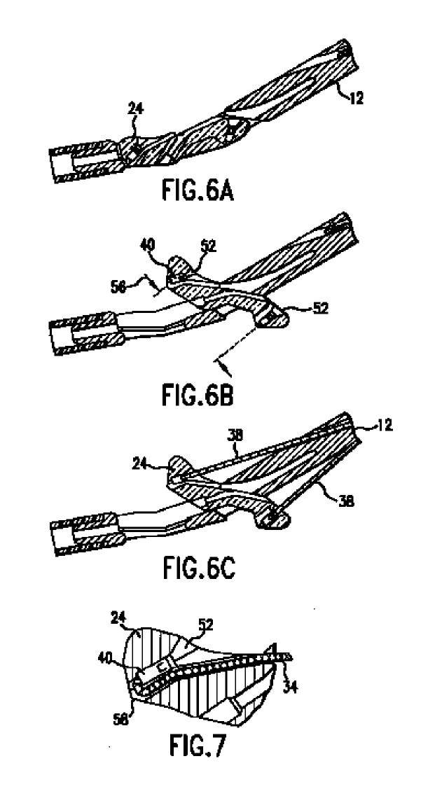

One example of a suitable structure and articulation motion for foot 24 is illustrated in FIGS. 6A and B. Foot actuation wire 32 (see FIG. 3A) rides in a lumen of shaft 12, and draws foot 24 from a parked position (shown in FIG. 6A) to a deployed position (shown in FIG. 6B) through a combination of sliding and pivoting of the foot. The foot remains supported throughout its range of motion by arms disposed laterally on either side of the foot, the arms defining (at least in part) foot receptacle 30. Once foot 24 is deployed, needle receptacles 52 and/or the fittings disposed therein will preferably define a lateral suturing width 56 in a range from about 0.260 inches to about 0.300 inches. Foot 24 may be machined or cast from a polymer or metal, but will preferably comprise a polymer such as carbon fiber filled nylon. In some cases, foot 24 may be molded as two separate halves that can subsequently be affixed together. Needles 38 advance from the fixed needle guides 54, and are laterally directed into fittings 40 by receptacles 52, as illustrated in FIG. 6C. In general, a shape memory alloy such as Nitinol™ in its superelastic regime provides a particularly advantageous actuator wire for manipulating foot 24.

Referring now to FIG. 7, fittings 40 and suture 34 will be withdrawn proximally by the needles from needle receptacles 52. To releasably support fittings 40 and suture 34 and avoid entanglement of the suture in the needles, suture 34 is fittingly received within a slot 58 that extends laterally from needle receptacles 52. As the needles pull the fitting axially from needle receptacles 52, suture 34 is pulled from slot 58 and free from foot 24. Bending of the suture proximally within the suture slot can also locally increase the suture width, so that the interaction between the bent suture and the slot can help hold the fitting in the recess.

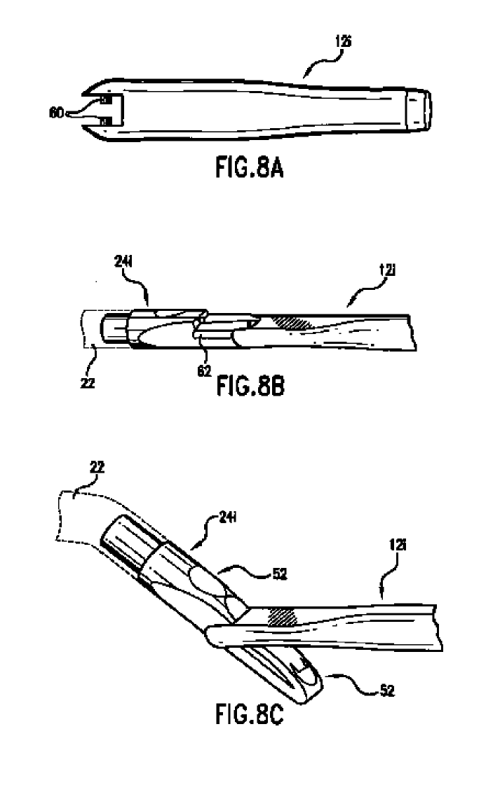

A wide variety of foot actuation mechanisms might be used within the scope of the present invention. A first alternative foot actuation arrangement is illustrated in FIGS. 8A-C. In this embodiment, a shaft 12i has pins 60 which ride in associated slots 62 of a foot 24i. Proximal motion of an actuation wire causes foot 24i to move axially and rotationally, with pins 60 sliding along slot 62, and the foot pivoting about the pins. In this embodiment, guidebody 22 extends directly from the foot, as illustrated in FIG. 8C.

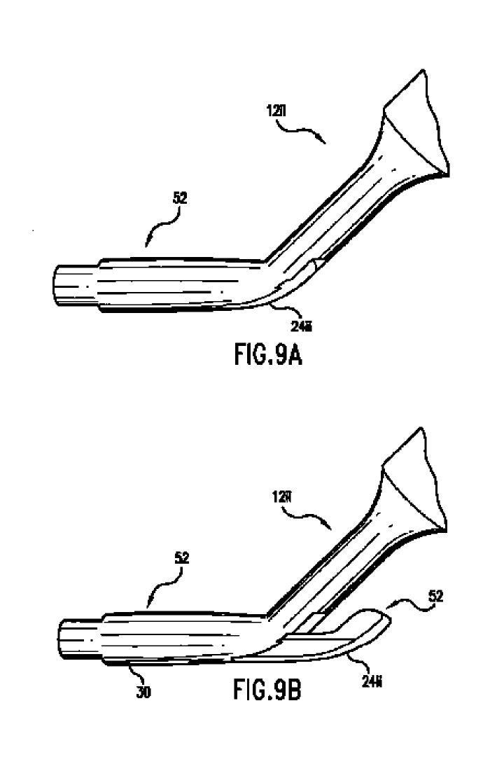

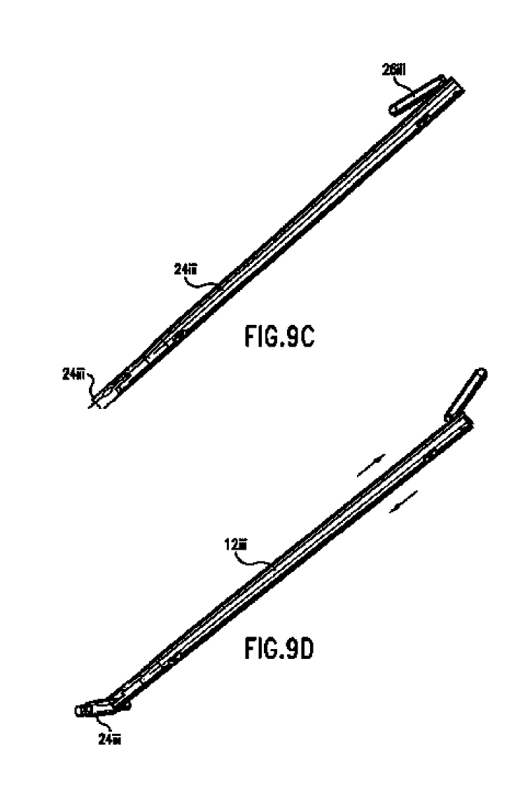

A still further alternative foot actuation mechanism is illustrated in FIGS. 9A and B. In this embodiment, slidable foot 24ii is slidingly received within a receptacle 30 of shaft 12ii. Sliding of the foot 24ii from the parked position of FIG. 9A to the deployed position of FIG. 9B places the needle receptacles 52 in the paths of needles from the shaft 12ii without pivoting of the foot. Guidebody 22 (see FIG. 1) will extend here from a distal end of shaft 12ii at a fixed angle from the shaft. Optionally, insertion through the tissue tract may be facilitated by including an additional bend in the shaft axis adjacent the guidebody on many embodiments.

Yet another foot actuation mechanism can be understood with reference to FIGS. 9C and D. Shaft 12iii is formed in two parts, which slide axially relative to each other when foot actuation lever 26iii moves, using an offset crank arrangement. A similar offset crank supports foot 24iii, so that the sliding shaft parts cause the foot to pivot as shown.

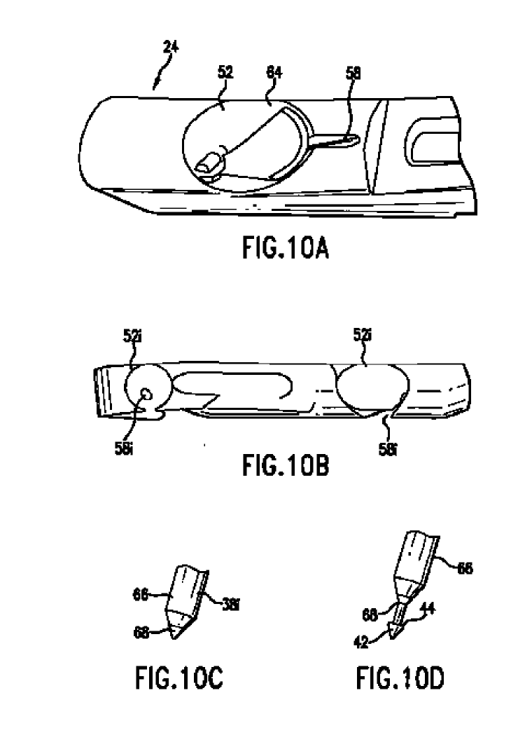

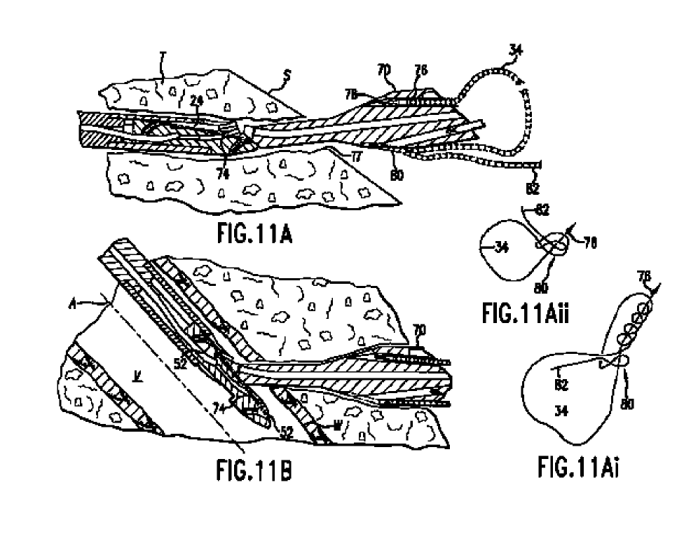

A variety of features may be included in the articulatable foot, the needle receptacle, and/or the needle to avoid tangling of the needle in the suture as the needle is directed to the fitting. As illustrated in FIG. 10A, a moveable flap 64 may extend over slot 58 so that the advancing needle slides along the flap toward the fitting, rather than entering the slot and engaging the suture directly. Flap 64 may be affixed along one side of the slot, with the other side of the flap flexing into the receptacle to release the suture from slot 58 when the fitting and suture are withdrawn by the needle.

An alternative mechanism for avoiding entanglement of the needle with the suture is illustrated in FIG. 10B. In this embodiment, needle receptacles 52i have tangential slots 58i which extends substantially tangentially to the surface of the receptacle. As a result of this tangential arrangement, a needle entering the receptacle 52i will be directed toward the fitting contained therein, but will generally not be able to enter and advance within the tangential slot 58i so as to become entangled with the suture. As illustrated in this embodiment, the slots may optionally extend laterally through the foot so that the loop of suture can be pulled from one side of the shaft without interference.

A still further alternative mechanism for avoiding entanglement between the suture and the needle is illustrated in FIGS. 10C and D. Two-part needle 38i includes an outer sheath 66 and an inner core 68. The parts of these needles initially advance together into the receptacles with the needle core 68 withdrawn so that the needle presents a smooth tapered tip (the combined tip preferably being larger in diameter than the slot containing the suture) as illustrated in FIG. 10C. Once two-part needle 38i is fully positioned within the needle receptacle, needle core 68 may extend axially to expose barbed tip 42 and recessed engagement surface 44 and to secure the needle to the fitting within the needle receptacle. In the exemplary embodiment of FIGS. 4 and 5, barbed tip 42 is formed integrally with the rest of the needle structure, but the tip has a larger cross-section than radial slot 58 containing the suture 34. As a result, the barbed tip is unable to enter the slot, thereby avoiding entanglement between the needle and suture.

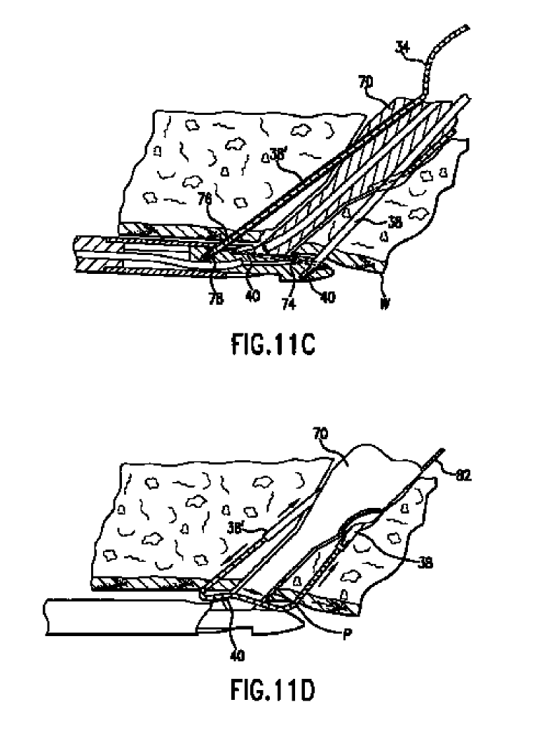

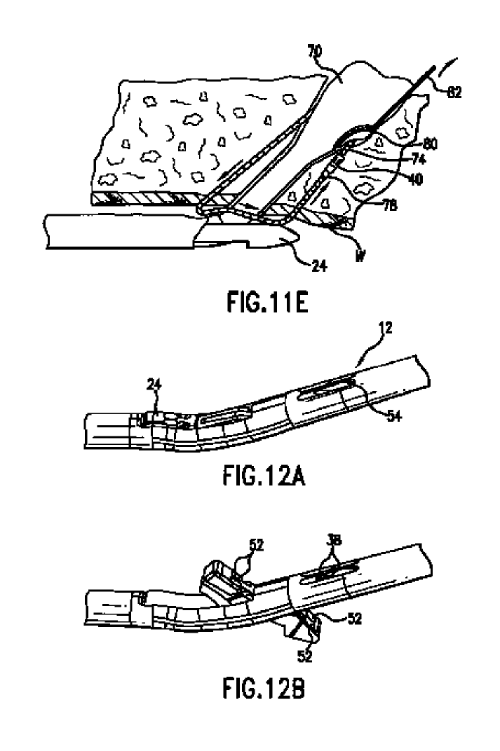

An alternative vessel closure probe 70 will be explained with reference to FIGS. 11A through 11E. This embodiment includes an articulatable foot 24 having a pair of needle receptacles 52, as described above. Although each needle receptacle 52 contains a fitting 40 for coupling a flexible filament to a tip of an associated needle, the filament in this case comprises a short length of suture 74 (or some temporary connecting filament, as shown schematically in phantom in FIG. 11A) spanning directly between the needle receptacles. Rather than pulling the two ends of an extended loop through the needle paths and proximally out the tissue tract for tying, closure system 70 advances a single end of the suture distally along one needle path, across the puncture, and then proximally along the other needle path. To provide this interaction, at least one needle includes means for attaching suture 34 to short suture 74, here in the form of a detachable coupling structure carried on the at least one needle. This structure facilitates the use of a pre-tied knot.

Referring now to FIGS. 11A and B, the distal end of probe 70 advances distally through skin S and into a tissue T of the patient while the probe is in the small profile configuration with foot 24 aligned along the axis of the probe. Here, however, an end 76 of suture 34 is affixed to a detachable needle tip 78 of a hollow needle 38′. Detachable tip 78 comprises a fitting having an opening receiving an end of suture similar to fitting 40, attached to a barbed needle end (similar to that of needle 38). Suture 34 may extend proximally within hollow needle 38 where the needle has an open channel along its length, may exit the hollow needle just proximally of detachable tip 78, or may be disposed alongside a solid needle. Needle 38 (opposite hollow needle 38′) has a fixed barbed tip, as described above, and a bight of suture 80 is releasably attached to the probe shaft encircling the opening of needle guide 54 of the fixed tip needle. The bight of suture may be releasably disposed within a slot of the probe, may be temporarily held in place by a weak adhesive or coating, or the like. A second end 82 of suture 34 extends proximally along the shaft of the probe, the second end of the suture optionally also being releasably held along the shaft.

Bight 80 will define a knot when first end suture passes therethrough, as can be understood with reference to FIGS. 11Ai and 11Aii. Bight 80 will often include more than one loop, and may be pre-arranged so as to define a square knot (using the layout schematically illustrated in FIG. 11Ai), a clinch knot (FIG. 11Aii), or a variety of known or new surgical knots.

Probe 70 advances along tissue tract TT to puncture P in blood vessel V. Once foot 24 is disposed within a blood vessel V, a pull wire moves the foot proximally and pivots the foot laterally so that the foot extends along an axis A of the vessel, as illustrated in FIG. 11B. The foot can then be pulled proximally against an inner surface of the vessel wall W to ensure that the needle receptacles 52 are properly positioned.

As can be understood with reference to FIGS. 11C and D, hollow needle 38′ and needle 38 advance to engage fittings 40 within receptacles 52. Hollow needle 38′ draws first end 76 of suture 34 distally through vessel wall W, and detachable tip 78 is secured into an associated fitting 40 using the barb and tab interaction described above. As short suture 74 extends between fittings 40, and as detachable tip 78 can pull free of hollow needle 38′ when the needles are withdrawn, this effectively couples needle 38 to first end 76 of suture 34. The detachable tip riding partially within the hollow needle (or vice versa) so that the assembly remains together under compression. Hence, needle 38 can pull the suture distally along the needle path formed by hollow needle 38′, across the puncture P, and proximally along the needle path formed by needle 38, as illustrated in FIG. 11D.

FIGS. 11D and E show that the knot can be completed by pulling needle 38, short suture 74, and second end 76 of suture 34 (together with the fittings 40 and detachable needle tip 78) proximally through bight 80. Second end 82 of suture 34 can be pulled to free bight 80, and the ends of the suture can be tightened and the probe removed to provide permanent hemostasis.

It will be recognized that removal of probe 70 can be facilitated by coupling first end 76 to bight 80 over an outer surface of the probe, and by arranging suture 34 and hollow needle 38′ so that the suture can pull free of the needle when detachable tip 78 is released, for example, by having the suture exit the needle proximally of the tip through a channel that extends to the tip so that the needle does not encircle the suture. By including such provisions, after foot 24 is returned to the narrow configuration, the probe can be pulled proximally from the tissue tract leaving the pre-tied knot in place.

Alternative arrangements (using the detachable needle ends of probe 70) are possible to provide the benefit of a pre-tied knot and the like for closure of a vessel puncture. For example, a probe having a pair of needles in which each needle included a detachable tip might be used to pull first end 76 through a bight, so that the bight need not encircle the needle path of one of the needles.

In some cases, particularly for closure of large punctures, it may be advantageous to provide multiple suture loops across the puncture, either in parallel, in an “X” pattern, or the like. As illustrated in FIGS. 12A and B, the present invention encompasses the use of more than two needles and associated receptacles, fittings, sutures, and the like. Multiple loop systems may have four, six, eight, or more needles, or may even have odd numbers of needles and fittings, particularly where one or more fittings have a plurality of suture ends extending therefrom. This allows a wide variety of stitching patterns to be provided by such multiple loop probes.

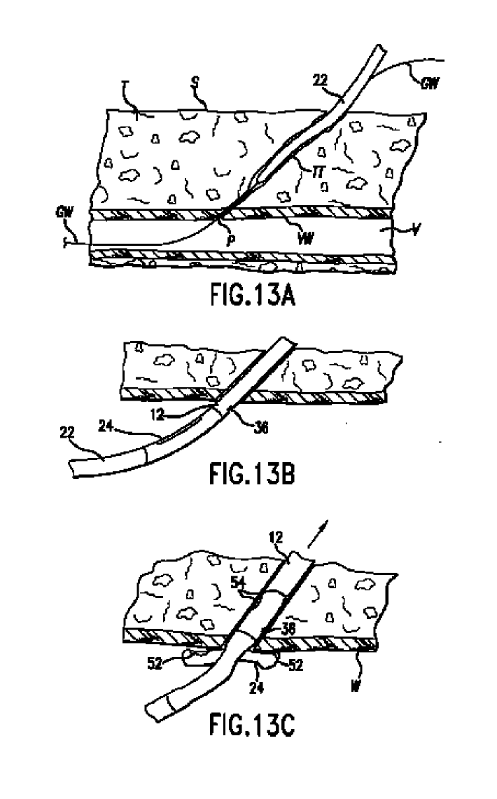

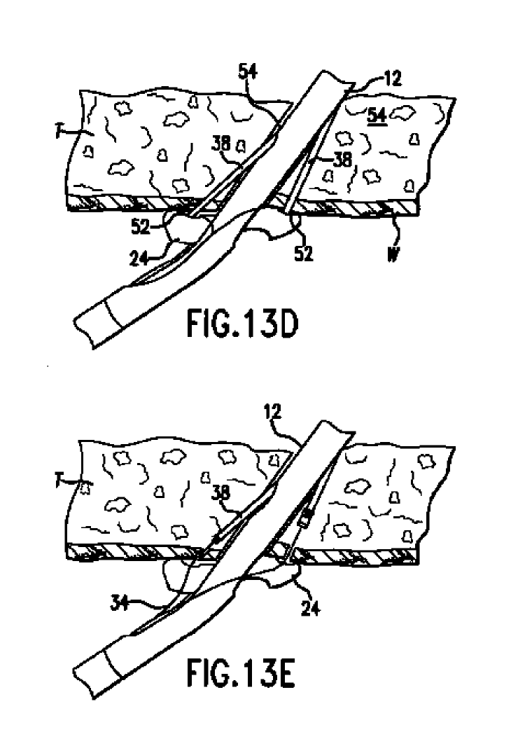

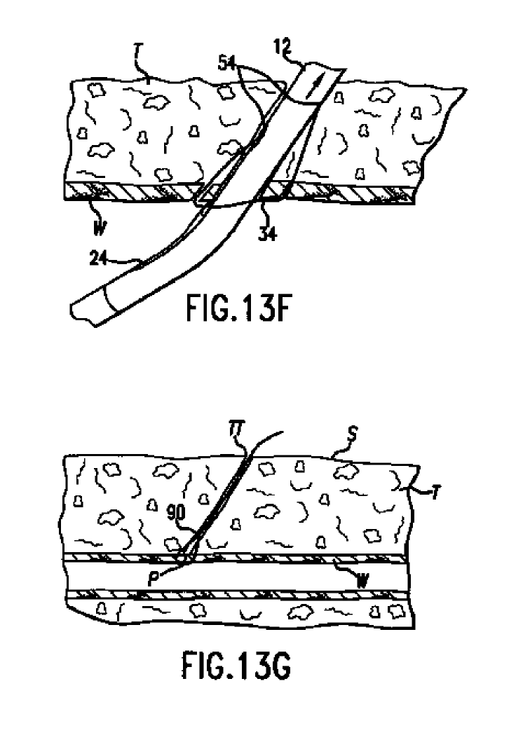

The method of use of the probes of FIGS. 1-7 can be understood with reference to FIGS. 13A-G. After accessing a blood vessel V (often using the Seldinger technique), a guidewire GW is left extending into skin S and down through tissue T along tissue tract TT. Guidewire GW enters vessel V through a puncture P in vessel wall W, and extends along the vessel throughout many endovascular procedures. As illustrated in FIG. 13A, distal guidebody 22 is advanced over the guidewire GW in a monorail fashion, so that the guidewire helps to direct the probe along the tissue tract TT and into the vessel through puncture P. FIG. 13B shows that when sensor 36 is disposed within the vessel, blood can flow from the sensor port and through a lumen in shaft 12 to the proximal handle to notify the operator that foot 24 has been advanced far enough for deployment.

Deployment of the foot is effected by actuation of the foot deployment handle, as described and illustrated above with reference to FIGS. 2 and 2B. As described above, guidebody 22 helps to align the probe with the axis of vessel V. Guidebody 22 may be set at an angle and/or offset relative to shaft 12 as appropriate to aid in alignment with a particular vessel access technique. As shown in FIG. 13C, the deployed foot 24 extends laterally from the shaft, so that foot 24 adjacent receptacles 52 can be drawn up against vessel wall W by gently pulling shaft 12. Hence, the foot helps to accurately position the needle guides 54 at a distance from the vessel wall.

Referring now to FIG. 13D, flexible needles 38 are deflected laterally by needle guides 54 toward receptacles 52 of the deployed foot. As a result, the needles advance in cantilever both distally and laterally when needle actuation handle 20 is pressed (see FIG. 2C), and the tapering surfaces of receptacles 52 help to push the needles back into alignment with the fittings so as to overcome any unintended deflection of the needles by tissue T or vessel wall W. This ensures that needles 38 securingly engage fittings 40 within receptacles 52, thereby coupling the ends of suture 34 to the needles. While suture 34 is here illustrated running along the side of shaft 12 outside foot receptacle 30 to a lumen within guidebody 22, it should be understood that the suture loop might instead extend proximally in a lumen of shaft 12, might be routed through the foot and/or foot receptacle, and/or might be stored in a spool adjacent foot 24. Regardless, suture 34 should able to pull free of the probe between its ends to form a continuous loop across puncture P.

Referring now to FIGS. 13E and F, fittings 40 and the ends of suture 34 are drawn proximally through the vessel wall W along the needle paths formed by needles 38. Optionally, the needles may be withdrawn proximally out of the tissue tract and clear of shaft 12, or they may remain coupled to the shaft within needle guides 54. The foot actuator is moved to store foot 24 along shaft 12, and the shaft can then be pulled proximally from the tissue tract. Guidebody 22, which may comprise a soft, compliant polymer, may temporarily extend at least partially into tissue tract TT and through puncture P to help reduce the loss of blood until the loop is secured.

Now referring to FIG. 13G, once shaft 12 has been withdrawn sufficiently to expose needle guides 54, the ends of the suture loop can be grasped by the operator. Tying of a knot in suture 34 can then proceed in a conventional manner. The use of a clinch knot may facilitate gradual tightening of the knot while removing guidebody 22, although a wide variety of knot and knot advancing techniques might be used.

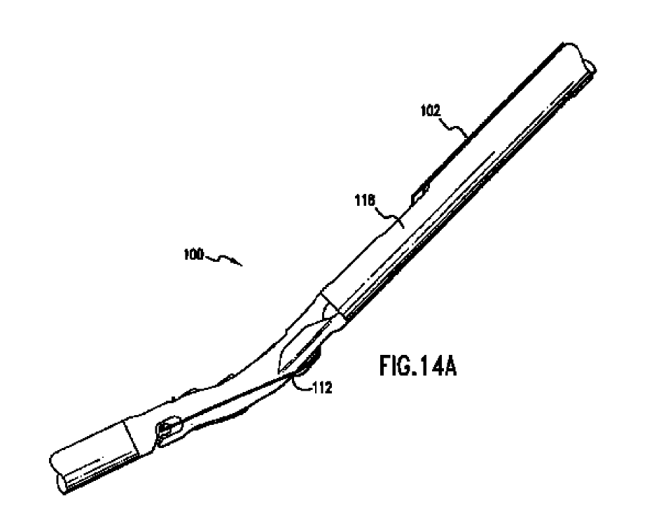

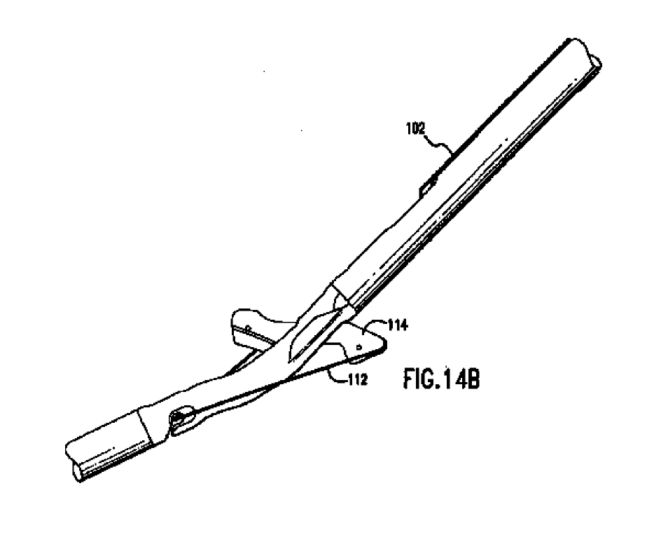

FIGS. 14A and 14B show an embodiment of a vessel closure device 100. This embodiment includes an articulatable foot 114 (FIG. 14B) having a pair of penetrator receptacles (described below). Although each penetrator receptacle contains a fitting (or cuff) for coupling a flexible filament to a tip of an associated penetrator, the filament in this case may be a short length of suture such as a link 112 spanning directly between the penetrator receptacles. Rather than pulling the two ends of an extended loop through the needle paths and proximally out the tissue tract for tying, closure system 100 advances a single end of the suture distally along one needle path, across the puncture, and then proximally along the other needle path. To provide this interaction, at least one needle includes means for attaching suture 102 to the link 112, here in the form of a detachable coupling structure carried on the at least one needle. This structure facilitates the use of a pre-tied knot.

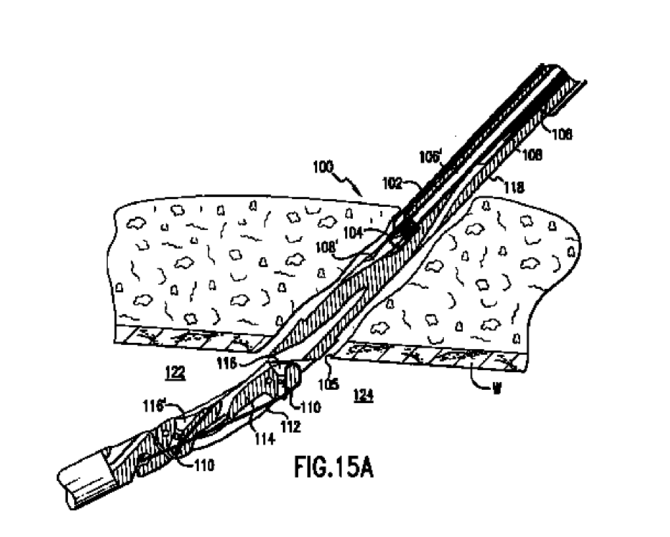

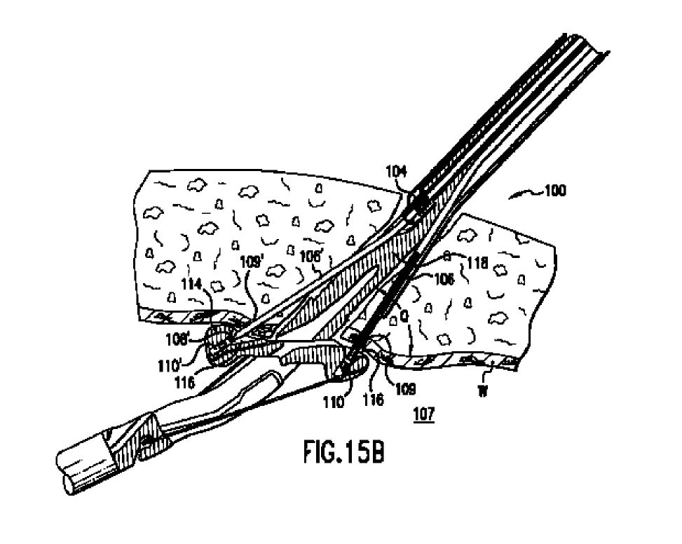

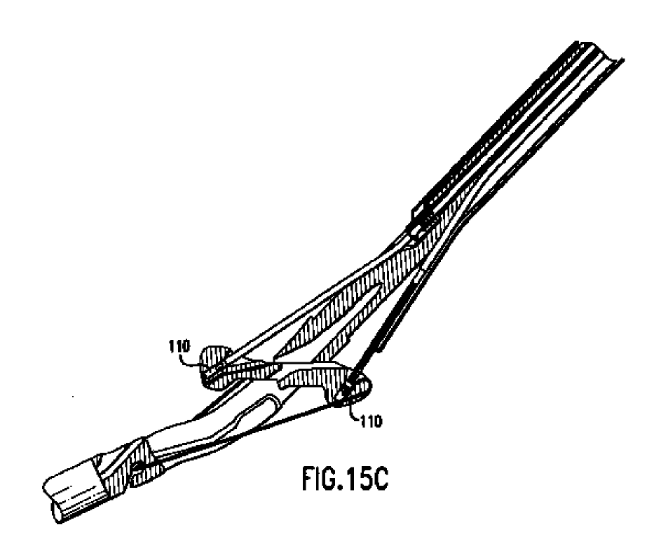

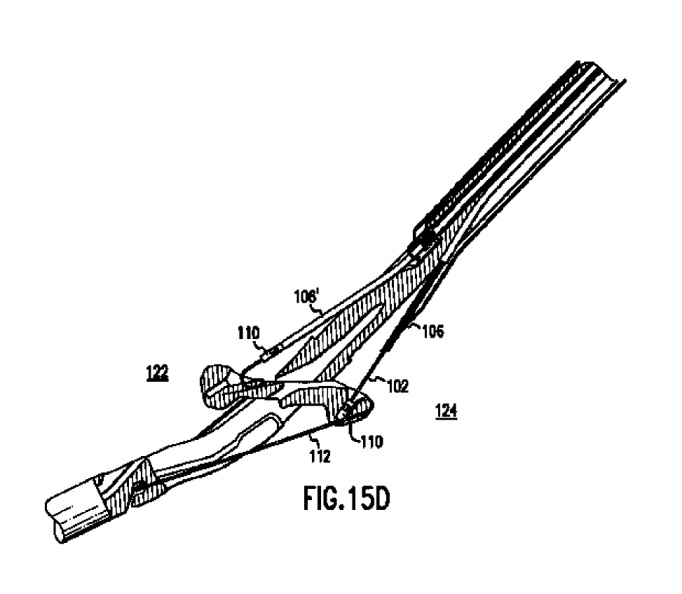

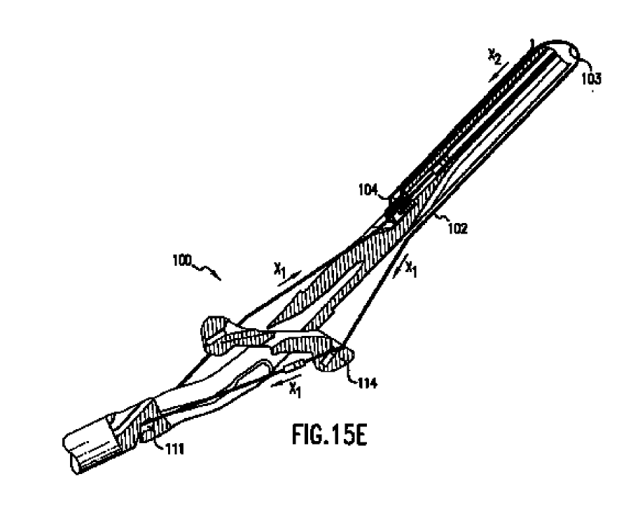

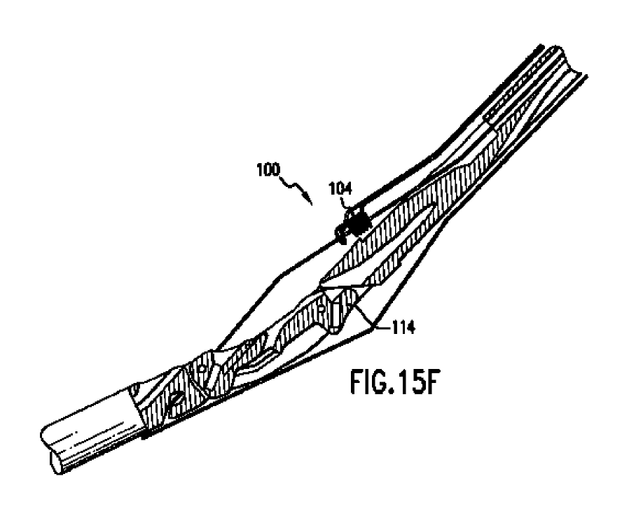

FIG. 15A shows a side, cross-sectional view of the device 100 in a position prior to deployment of the foot 114. The device 100 has been advanced through the incision 105 in the arterial wall W. For ease of description, reference numeral 122 indicates the anterior side of the device, and reference numeral 124 denotes the posterior side of the device. Device 100 has a rigid shaft 118 that has channels defined therein to carry the elongate bodies or penetrators 106 and 106′. Penetrator 106′ may also be referred to as the anterior penetrator, and penetrator 106 may be referred to as the posterior penetrator. For purposed of description and not limitation, the anterior penetrator 106′ carries the pre-tied knot 104, and posterior penetrator 106 carries the detachable coupling structure or penetrator tip 108. Anterior penetrator 106′ defines a penetrator tip 108′ at its distal end.

The articulatable foot 114 includes anterior and posterior penetrator receptacles 116′ and 116, respectively. These receptacles are also referred to as cuff pockets. Cuffs 110 are shown positioned in cuff pockets 116′ and 116. A link 112 extends between the cuffs 110.

FIG. 15B shows the foot 114 deployed so as to position the cuff pockets 116 to receive the first and second penetrators 106′ and 106. As shown in FIG. 15B, the anterior penetrator 106′ has the pre-tied knot 104 disposed about a proximal portion of its length. Alternatively, the pre-tied knot 104 may be disposed about the periphery of a knot tube, through which the anterior penetrator 106′ may pass (as described in further detail below).

FIG. 15B illustrates the suturing device 100 deployed within a lumen 107 in accordance with an embodiment of the present invention. As may be seen with reference to the Figure, the suturing device 100 includes an elongate body 106′ having a penetrator tip 108′. The elongate bodies 106 and 106′ deploy to form penetrations 109 and 109′ within the vessel wall W. The configuration of the penetrator tip 308 allows penetration of the vessel wall W immediately surrounding the incision 105 to form the penetration 309. As such, the penetration of the penetrator tip 108 through the tissue wall W allows for passage of the elongate body 106 through the tissue and into the lumen 107. The elongate body 106 holds the suture 102 as the elongate body 106 passes through the tissue wall W immediately adjacent the incision 105 and into the foot 114.

As may be seen with reference to FIG. 15B, in this embodiment, the foot 114 has a single unit design where the cuffs 110 and 110′ are disposed on opposite sides of the suturing device 100 and the foot 114. This orientation allows balance of forces during the deployment of the elongate bodies 106 and 106′, thereby allowing precise suturing and minimizing the possibility of incorrectly suturing the incision 105. Also, as may be seen with reference to the Figure, the suturing device 100 delivers the suture longitudinally relative to the lumen 107, thereby minimizing arterial diameter constriction. Likewise, in this embodiment, the foot 114 is positioned at an angle “Q” relative to the shaft 118 of the suturing device 100. Preferably, the angle “Q” is in a range between about 20 degrees and about 60 degrees and more preferably is about 40 degrees. The angle “Q” approximates the puncture angle commonly used to access the femoral artery. The angle Q and the rigid character of the shaft 118 serve to provide accurate, virtually simultaneous “cuff capture” by both the anterior and posterior penetrators. Moreover, since the device 100 is preferably used without an introducer sheath, the rigid nature of the shaft 118 provides the control of the travel of penetrators as they move distally to engage the cuffs. The device 100 can therefore be used in the same femoral artery access puncture without disturbing the existing tissue tract and causing undue discomfort to the patient.

When both the elongate bodies 106 and 106′ and the suture 102 pass through the lumen wall W and into the lumen 107, the elongate bodies 106 and 106′ engage with the foot 114. The penetrator tip 108 and anterior penetrator tip 108′ of the elongate bodies 106 and 106′ engage with cuffs 110 and 110′ of the foot 114. The cuffs 110 and 110′ include a link 112 that connects the cuffs 110 and 110′ to one another. It should be noted that the cuffs 110 and 110′ facilitate connection of the penetrator tip 108 with the anterior penetrator tip 108′ such that the penetrator tip 108 and the anterior penetrator tip 108′ are coupled to one another via the link 112.

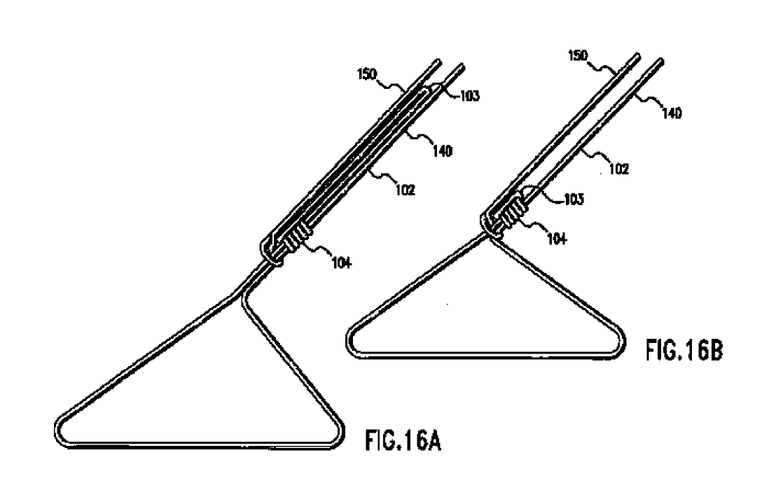

FIGS. 16A and 16B show the suture bight in the pre-deployed state (FIG. 16A) and the deployed state (FIG. 16B). The suture 102 is arranged to provide the pre-tied knot 104 that automatically travels down from the shaft of the device where it is stored prior to delivery to the tissue wall. The loop 104 of suture 102 serves to pull the knot 104 down the rail portion 140 of the suture during deployment. It should be noted that it would be desirable to be able to distinguish the ends 140 and 150 of the suture 102 during deployment so that the correct end is pulled by the operator to advance the knot. Should the non-rail end be pulled, the knot may be prematurely tightened before it is advance to its deployed position at the wall of the vessel.

The ends of the suture may be distinguished from each other by changing the color of one end (e.g. with dye), providing an attachment on one end (e.g. shrink wrap tubing, a bead, etc.) or with the suture itself (e.g. tying a knot in one end).

FIG. 15C shows the penetrator tips fully deployed into and engaged with the cuffs 110. FIG. 15D shows the penetrators being retracted after the tips have engaged the cuffs 110. On the anterior side 122, the penetrator 106′ is pulling the anterior cuff 110 distally. On the posterior side 124, the penetrator tip 108 has been disengaged from the penetrator 106, via a mechanism described below. As shown in FIG. 15D, the link 112 is now coupled to one end of the suture via posterior cuff 110. Suture 102 is also shown exiting the posterior penetrator shank via an opening in the side of the penetrator shank.

Referring to FIG. 15E, after deployment of the foot 114, the suture 102 moves as indicated by directional arrows X1. As the suture 102 moves, a suture loop 103 also moves in a direction indicated by directional arrow X2 towards the foot 114 and the incision (not shown). The suture 102 moves through the foot 114 and through an opening distal to the foot 114 that defines a suture-bearing surface 111. The suture-bearing surface 111 is disposed at a distal end of the suturing device 100 separate from the foot 114, in this embodiment. The suture bearing surface 111 bears forces placed on the suture 102 during suturing. As such, the suture-bearing surface 111 minimizes forces placed on an incision during incision tensioning, thereby minimizing the possibility of damaging tissue immediately surrounding the incision. In this embodiment, the suture bearing 111 is a slot disposed at a distal end of the suturing device 100, which includes a passage for the suture 102 during incision suturing as shown with reference to the Figure.

As the suture loop 103 and the suture 102 move, the pre-tied suture knot 104 also moves in the same direction as the suture loop 103 towards the foot 114 and the incision. The suture loop 103 continues to move the pre-tied suture knot 104 towards the incision until the suture 102 and the pre-tied suture knot 104 suture the incision formed in the arterial wall. It should be noted that a suture trimmer might be used to assist the delivery of the knot 104 to an arteriotomy. The suture trimmer may be any device suitable for pushing the knot towards the arteriotomy and trimming suture immediately adjacent the knot 104 once the knot is tightened.

Now making reference to FIG. 15F, the suturing device 100 delivers the pre-tied suture knot 104 to the incision and the foot 114 is returned to its non-deployed position. The penetrators (not shown) have been retracted, the link has been fully retracted through the knot, and the knot has been advanced to the vicinity of the arterial wall. When the body of the device is removed, a stitch will remain in place across the incision in the artery. It should be noted that embodiments of the device described herein place a stitch of suture in a longitudinal orientation with respect to the vessel so as to minimize transverse vessel constriction and also to take advantage of the transverse orientation of the fibers of the vessel tissue.

FIGS. 16A and 16B show the suture bight in the pre-deployed state (FIG. 16A) and the deployed state (FIG. 16B). The suture 102 is arranged to provide the pre-tied knot 104 that automatically travels down from the shaft of the device where it is stored prior to delivery to the tissue wall. The loop 104 of suture 102 serves to pull the knot 104 down the rail portion 140 of the suture during deployment. It should be noted that it would be desirable to distinguish the ends 140 and 150 of the suture 102 during deployment so that the correct end is pulled by the operator to advance the knot. Should the non-rail end be pulled, the knot may be prematurely tightened before it is advanced to its deployed position at the wall of the vessel.

The ends may be distinguished from each other by changing the color of one end (e.g. with dye), providing an attachment on one end (e.g. shrink wrap tubing, a bead, etc.) or with the suture itself (e.g. tying a knot in on end).

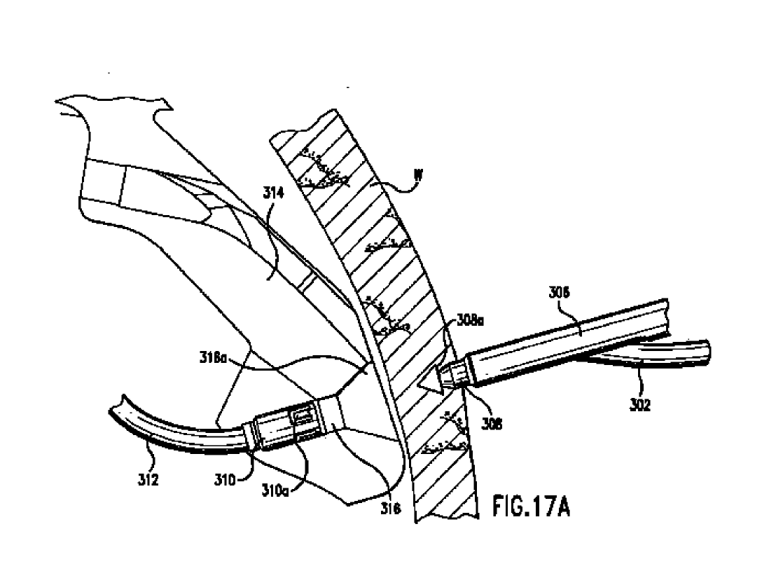

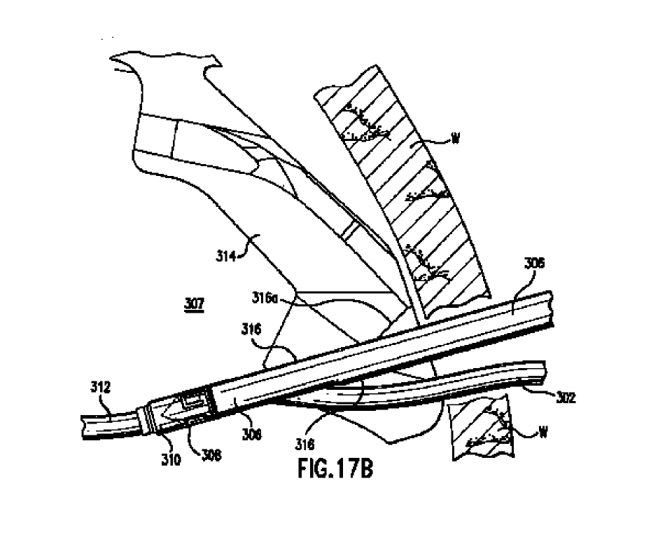

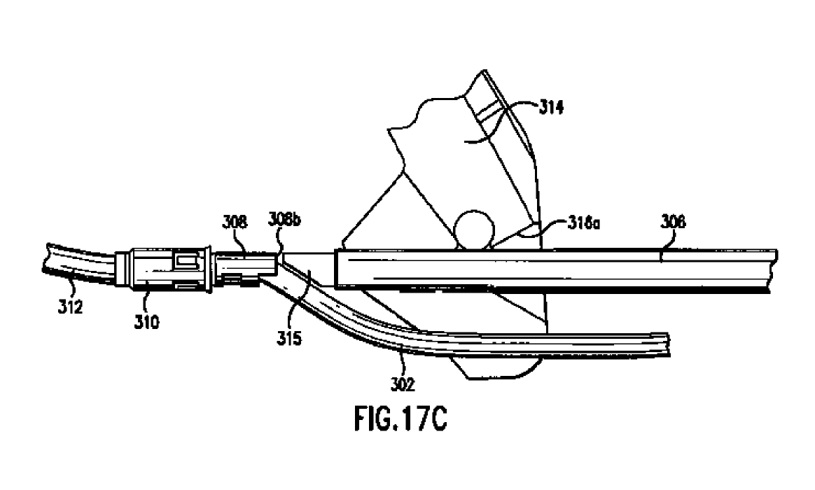

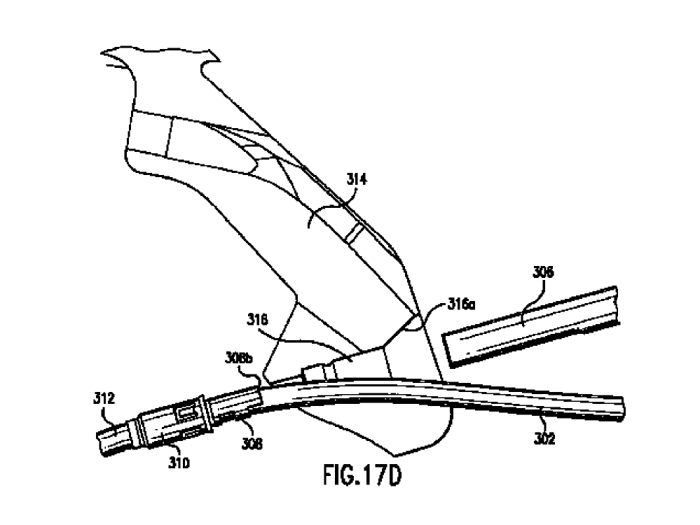

FIG. 17A shows an enlarged detail of the posterior portion of the foot of one embodiment of suturing device 300. In an accordance with an embodiment of the present invention, the elongate body 306 may be any type of structure capable of penetrating the wall of a lumen, such as an artery, a blood vessel, or the like. In addition to the penetration capability, the elongate body 306 may be a hollow tube capable of holding suture. Examples of such structures may include a hypodermic needle or the like. The suturing device 300 stores the elongate body 306 within its shaft (not shown). As previously described with reference to FIGS. 2A through 2C, a user deploys a handle (not shown) of the suturing device 300 thereby deploying the elongate body 306 and the penetrator tip 308. During deployment, the elongate body 306 and the penetrator tip 308 penetrate the lumen wall W immediately surrounding the incision 305 and enter the lumen 307 of a patient, as shown with reference the following FIG. 17B.

Once the penetrator tip 308 engages with the cuff 310, the elongate body 306 and the penetrator tip 308, along with the cuff 310, proceed through the foot 314 and into the lumen 307. As may be seen with reference to FIG. 17B, the cuff 310 is pushed through the foot 314, such that the cuff 310 is pushed out of a pocket 316 and through the foot 314 into the lumen 307. Once the cuff 310 and the elongate body 306 enter the lumen 307, the penetrator tip 308 detaches from the elongate body 306 via a push mandrel 315 as shown with reference to FIG. 17C.

FIG. 17C illustrates the detachment of the penetrator tip 308 from the elongate body 306 in accordance with one embodiment of the present invention. Upon engagement of the penetrator tip 308 with the cuff 310, the push mandrel 315 is further advanced such that it contacts a proximal surface 308b of the penetrator tip 308, and further still until the penetrator tip 308 detaches from the elongate body 306. Upon detachment of the penetrator tip 308 from the elongate body 306, the push mandrel 315 and the elongate body 306 retract from the foot 314, as shown with reference to FIG. 17D.

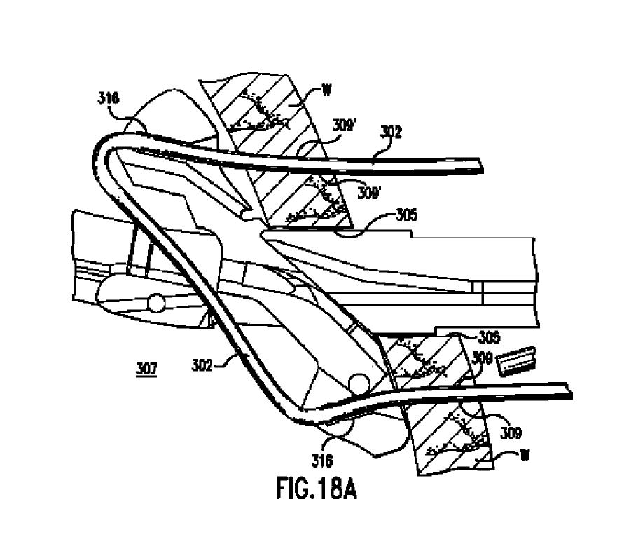

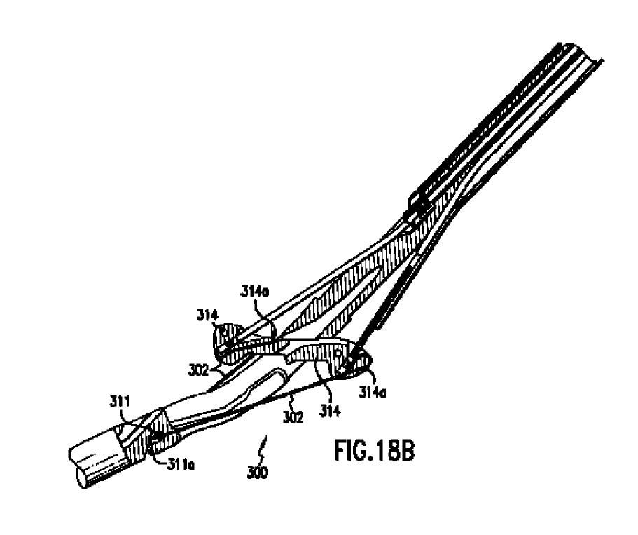

As shown in FIG. 17D, after the penetrator tip 308 detaches from the elongate body 306, the elongate body 306 retracts from the penetrator tip 308 and cuff 310. Meanwhile, on the anterior side of the device (not shown in FIG. 17D), the elongate body 306′ also includes the needle tip 308′ which engages with the cuff 310′ as previously described with reference to FIG. 15C. The needle tip 308′ does not disengage from the elongate body 306′ upon engagement with the cuff 310′. Therefore, during retraction of the elongate body 306′ from within the lumen 307, the needle tip 308′ also retracts from the lumen 307 through the penetration 309′. As the needle tip 308′ retracts through the penetration 309′, the elongate body 306′ also retracts the cuff 310′. As previously described, the cuff 310′ couples with the cuff 310 via the link 312. During retraction of the cuff 310′ through the penetration 309′, the cuff 310 and the suture 302 also retract through the penetration 309′, thereby drawing the suture 302 through the penetration 309′. It should be noted that the foot 314 may provide suture bearing surface for the suture 302 during operation of the suturing device 300, as shown with reference to FIG. 18A.

FIG. 18A shows an embodiment of the present invention illustrating the passage of the suture 302 through the lumen 307 and the passageways 309 and 309′. As may be seen with reference to the Figure, the cuff pockets 316 of the foot 314 provide a suture-bearing surface for the suture 302 as the suture 302 is drawn through the passageways. The suture bearing surfaces of the foot 314 minimize the possibility of the suture 302 damaging tissue surrounding the incision 305.

In another embodiment shown in FIG. 18B, the suturing device 300 also provides a suture bearing surface for the suture 302. During retraction of the elongate bodies 306 and 306′ from the lumen 307, the suture 302 retracts through the foot suture bearing surfaces 314a and the suture-bearing surface 311 formed distally of the foot. The distal suture bearing surface 311 and the foot suture bearing surfaces 314a guide the suture 302 in order to minimize the possibility of the suture 302 damaging the patient during retraction of the elongate bodies 306 and 306′ from the lumen 307. In this embodiment, suture-bearing surface 311 is a slot defined in the body of the device distal of the foot. The slot includes a passage for the link and suture, and an edge 311a. It is contemplated that the edge 311a may contact the edge of the incision in the artery and become caught on the adventitia of the blood vessel. Various devices may be provided, such as flaps, o-rings, etc., that provide a smoother transition over the slot and edge 311a as the device is inserted through the incision.

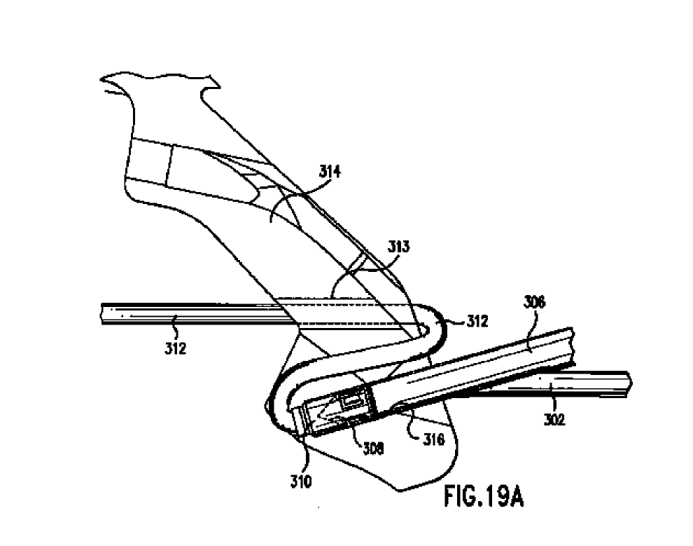

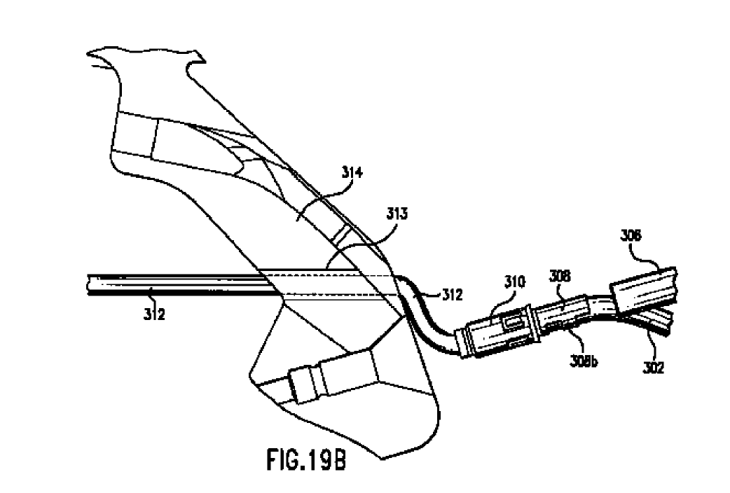

FIGS. 19A and 19B illustrate an alternative embodiment of the present invention for releasing the cuff 310 from the foot 314. In this embodiment, the foot 314 includes link passageway 313 through which the link 312 passes. After the elongate body 306 engages the penetrator tip 308 with the cuff 310, the elongate body 306, during refraction from the foot 314, removes the cuff 310 and the penetrator tip 308 from the foot 314. The force holding the penetrator tip 308 on the elongate body 306 overcomes the force holding the cuff 310 in the cuff pocket 316. Once the cuff 310 clears the foot 314 and attains the orientation shown with reference to FIG. 19B, the previously described push mandrel (not shown) detaches the penetrator tip 308 from the elongate body 306. Upon detachment of the penetrator tip 308 from the elongate body 306, the link 312, along with the cuff 310 and the penetrator tip 308, retracts through the passageway 313 via the link 312 and the elongate body 306′. In an alternate embodiment, the cuff 310 and penetrator tip 308 may be pulled off the elongated body 306 by tension in the link 312.

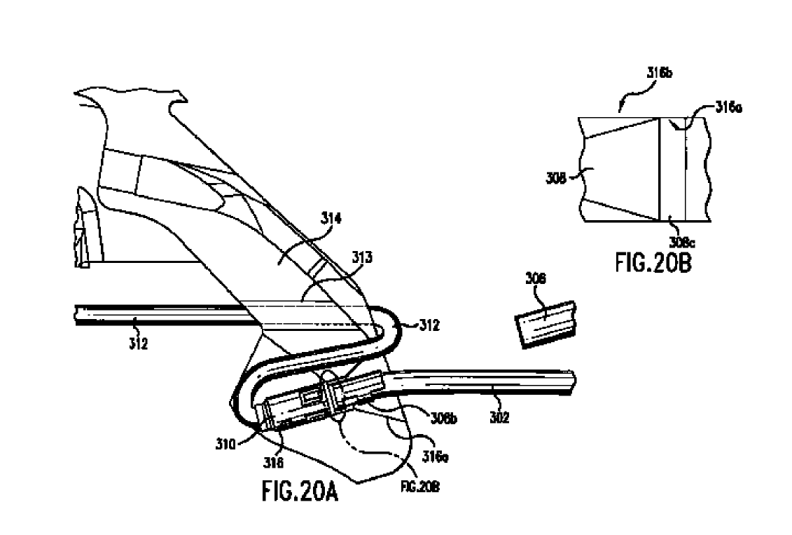

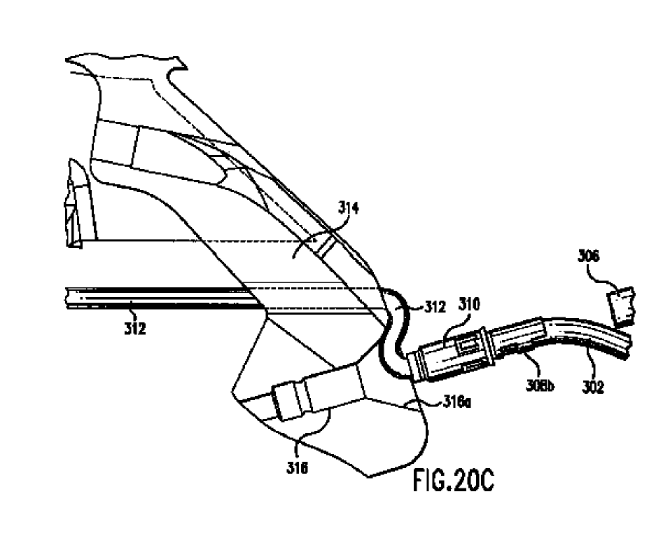

In yet another alternate embodiment shown in FIGS. 20A through 20C, the cuff 310 and penetrator tip 308 may be detached from the elongate body 306 before being removed from the cuff pocket 316. In this embodiment, after the elongate body 306 and the penetrator tip 308 engage with the cuff 310, the push mandrel 315 detaches the penetrator tip 308 from the elongate body 306, leaving it in the cuff pocket 316 to be removed by tension in the link 312, as shown in FIG. 20C.

It should be noted that other methods might be used to detach the penetrator tip 308 from the elongate body 306. These methods include, but are not limited to, detachment through friction or tension. Making reference to FIG. 20B, in an embodiment where friction between the cuff pocket 316 and the cuff causes detachment of the penetrator tip 308 from the elongate body 306, a surface 308c of the penetrator tip 308 frictionally engages with a cuff surface 316a of the cuff pocket 316. During retraction of the elongate body 306 from the foot 314, the frictional engagement between the cuff surface 316a and the penetrator tip surface 308c causes detachment of the penetrator tip 308 from the elongate body 306. In an embodiment where link tension causes detachment of the penetrator tip 308 from the elongate body 306, the link 312 is tensioned such that the link 312 is taut between the cuffs 310 and 310′. As such, the tension of the link 312 prevents movement of the cuff 310 out of the foot 314 along with the elongate body 306 during retraction of the elongate body 306 from the foot 314, thereby causing detachment of the penetrator tip 308 from the cuff 310.

After detachment, during retraction of the elongate body 306 and the elongate body 306′ (not shown), the link 312 may draw the cuff 310 and the penetrator tip 308 from the cuff pocket 316. As discussed earlier, the cuff 310′ engages with the elongate body 306′ and pulls the cuff 310 via the link 312 as the elongate body 306′ retracts from the lumen 307. As such, retracting the link 312 pulls on the cuff 310, thereby pulling the cuff 310 from the cuff pocket 316 and through the lumen 307 along with the suture 302, as shown with respect to FIG. 20C.

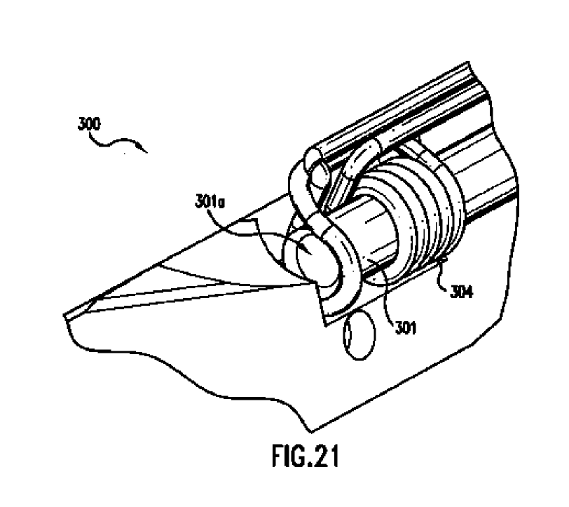

FIG. 21 shows the pre-tied suture knot 304 disposed about a periphery of a knot tube 301. In this embodiment, the knot tube 301 includes a hollow center 301a configured to allow passage of an elongate body (not shown) as the suturing device 300 sutures the incision. However, it should be noted that in an alternative embodiment of the present invention, the elongate body (not shown) might also store the suture 302. In the alternative embodiment, the suture 302 and the pre-tied suture knot 304 are disposed about a periphery of the elongate body where the pre-tied suture knot 304 may reside within a pocket (not shown) of the elongate body.

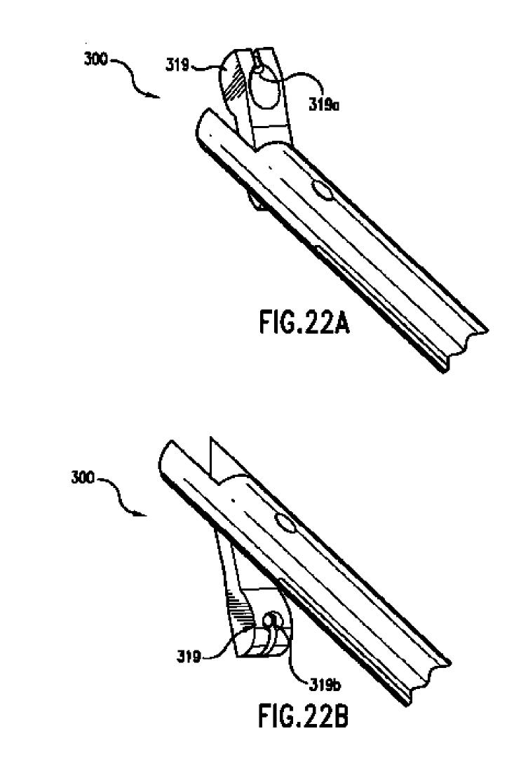

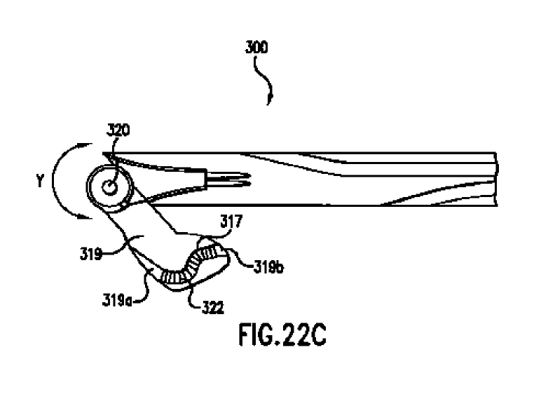

Embodiments of the suturing device of the invention may also include additional configurations for a foot, as shown with reference to FIGS. 22A through 22C. In this embodiment, the suturing device 300 includes a foot 319 having cuff pockets 319a and 319b. The configuration of the cuff pockets 319a and 319b allow the foot 319 to hold the cuffs 310 and 310′ during use of the suturing device 300. The foot pivots from a first orientation shown with reference to FIG. 22A to a second orientation shown with reference to FIG. 22B via a hinge 320 as shown in FIG. 22C.

FIG. 22C shows the hinge 320, which allows rotation of the foot 319 in a direction indicated by directional arrow Y. The hinge 320 may be any device capable of rotatably coupling the foot 319 to the suturing device 300, such as pin assembly or the like. In addition to the hinge 320, the foot 319 includes a connector 322 that couples the cuffs 310 and 310′ with one another. The connector 322 also includes a flexible portion 322c (shown with respect to FIG. 22C) that allows flexing of the connector 322 as the connector 322 resides within passage 317 of the foot 314. The connector also includes ends 322a and 322b that facilitate connection with the penetrator tip 308 and the needle tip 308′ of the elongate bodies 306′ and 306.

In an embodiment of the present invention where the suturing device 300 employs the foot 319, during use of the suturing device 300, upon insertion of the suturing device 300 within the lumen 307, a user deploys the foot 319 as shown with reference to FIG. 22A. Upon deployment of the foot 319, the user deploys the elongate body 306 (not shown) that engages with the cuff 310 (not shown) as previously described. Once the penetrator tip 308 detaches from the elongate body 306 via the push mandrel 315, or other means previously described, the user rotates the foot 319 into the orientation shown with reference to FIG. 22B. Upon orientation of the foot 319 as shown with respect to FIG. 22B, the user deploys the elongate body 306′ (not shown) which engages with the cuff 310′ (not shown). After the elongate body 306′ engages with the cuff 310′, the user retracts the elongate body 306′ along with the cuffs 310 and 310′ and the suture 302 to suture an incision as previously described.

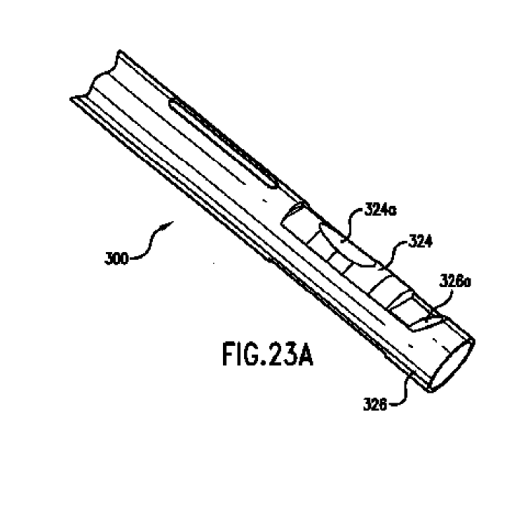

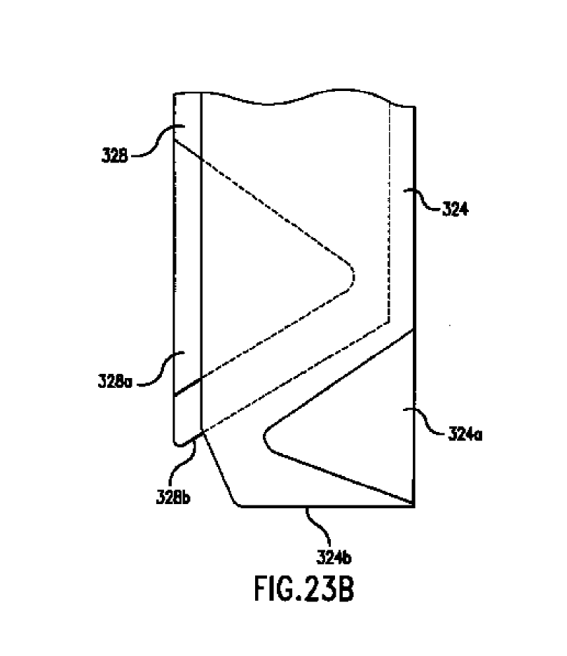

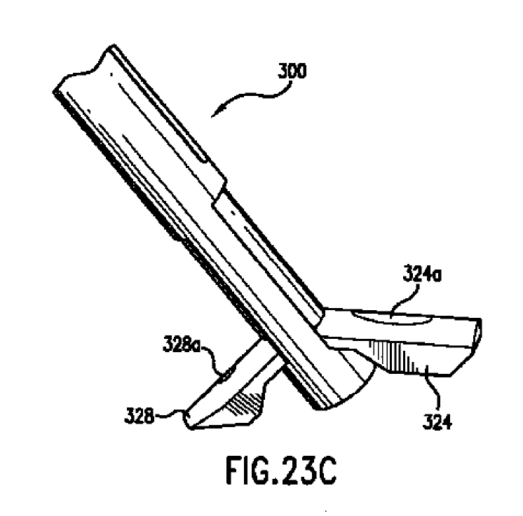

Another embodiment of the suturing device 300 includes feet 324 and 328 as shown with reference to FIG. 23A. FIG. 23A illustrates an embodiment of the present invention in which the suturing device 300 includes the feet 324 and 328. As may be seen with reference to FIG. 23B, the foot 324 is hollow such that the foot 328 fits within the foot 324 during both insertion and retraction of the suturing device 300 within the lumen 307. The feet 324 and 328 also include cuff pockets 324a and 328a and cam surfaces 324b and 328b. The configuration of the cuff pockets 324a and 328a allow placement of the cuffs 310 and 310′ within the feet 324 and 328 during use of the suturing device 300; allowing engagement of the elongate bodies 306 and 306′ during suturing. The cam surfaces 324a and 328a contact cam surfaces 326a in order to deploy the feet 324 and 328. Once the feet 324 and 328 deploy, the suturing device 300 attains the configuration shown with reference to FIG. 23C.

During use of a suturing device implementing the feet 324 and 328, a user inserts the suturing device into an incision as the foot 328 resides within the foot 324. Upon insertion of the suturing device within the incision, the user deploys the feet 324 and 328 by moving the feet 324 and 328 towards the cam surfaces 326a, in order to deploy the feet 324 and 328, as previously described. After deployment of the feet 324 and 328 within a lumen, the user deploys the elongate bodies 306 and 306′ whereby the penetrator tip 308 and needle tip 308′ engage with the cuffs 310 and 310′ residing within the cuff pockets 324a and 328a. Upon engagement with the cuffs 310 and 310′ the user retracts the elongate bodies 306 and 306′ and sutures the incision.

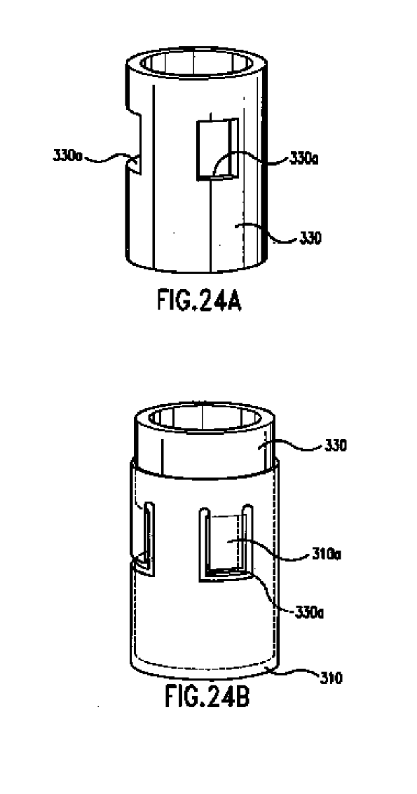

In addition to the alternative configurations for the foot of the suturing device 300, the suturing device 300 may also include alternative cuff configurations that allow engagement of the elongate bodies 306 and 306′ with the link 312. An example of such an alternative configuration is shown with respect to FIG. 24A. FIG. 24A illustrates a perspective view of an alternative embodiment of the penetrator tip 330. In this embodiment, a penetrator tip 330 includes mating surfaces 330a which engage with the previously described cuff tabs 310a of the cuff 310 when the penetrator tip 330 engages with the cuff 310, as shown with reference to FIG. 24B. As such, a user detaches the elongate body 306 from the penetrator tip 330 with the push mandrel 315 after engagement of the penetrator tip windows 330a with the cuff tabs 310, as discussed with reference to the penetrator tip 308 and the cuff 310. The mating surfaces 330a may be cut-outs, such as windows, formed within the penetrator tip 330. The elongate bodies 306 and 306′ may also engage with the link 312.

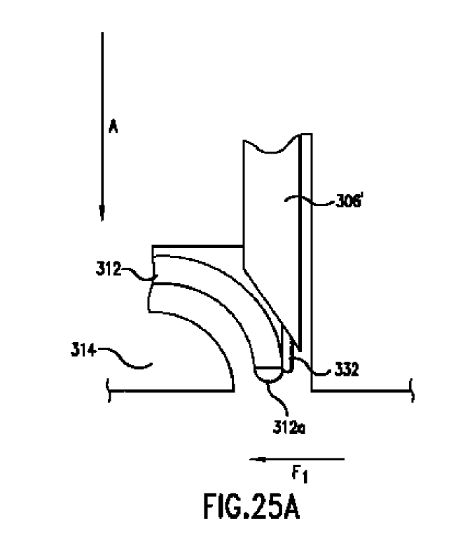

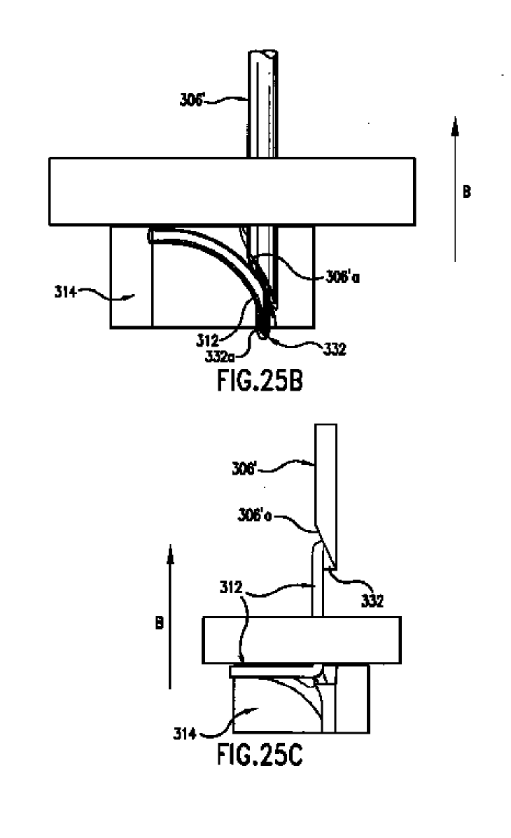

FIG. 25A shows an alternative method of coupling the elongate bodies 306 and 306′ with the link 312. In this embodiment, the elongate body 306′ includes a loop 332 (shown in FIG. 25B) which engages with the link 312 as the elongate body 306′ enters the foot 314. In this embodiment, the link 312 is constructed of a resilient material capable of flexing in response to the loop 332 contacting the link 312, such as polypropylene or any other material having spring-like characteristics. The elongate body 306′ moves in a downward direction as indicated by directional arrow A until the loop 332 comes into contact with an end 312a of the link 312. When the loop 332 contacts the end 312a, the loop 332 moves the end 312a in a direction F1 indicated by directional arrow F1. The catch 332 continues to move the end 312a of the link 312 in the direction F1 until the loop 332 contacts the end 312a, as shown with reference to FIG. 25B.

Referring to FIGS. 25A-C, the link 312 is constructed of a material having spring like properties. Therefore, when the loop 332a comes into contact with the end 312a, the resilient properties of the link 312 move the end 312a in a direction F2, as indicated by directional arrow F2 in FIG. 25A. The end 312a moves in the direction F2 such that the end 312a moves into the loop 332a, as shown with reference to FIG. 25B. Once the end 312a moves into the loop 332a, a user retracts the loop 332 along with the end 312a and the link 312 in a direction B as indicated by directional arrow B of FIG. 25C. As the loop 332a and the catch 332 move in the direction B, the loop 332a clamps the link 312 against a surface 306′a of the elongate body 306′. Thus, during retraction of the suturing device 300 from the foot 314, the link 312 remains engaged with the elongate body 306′, as shown with reference to FIG. 25C. As the elongate body 306′ and the catch 332 retract from the foot 314, the catch 332 pulls the link 312 through the foot 314, also as shown with reference to FIG. 25C. While the catch 332 pulls the link 312, the cuff 310 (not shown) and the suture 302 (not shown) move through the foot 314 in order to enable suturing of an incision.

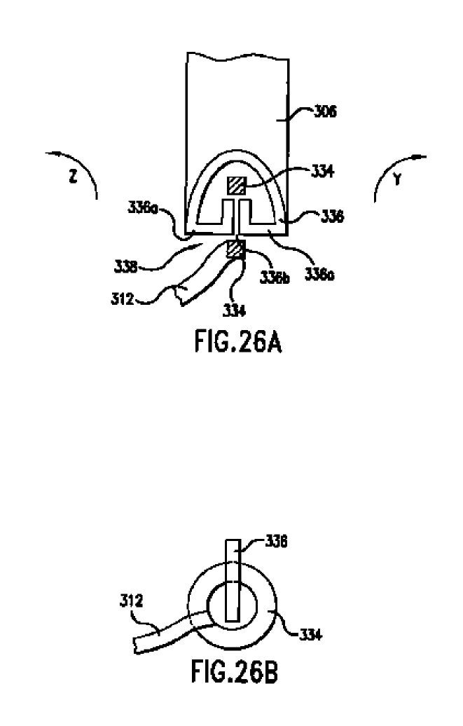

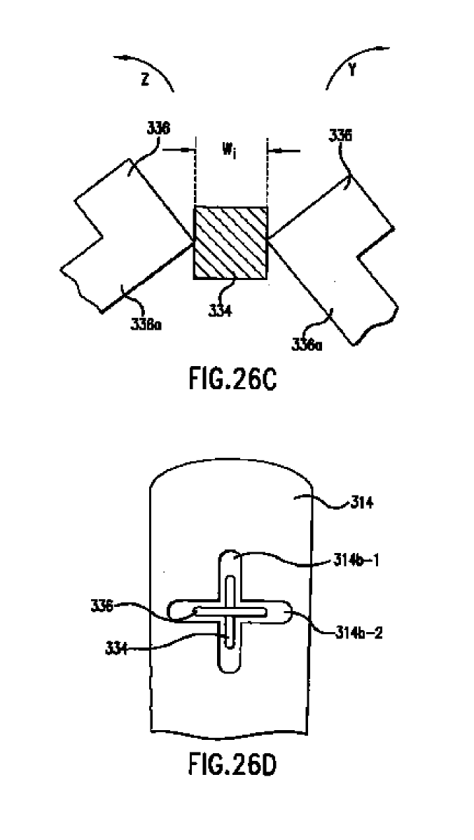

In another embodiment, the suturing device 300 may also employ a clip and ring assembly 338 which couples the elongate bodies 306 and 306′ with the link 312, as shown with reference to FIG. 26A. FIG. 26A illustrates a schematic view of the clip and ring assembly 338 for coupling the elongate bodies 306 and 306′ with the link 312 in accordance with an embodiment of the present invention. The elongate bodies 306 and 306′ include a clip 336 in place of the penetrator tip 308 and the needle tip 308′ where the clip 336 has a configuration as shown with reference to the Figure. The clips 336 include flexible arms 336a and a passageway 336b.

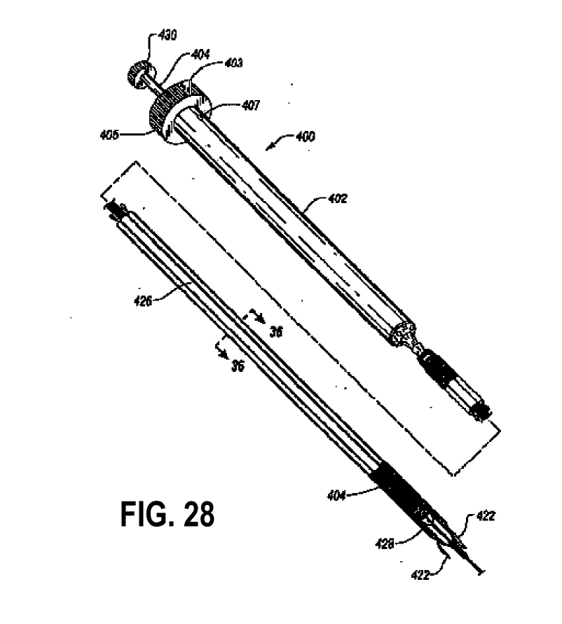

The clip and ring assembly 338 also includes a ring 334 that engages with the clip 336. The link 312 couples with the ring 334 using any suitable technique, such as tying or the like. The ring 334 has a circular configuration as shown with respect to FIG. 26B such that as the elongate bodies 306 and 306′ engage with the foot 314, the clip 336 couples with the ring 334. As the clips 336 engage with the ring 334, the flexible anus 336a flex in a direction indicated by directional arrows Y and Z thereby increasing a width Wi of the passageway 336b in order to allow passage of the ring 334 through the clip 336 as shown with regards to FIG. 28C.

Referring to FIG. 26D, there is shown a top view of the foot 314 where the foot 314 includes cuff pockets 314b-1 and 314b-2. The cuff pocket 314b-1 holds the ring 334 prior to engagement with the clip 336. The cuff pocket 314b-2 is configured such that as the elongate bodies 306 and 306′ enter the foot 314, the clips 336 enter the cuff pocket 314b-2 and engage with the ring 334 as shown with reference to the Figure. Once the clip 336 engages with the ring 334, the clip 336 coupled with the elongate body 306 detaches from the clip 336 while the elongate body 306′ remains engaged with the clip 336. During retraction of the elongate bodies 306 and 306′ from the foot 314, the elongate body 306′ pulls the link 312 and the suture 302 through the foot 314 in order to suture an incision.

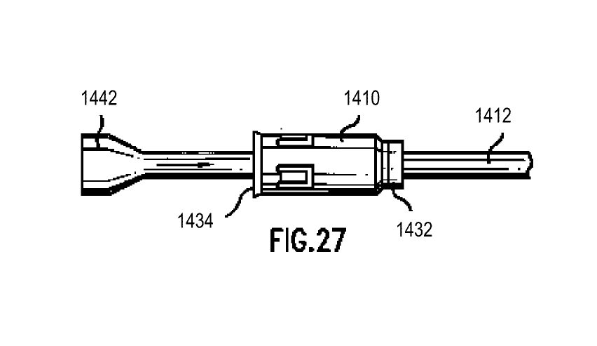

FIG. 27 shows an embodiment of a cuff 1410 and link 1412 assembly that may be provide with the various embodiments of the present invention. Cuff 1411 has a penetrator tip receiving end 1434 and a tapered end 1432. Link 1412 has two ends 1442 (only one shown in FIG. 27). An example of a preferred link material is expanded Polytetrafluoroethylene (ePTFE). PTFE is commonly referred to as Teflon. ePTFE is particularly suited for use as the link material in the vessel closure devices described herein because of its low friction, high strength properties.

To assemble the link and cuff assembly, a length of link material is first threaded through the cuff. The end of the link material extending from the penetrator tip receiving end 1434 of the cuff 1410 is then heated so that it expands. The link is then pull through the cuff 1410 such that the expanded end portion 1442 is seated in the interior tapered end 1432 of the cuff 1410.

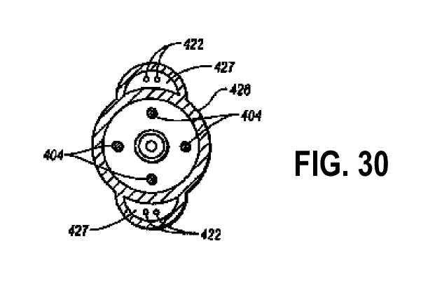

Referring to FIGS. 28-30, a suture applying device 400 in accordance with a further embodiment which is suitable for suturing and sealing of percutaneous vascular puncture site, particularly those made to the femoral artery in a patient's groin, will be described. It will be appreciated, however, that the device of the present invention can be readily adapted for use with punctures made to other hollow body organs and lumens, although it may be necessary to modify the dimensions and other particular aspects of the device to accommodate the different usage environment.

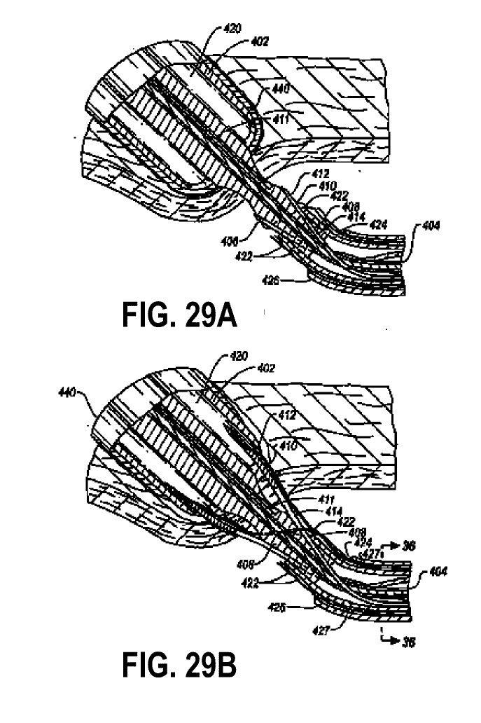

The device 400 comprises a guide body 402 and a needle shaft 404. The guide body 402 includes a guide tip 406 at its distal end, which guide tip includes a plurality of guide channels 408 which receive the proximal ends of needles 410. An aligning arrow 403 is mounted on handle 405 located at the proximal end of the guide body 402. A marker lumen bubble 407 is located below the aligning arrow and serves to indicate when the distal end of the guide body has entered a blood vessel, as described in the embodiment below. An indicator lumen 411 which permits the flow of blood to the marker lumen bubble 407 is illustrated in FIGS. 29A and 29B.

The needles 410 as illustrated comprise a sharpened tip section 412 and an elongate shank portion 414, but may also be manufactured as an integral piece. The shank portion 414 will be sufficiently long so that the needles may be pushed from their butt end by a support holster 428 fixedly attached to the needle shaft 404 in order to advance the needles through the tissue to be sutured and fully through the guide body 402 inserted together with support sheath 440 in the associated tract so that no capture mechanism will be required.

The guide body 402 further includes a plurality of needle lumens 420 which are axially aligned and spaced about the periphery of the guide body. As best seen in FIG. 29B, the needles 410 will enter the distal ends of the lumens 420 as the needles are advanced proximally relative to the guide body.

A flexible needle sheath 426 will be attached to the guide tip 406 of guide body 402. The central lumen of the needle sheath 426 receives a support holster 428 attached to the distal end of the needle shaft 404, as well as the needles 410. As with previous embodiments, the butts of the needles 410 are removably received within the support holster 428. The sheath 426 will be sufficiently long to permit the needles to extend at least 5 cm beyond the distal end of guide body 402.

Prior to use, the suture applying device 400 will be in the configuration illustrated in FIGS. 28 and 29A. That is, the needle shaft 404 will be distally positioned within the guide body 402 and needle sheath 426. In particular, the tips of needles 412 will lie just at the guide tip 406 so that they may be easily advanced through the arterial tissue surrounding the arteriotomy. That is, the tips of the needles will be generally retracted within the guide tip 406. A length of suture 422 is attached to the proximal tips 412 of opposed pairs of needles 410, with the connecting suture being stored in side lumens 427 extending axially along the exterior of the needle sheath 426. As best observed in FIGS. 29A and 29B, the suture 422 extending between one pair of opposed needles is received in a first of the side lumens 427, while the suture extending between the other pair of opposed needles is received in the second of the side lumens. While it would be possible to store the suture 422 in the lumens 420 of the guide body 402 (and thus eliminate the need for side lumens 427), such storage is less preferred since it increases the risk that the suture will become entangled with the needles 410 as they are withdrawn proximally. The use of side lumens 427 greatly simplifies feeding of the suture as the needles 410 are withdrawn.

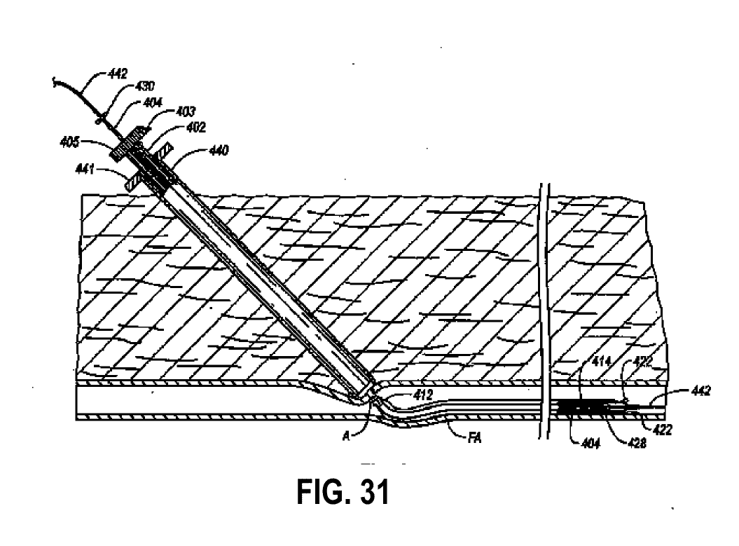

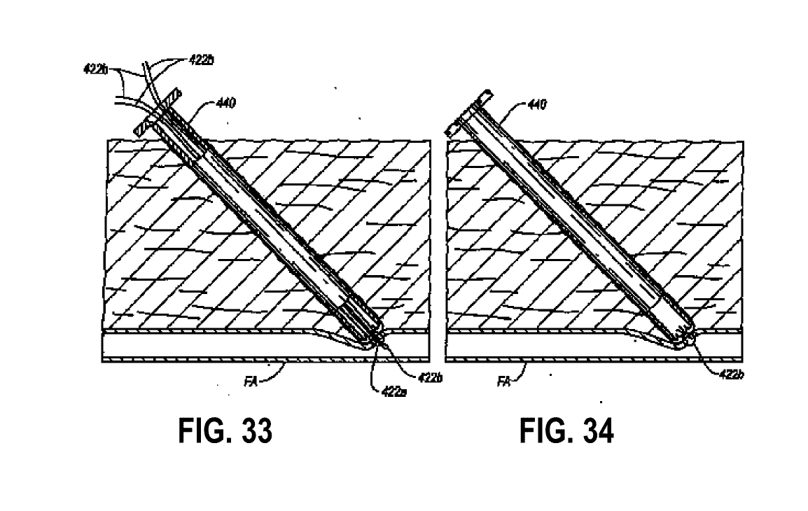

After the guide tip 406 has been passed through the puncture site to be sutured, the needles may then be drawn proximally forward through the tissue to be sutured by drawing proximally on handle 430 at the proximal end of needle shaft 404. The method of the present invention will now be described in more detail with reference to FIGS. 31-34.

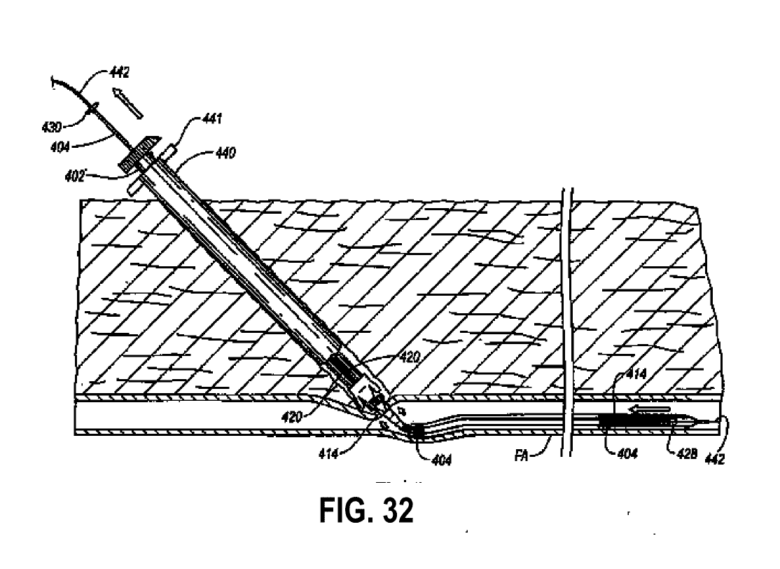

The situation following an interventional or other vascular procedure, where the attending physician is satisfied that the puncture site may be sealed, is illustrated in FIG. 31. A conventional introducer sheath is in place with a guidewire passing into the femoral artery. The conventional introducer sheath is withdrawn after assuring that an appropriate guidewire for the suturing process is in place. The device 400 (including a support sheath 440 which initially covers the ports to the needle lumens 420) will then be introduced over the guidewire, as illustrated in FIG. 31. The needles 410 and sutures 422 mostly encased by flexible needle sheath 426, will be fully advanced into the femoral artery FA past the arterial puncture site A. Handle 441 on support sheath 440 is then partially withdrawn proximally to expose the needle lumens 420 (as shown in FIGS. 29A, 29B, and 32). Handle 430 will then be drawn proximally outward relative to the guide body 402, causing the needles 410 to pass through the superficial wall of the femoral artery FA and into the needle lumens 420, as illustrated in FIGS. 29B and 32. The handle 430 may continue to be drawn proximally (i.e., outward from the patient) in order to continue to pull the needle shaft 404 through the guide body 402. Such movement of the needle shaft 404, in turn, continues to draw the needles 410 outward through the lumens 420 of the guide body 402 until the tips of the needles are exposed. The user may then grasp the needles and continue to draw them out until the suture is available to the user. The guide body 402 may then be withdrawn from the support sheath 440, leaving a portion of the needle sheath 426 still in the puncture site A to maintain hemostasis.

Procedures requiring access to the vessel are performed at this time, e.g., insertion of medical devices typically involving the use of catheters, sometimes referred to as the pre-close technique. For example, following suture positioning, stent graft insertion may be performed through the access point in the blood vessel. A heart valve replacement, such as the SAPIEN transcatheter heart valve (Edwards Lifesciences, Irvine, Calif., USA), may be deployed through this access point. Such procedures may include introducer sheaths having an outer dimension of 5 F-8 F, and may be larger than 8 F, as discussed below.

Following withdrawal of the catheters and/or other medical devices, the suture can then be tied and the knot pushed back down through the support sheath 440. The knot will then only be tightened when the needle sheath is finally withdrawn from the puncture site A.

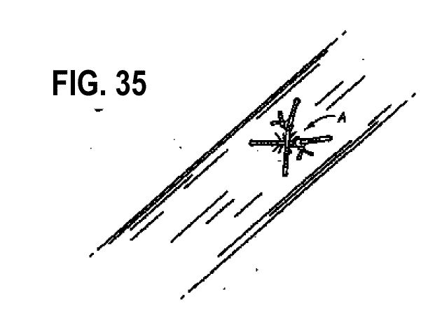

It can be seen that the guide tip 406 deflects the needles radially outward so that the pattern of four needles engages the artery wall in an approximately square pattern about the arteriotomy A. After the sutures are tied and the knots advanced back through the support sheath 440, the resulting pattern of tied suture will appear as in FIG. 35 when viewed towards adventitial surface of the femoral artery FA surrounding the arteriotomy A.

Device 400 has certain advantages over the previous embodiments. Since it is not necessary to capture the needles using an internal capture mechanism, the needles need not have barbs. Such barbless needles will minimize trauma to the arterial tissue around the puncture site A and simplify the procedure. The guide body 402 and guide tip 406 are designed as an integral structure to assure that needles 410 will be precisely centered around the puncture site A, and will very reliably enter the needle lumens 420 in guide body 402. Also, tip 406 will occlude the arteriotomy puncture during the performance of the procedure, providing hemostasis. Moreover, the entire procedure is simplified, with fewer discrete steps being performed. The user need only introduce the device over-the-wire and thereafter draw out the needle shaft to carry the needles through the tissue to be sutured and outward through the guide body, where the suture becomes accessible and may be tied in a conventional manner.

The present invention offers surgeons an automated method for delivering a pre-tied knot to an incision formed in a lumen. The present invention minimizes the problems associated with a surgeon manually delivering a knot to an incision site. Thus, the present invention reduces the time required to accurately and precisely place a suture knot in close proximity to an incision formed in a lumen, thereby decreasing both the overall time a patient spends in surgery and the costs associated with spending time in surgery.

### Example

A clinical trial was performed, which was a prospective, multicenter, randomized, concurrently-controlled trial. Patients with AAA who were suitable candidates for endovascular repair using a graft introduction system and for percutaneous femoral artery closure and who met the prospectively defined inclusion/exclusion criteria were randomized to treatment with a graft system via a totally percutaneous access approach (PEVAR=Test) or via a standard vascular exposure cutdown approach (SEVAR=Control). PEVAR patients had their femoral artery access sites closed using, e.g., the Perclose ProGlide® suture-mediated closure system (“ProGlide”) (Abbott Vascular, Inc., Redwood City, Calif.), substantially identical to the vessel closure device 10 described herein. Prior to the randomization of the first patient at each investigational site, a minimum of two patients were treated in a roll-in phase at the investigational site. Roll-in patients underwent the same treatment and follow-up as the randomized patients.

The PEVAR trial includes the Independent Access Site Closure Study which is a set of analyses designed to evaluate the safety and effectiveness of the vessel closure device using the pre-close technique to percutaneously close ipsilateral femoral artery access sites up to 21 F sheath size. The primary analysis is based on a non-inferiority hypothesis test to demonstrate the vessel closure arm is non-inferior to the SEVAR arm. Data from the ProGlide (n=50) and SEVAR (n=50) arms, are described herein.

Methods: All patients underwent pre-procedure assessments prior to enrollment in the trial. The protocol requires clinical assessments prior to discharge, at one month and six months. An independent clinical events committee adjudicated potential endpoint events of both major and minor ipsilateral access site vascular complications. The enrollment has been completed and follow-up will continue until all patients complete the six-month visit. The following assessments were required at pre-discharge, one month, and six months:

Medication review (one and six months only); physical exam, including overall health and physical assessment, lower extremity sensorimotor exam and access site assessment; serum creatinine, blood urea nitrogen, hematocrit and hemoglobin; ABI; contrast-enhanced CT scan of the abdomen and pelvis (one month only); bilateral femoral duplex ultrasound (pre-discharge and six month only); SF-36 QOL (1 and 6 months only); pain scale; and adverse events.

### Results of the Randomized Data

Patient Demographics: In general, baseline demographics were comparable between the ProGlide and the SEVAR patients. There was a difference in age between the ProGlide and SEVAR arms (69.9±6.6 vs. 73.2±8.8) that did not appear to affect the overall study outcome, based on additional adjusted analysis.

Primary Endpoint: The primary endpoint for the trial is the major ipsilateral access site vascular complication rate at 30 days for patients treated percutaneously (PEVAR ProGlide arm) compared to that of patients treated using standard surgical vascular access (SEVAR group).

Major ipsilateral access site vascular complications are a composite of the following events: access site vascular injury requiring surgical repair, angioplasty, or ultrasound-guided compression, or thrombin injection; new onset lower extremity ischemia that is attributed to arterial access or closure causing a threat to the viability of the limb and requiring surgical or additional percutaneous intervention; access site-related bleeding requiring transfusion; access site-related infection requiring intravenous antibiotics or a prolonged hospitalization; access site-related nerve injury that is permanent or requires surgery.

The study results show that at 30 days, ProGlide patients had a 6.0% (3/50) major ipsilateral access site vascular complication rate vs. the SEVAR patients who had a 10% (5/50) major ipsilateral access site vascular complication rate. The non-inferiority test for the primary endpoint revealed a p value=0.0048 and resulted in the rejection of the null hypothesis, demonstrating that ProGlide is non-inferior to SEVAR in the closure of femoral artery access sites up to 21 F sheath size (Table 1).

TABLE 1

| | ProGlide | SEVAR | p- |
|---|---|---|---|
| | N = 50 | N = 50 | value |
| Major Ipsilateral Access | 6.0% (3/50) | 10.0% (5/50) | 0.0048 |
| Site Vascular Complica- | [1.3%, 16.5%] | [3.3%, 21.8%] | |
| tion at 30 days [95% | | | |
| Confidence Interval] | | | |

Table 1 represents the results of a non-inferiority test for primary endpoint-per subject analysis for a modified intent-to-treat population (defined as all patients who were randomized and treated) for ProGlide vs. SEVAR. The 95% confidence interval was the Clopper-Pearson exact confidence interval. The p-value represents a one-sided p-value and 95% confidence interval for non-inferiority test by using asymptotic test statistics with non-inferiority margin of 10%.

Select Secondary Endpoints: In the trial, the following select secondary endpoints were also evaluated:

Procedure time was defined as elapsed time from the first skin break to final closure (skin to skin time).

Minor ipsilateral access site complications included minor ipsilateral access site vascular complications and narcotic analgesic use for ipsilateral access site pain at 30 days.

Minor ipsilateral access site vascular complications included: Access site pseudoaneurysm or AV fistula documented by ultrasound; Access site hematoma ≧6 cm; Post-discharge access site-related bleeding requiring >30 minutes to re-achieve hemostasis; Lower extremity arterial emboli or stenosis that is attributed to arterial access or closure; Deep vein thrombosis; Access site-related vessel laceration; Transient access site-related nerve injury; Access site wound dehiscence; Access site related lymphocele; and Localized access site infection treated with intramuscular or oral antibiotics.

Additional secondary endpoints considered include: time to actual hospital discharge defined as elapsed time from sheath removal to actual physical discharge from the hospital; time to ambulation defined as elapsed time between sheath removal and time when the patient stands and walks at least 20 feet without re-bleeding; ipsilateral pain score at pre-discharge; time to hemostasis for the ipsilateral access site defined as elapsed time from sheath removal to first observed cessation of CFA bleeding (excluding cutaneous or subcutaneous oozing); closure device success defined as successful achievement of index procedure ipsilateral access site hemostasis with percutaneous closure without surgical intervention; and ipsilateral access site closure success defined as successful achievement of hemostasis with percutaneous closure devices and without surgical intervention and freedom from major ipsilateral access site vascular complications within 48 hours of the index procedure or hospital discharge, whichever occurs first.

As shown in Table 2, the ProGlide arm had a 25% shorter procedure time than the SEVAR arm (106.5±44.9 vs. 141.1±73.4, p=0.0076). Although there were no statistically significant differences in the minor ipsilateral access site complications, time to actual hospital discharge, time to ambulation and ipsilateral pain score at pre-discharge between the ProGlide and SEVAR arms, fewer patients in the ProGlide arm required narcotic analgesic use, had minor ipsilateral access site complications and ProGlide patients had slightly shorter time to actual discharge. In the ProGlide arm, the time to hemostasis for the ipsilateral access site was 57% shorter than in the SEVAR arm (9.8±17 vs, 22.7±22.9 minutes, 95% CI of the difference [−21.1, −4.7]). In addition, the ProGlide arm achieved a high device success rate and high access site closure success rate at 96% and 94%, respectively.

TABLE 2

| | | | | Superiority |
|---|---|---|---|---|
| | ProGlide | SEVAR | Difference | Test |
| Secondary Endpoints | N = 50 | N = 50 | (95% CI) | p-value |
| Procedure Time (minutes) | 106.5 ± 44.9 (50) | 141.1 ± 73.4 (50) | −34.7 | 0.0076 |
| [95% Confidence Interval] | [93.7, 119.2] | [120.3, 162.0] | [−58.9, −10.4] | |
| Minor Ipsilateral Access Site | 22.0% (11/50) | 30.0% (15/50) | −8.0% | 0.4954 |
| Complications at 30 days | [11.5%, 36.0%] | [17.9%, 44.6%] | [−25.1%, 9.1%] | |
| [95% Confidence Interval] | | | | |
| Minor Ipsilateral Access Site | 4.0% (2/50) | 8.0% (4/50) | −4.0% | — |
| Vascular Complications at 30 days | [0.5%, 13.7%] | [2.2%, 19.2%] | [Assumptions | |
| [95% Confidence Interval] | | | not met] | |
| Narcotic Analgesic Use for Ipsilateral | 18.0% (9/50) | 28.0% (14/50) | −10.0% | — |
| Access Site Pain at 30 days | [8.6%, 31.4%] | [16.2%, 42.5%] | [−26.4%, 6.4%] | |
| [95% Confidence Interval] | | | | |
| Time to Actual Hospital Discharge | 31.4 ± 16.9 (50) | 45.7 ± 59.9 (48) | −14.3 | — |
| (hours) | [26.6, 36.2] | [28.3, 63.1] | [−32.3, 3.7] | |
| [95% Confidence Interval] | | | | |
| Time to Ambulation (hours) | 17.8 ± 7.2 (50) | 20.5 ± 16.9 (48) | −2.7 | — |
| [95% Confidence Interval | [15.7, 19.9] | [15.6, 25.5] | [−8.0, 2.5] | |
| Ipsilateral Pain Scale Score at Pre- | 2.1 ± 2.2 (50) | 2.6 ± 2.4 (49) | −0.5 | — |
| Discharge | [1.5, 2.7] | [1.9, 3.3] | [−1.4, 0.4] | |
| [95% Confidence Interval] | | | | |
| Time to Hemostasis for Ipsilateral | 9.8 ± 17.0 (50) | 22.7 ± 22.9 (47) | −12.9 | — |
| Access Site (minutes) | [5.0, 14.7] | [16.0, 29.4] | [−21.1, −4.7] | |
| [95% Confidence Interval] | | | | |
| Closure Device Success | 96.0% (48/50) | N/A | N/A | — |
| [95% Confidence Interval] | [86.3%, 99.5%] | | | |
| Access Site Closure Success | 94.0% (47/50) | N/A | N/A | — |
| [95% Confidence Interval] | [83.5%, 98.7%] | | | |

The 95% Confidence Interval for Procedure Time, Time to Actual Hospital Discharge, Time to Ambulation, Ipsilateral Pain Scale Score at Pre-Discharge, and Difference (95% CI) were determined by normal approximation. The 95% Confidence Interval for Minor Ipsilateral Access Site Complications at 30 days, Minor Ipsilateral Access Site Vascular Complications at 30 days, Narcotic Analgesic Use for Ipsilateral Access Site Pain at 30 days, Time to Hemostasis for Ipsilateral Access Site, Closure Device Success, and Access Site Closure Success were determined by Clopper-Pearson exact confidence interval. The Superiority Test p-value for the for procedure time and the Time to Hemostasis for Ipsilateral Access Site was determined by two-sample t-test, pre-specified hypothesis test based hierarchical test procedure. The Superiority Test p-value for the Minor Ipsilateral Access Site Complications at 30 days was determined by Fisher's Exact Test, pre-specified hypothesis test based hierarchical test procedure. The Minor Ipsilateral Access Site Complications at 30 days was a composite endpoint including minor Ipsilateral Access site vascular complications and narcotic analgesic use for Ipsilateral access site pain at 30 days. For the Difference (95% CI) of the Minor Ipsilateral Access Site Complications at 30 days, there was an insufficient sample size or small frequency in the numerator for the validity of normal approximation assumption

Adverse events related to major and minor ipsilateral access site vascular complications that occurred within the first 30 days are listed in Table 3, which includes only the subject's first occurrence of each event.

TABLE 3

| | ProGlide | SEVAR |
|---|---|---|
| | N = 50 | N = 50 |
| Major Ipsilateral Access site Vascular Complications at 30 Days | 6.0% (3/50) | 10.0% (5/50) |
| Access site vascular injury requiring surgical repair, angioplasty, or ultrasound- | 2.0% (1/50) | 2.0% (1/50) |
| guided compression, or thrombin injection | | |
| New onset lower extremity ischemia that is attributed to arterial access or | 4.0% (2/50) | 4.0% (2/50) |
| closure causing a threat to the viability of the limb and requiring surgical or | | |
| additional percutaneous intervention | | |
| Access site-related bleeding requiring transfusion | 2.0% (1/50) | 4.0% (2/50) |
| Access site-related infection requiring intravenous antibiotics or a prolonged | 0.0% (0/50) | 0.0% (0/50) |
| hospitalization | | |
| Access site-related nerve injury that is permanent or requires surgery | 0.0% (0/50) | 2.0% (1/50) |
| Minor Ipsilateral Access Site Vascular Complications at 30 days | 4.0% (2/50) | 8.0% (4/50) |
| Access site pseudoaneurysm or AV fistula documented by ultrasound | 0.0% (0/50) | 0.0% (0/50) |
| Access site hematoma ≧ 6 cm | 0.0% (0/50) | 2.0% (1/50) |
| Post-discharge access site-related bleeding requiring >30 minutes to re-achieve | 0.0% (0/50) | 0.0% (0/50) |
| hemostasis | | |
| Lower extremity arterial emboli or stenosis that is attributed to arterial access or | 4.0% (2/50) | 4.0% (2/50) |
| closure | | |
| Deep vein thrombosis | 0.0% (0/50) | 0.0% (0/50) |
| Access site-related vessel laceration | 0.0% (0/50) | 0.0% (0/50) |
| Transient access site-related nerve injury | 0.0% (0/50) | 2.0% (1/50) |
| Access site wound dehiscence | 0.0% (0/50) | 0.0% (0/50) |
| Access site related lymphocele | 0.0% (0/50) | 0.0% (0/50) |
| Localized access site infection treated with intramuscular or oral antibiotics | 0.0% (0/50) | 0.0% (0/50) |

Clinical Data from the Roll-in Phase: There were 22 patients treated in the ProGlide roll-in phase of the PEVAR trial. The mean age of this treatment group was 71.1±6.9 years. The major ipsilateral access site vascular complication rate was 4.5% (1/22). The mean procedure time was 118.2±43.4 minutes, and the average time to hemostasis was 7.7±6.8 minutes for the roll-in phase. Additionally, the device success rate and the access site closure success rate were both 95.5% respectively. These results are comparable to the ProGlide arm in the randomized phase and substantiate the safety and effectiveness of the ProGlide devices.

### Example

The Closer IDE clinical trial was designed as an equivalency trial for the 30-day primary combined safety endpoint of freedom from major complications and a primary efficacy endpoint of time to discharge when compared to the control group (STAND II Trial). The study prospectively examined the safety and effectiveness of femoral artery closure using the Closer 6 F SMC Device, substantially identical to the vessel closure device 10, 10′, 100 and 300 described herein, following interventional catheterization procedures using 5 F to 8 F sheaths. Two hundred twenty five (225) patients were enrolled in post-close arm and one hundred sixty (160) patients were enrolled in the pre-close arm of the Closer IDE Trial. In the post-close arm, the deployment of the Closer device occurred at the end of the catheterization procedure. In the pre-close arm, the Closer device was deployed in two steps with suture delivery at the beginning of the catheterization procedure with knot tying and knot delivery occurring at the end of the procedure.

Procedural success was achieved in 223 patients (99.1%) in the post-close arm and 158 patients (98.8%) in the pre-close arm. Time to discharge was 28.9±22.7 hours and 30.1±33.9 hours for the post-close and pre-close patients respectively. The secondary endpoint of time to hemostasis was 10.9±42.0 minutes and 8.2±51.0 minutes for the post-close and pre-close patients respectively, versus 7.9±6.4 hours for the control group patients, p<0.0001, and the secondary endpoint of time to ambulation was 4.7±7.1 hours and 6.5±11.4 hours for the post-close and pre-close patients respectively.

Device success was 92.0% (207/225 patients) in the post-close arm and 89.4% (143/160 patients) in the pre-close arm. Failure to deploy the Closer occurred in 17 (7.6%) patients in the post-close arm and 15 (9.4%) patients in the pre-close arm.

A major complication was defined as surgical repair of vascular injury, ultrasound-guided compression, groin related transfusion, or groin related infection requiring IV antibiotics and extended hospitalization. The primary safety endpoint was the combined rate of major complications at 30 days. For the post-close arm, one patient received a blood transfusion subsequent to a retroperitoneal bleed. Another patient underwent surgical repair of a vascular injury and received a blood transfusion subsequent to the intervention. Both patients were free of symptoms at time of follow up. For the pre-close arm, one patient developed a hematoma >6 cm as a result of insufficient hemostasis. Subsequently, the patient required vascular surgery to repair the femoral artery and received blood transfusions intraoperatively. The second patient received IV antibiotic therapy for a local infection that presented post discharge. Both patients reported no further sequelae at time of follow-up.

The incidence of vascular complication other than major was a secondary safety endpoint of the study and in the post-close arm consisted of one (0.4%) false aneurysm, one (0.4%) infection requiring IM and PO antibiotics, two (0.9%)>6 cm hematomas, and two (0.9%) retroperitoneal bleeds not requiring intervention. For the pre-close arm, the incidence of vascular complication other than major consisted of one (0.6%)>6 cm hematoma and one (0.6%) groin infection requiring PO antibiotics. All patients were free of symptoms at time of follow up. The results of the effectiveness measures are summarized in Table 4.

TABLE 4

| | The Closer IDE | The Closer IDE |
|---|---|---|
| | Trial Post-Close | Trial Pre-Close |
| Effectiveness Measures | Patients | Patients |
| Treated patients (per | n = 225 | n = 160 |
| event) | | |
| Procedural success | 223 (99.1%) | 158 (98.8%) |
| Device success | 207 (92.0%) | 143 (89.4%) |
| Device failure | 17 (7.6%) | 15 (9.4%) |
| Device malfunction | 16 (7.1%) | 14 (8.8%) |
| Device complication | 1 (0.4%) | 1 (0.6%) |
| Time to Hemostasis (mins) | n = 224 | n = 160 |
| mean ± SD | 10.9 ± 42.0 | 8.2 ± 51.0 |
| (min. max.) | (1.0, 324.0) | (0.1, 639.0) |
| Median | 3.0 | 1.5 |
| [quartiles] | [2.0, 5.0] | [0.0, 5.0] |
| Time to Ambulation (hrs) | n = 225 | n = 160 |
| mean ± SD | 4.7 ± 7.1 | 6.5 ± 11.4 |
| (min. max.) | (0.1, 71.4) | (0.05, 100.9) |
| Median | 2.4 | 2.2 |
| [quartiles] | [1.6, 4.5] | [1.2, 5.0] |
| Time to Discharge (hrs) | n = 225 | n = 160 |
| mean ± SD | 28.9 ± 22.7 | 30.1 ± 33.9 |
| (min. max.) | (2.2, 240.2) | (2.7, 292.6) |
| Median | 24.4 | 22.5 |
| [quartiles] | [22.0, 27.2] | [20.2, 26.1] |

The number of patients listed under effectiveness measures is less than the total patients studied due to missing data for some patients. Device success is defined as acute success using the device only or the device+adjunctive (non-arterial) compression.

Thus, the vessel closure system reduced the time to hemostasis, ambulation (10 feet) and discharge in patients who had undergone diagnostic or interventional catheterization procedures without complicating clinical conditions.

ADVERSE EVENTS: The Closer IDE Trial was designed as a multi-center, multi-operator, prospective registry enrolling patients in a post-close arm and a pre-close arm. The post-close arm studied the use of a 6 F system following interventional procedures using 5 F to 6 F sheaths. The pre-close arm studied the use of the 6 F system following interventional procedures using 7 F to 8 F sheaths utilizing the pre-close technique. The pre-specified analysis of the primary safety endpoint of the IDE Trial was the incidence of the combined rate of major complications at 30 days of patients undergoing interventional catheterization procedures. Post treatment, ultrasound evaluations were performed 0 to 15 days post discharge to verify detection of clinical complications. Two major complications were reported in each of the post-close and pre-close arms of the trial. Neither of the two major complications reported in the post-close or pre-close arms were considered unanticipated events. No delayed major hemorrhagic events were reported despite early ambulation and early discharge of the patients with the closure device. The adverse events that were observed during the trial are reported in Table 4.

TABLE 4

| | The Closer IDE | The Closer IDE |
|---|---|---|
| | Trial Post-Close | Trial Pre-Close |
| Safety Measures, n (percent) | Patients | Patients |
| Treated patients (per event) | n = 225 | n = 160 |
| Device Failure | 17 (7.6%) | 15 (9.4%) |
| Surgical repair* | 1 (0.4%) | 1 (0.6%) |
| U/S guided compression* | 0 (0.0%) | 0 (0.0%) |
| Transfusion* | 2 (0.9%) | 1 (0.6%) |
| Infection requiring IV Abx* | 0 (0.0%) | 1 (0.6%) |
| Hematoma ≧ 6 cm | 2 (0.9%) | 1 (0.6%) |
| AV-fistula | 0 (0.0%) | 0 (0.0%) |
| Pseudoaneurysm | 1 (0.4%) | 0 (0.0%) |
| Vascular narrowing | 0 (0.0%) | 0 (0.0%) |
| Infection requiring IM\PO Abx | 1 (0.4%) | 1 (0.6%) |
| Retroperitoneal bleed | 2 (0.9%) | 0 (0.0%) |
| Incidence of Complications (per | | |
| patient) | | |
| Any complication^{¶} | 6 (2.7%) | 3 (1.9%) |
| Major complication^{¶} | 2 (0.9%) | 2 (1.2%) |
| No major complication | 223 (99.1%) | 158 (98.8%) |

No groin or device related deaths were reported in the trial among the post-close or pre-close study patients. Other adverse events potentially associated with the use of the closure system were reported as an underlying event or did not occur during the clinical study. These include: deep vein thrombosis, infection extending hospitalization, late bleeding, wound dehiscence, vessel laceration, local pulse deficits or ischemia, embolization, transitory local irritation, nerve injury and vascular spasm. In addition, polyester surgical sutures elicit a minimal acute inflammatory reaction in tissues, followed by gradual encapsulation of the suture by fibrous connective tissue. Polyester surgical sutures are not absorbed, nor is any significant change in tensile strength known to occur in vivo.

### Clinical Procedure

The following describes the techniques used herein:

Arterial Site and Puncture Considerations: Before inserting the access needle, ultrasound guidance can be used to visualize the common femoral artery or fluoroscopy can be used to visualize the femoral head. When using the femoral head as a reference point, the middle of the femoral head is targeted as the puncture site. A femoral angiogram through the introducer sheath (or procedural sheath) can be performed to verify that the access site is in the common femoral artery before anticoagulants are given. The anterior wall of the common femoral artery can be punctured at an angle of approximately 45 degrees, avoiding side wall or posterior wall femoral artery punctures.

Prior to deployment of the vessel closure device, a femoral angiogram can be performed to evaluate the femoral artery site for vessel size, calcium deposits, tortuosity, and for disease or dissections of the arterial wall to avoid device cuff misses (device needles not engaging with the cuffs) and/or posterior wall suture placement and possible ligation of the anterior and posterior walls of the femoral artery. Angiographically verification can be performed to ensure that the puncture is on the anterior wall of the common femoral artery. The puncture should be proximal to the bifurcation of the superficial femoral artery and the profunda femoris branch and distal to the inferior margin of the inferior epigastric artery.

Deployment sequence to close the access site of a catheterization procedure performed through a 5 F to 8 F sheath size: In some embodiments, a 0.038″ (or smaller) guidewire GW is placed through the procedural (or introducer) sheath. The procedural sheath is removed while applying pressure on the groin to maintain hemostasis. The vessel closure device is backloaded over the guidewire until the guidewire exit port of the device sheath is just above the skin line. The guidewire is removed before the exit port crosses the skin line. The vessel closure device is further advanced just until brisk pulsatile flow of blood is evident from the marker lumen. The device is then positioned at a 45-degree angle. The foot is deployed by lifting the lever on top of the handle only if brisk pulsatile flow of blood (“mark”) is evident from the marker lumen.

The vessel closure device is then gently pulled back to position the foot against the arterial wall. If proper position of the foot has been achieved, tactile sensation will be felt and blood marking will cease or be significantly reduced to a slight drip. If marking does not stop or significantly change, the angiogram can be evaluated for femoral artery size, calcium deposits, tortuosity, disease and for location of the puncture (ensure footplate is not in bifurcation or side branch). The device can be repositioned to stop blood marking, the wire can be reinserted, the device can be removed to hold manual compression, a new sheath can be inserted.

While maintaining vessel closure device position, it is stabilized with the operator's free hand (the one not used to deploy the device) to maintain the gentle retraction and to ensure the vessel closure device does not twist or move forward during deployment. The operator's other hand is used to deploy needles by pushing on the plunger assembly until contact of the collar of the plunger with the proximal end of the body is visually confirmed.

Using the operator's thumb as a fulcrum on the handle, the needles can be gently disengaged by pulling the plunger assembly back and completely removing the plunger and needles from the body of the vessel closure device. One suture limb will be attached to the anterior needle. The posterior needle will be free of suture. The plunger should be pulled back until the suture is taut, which confirms that all the suture as been fully refracted from the body of the device.

A suture-trimming mechanism located on the handle can be used to cut the suture from the anterior needle distal of the link. The device can the be relaxed, and the foot returned to its original position by pushing the lever down to the body of the device. The vessel closure device is withdrawn until the guidewire port exits the skin line.

The suture adjacent to the device sheath is grasped, and the suture ends are pulled through the distal end of the proximal guide. The rail suture limb is the longer of the two suture limbs, and is used to advance the knot. The shorter, non-rail suture limb is used to lock the knot.

If the operator chooses to maintain wire access, the guidewire is reinserted after exposing the guidewire port at skin level and after the sutures have been harvested from the distal guide but before removing the device for knot advancement. The following steps are performed when closing over the wire:

There should be sufficient guidewire exposed out of the guidewire exit port before removing the device. The rail limb of suture should be wrapped around he operator's left index finger, low close to skin level. The vessel closure device is removed with the right hand, while maintaining an adequate length of guidewire inside the artery. This allows placement of another vessel closure device or a sheath in the event that hemostasis is not obtained.

While removing the device with the right hand, the operator simultaneously advances the knot to the arteriotomy by applying slow, consistent increasing tension to the rail suture limb, keeping the suture coaxial to the tissue tract.

If bleeding is controlled, the operator can then remove the guidewire. Next, a snared knot pusher or a suture trimmer is used, as described below, to advance and tighten the knot until complete closure is achieved. The security of the knot can be confirmed by having the patient cough and/or bend his/her leg. Additionally, patients may be able to move freely in bed without head of bed or leg restrictions if the close is successful.

The suture can be placed around the arteriotomy at the beginning of the procedure and knot advancement can be placed on hold until the procedure is complete. The steps described below are performed when using the pre-close technique:

After completing the step of withdrawing the vessel closure device until the guidewire port exits the skin line described above, the device is slightly rotated until the operator can see the two suture limbs in the bend of the distal guide. The sutures are grasped adjacent to the sheath. While holding the two suture limbs together, both suture ends are pulled through the distal end of the proximal guide.

A shodded hemostat or clamp can be placed to hold the two suture limbs together at the distal end of the non-rail suture limb (shortest limb). To prevent knot advancement or locking of the knot, care must be taken not to pull on the individual suture limbs until the clamp is securely holding the two limbs together. After securing the suture limbs and before inserting the procedural sheath, the clamp can be pulled until the suture is taut to remove any suture slack from the tissue tract.

Procedures requiring access to the vessel are performed at this time, e.g., insertion of medical devices typically involving the use of catheters. For example, stent graft insertion may be performed through the access point in the blood vessel. A heart valve replacement, such as the SAPIEN transcatheter heart valve (Edwards Lifesciences, Irvine, Calif., USA), may be deployed through this access point. Such procedures may include introducer sheaths having an outer dimension of 5 F-8 F, and may be larger than 8 F, as discussed below.

At the end of the catheterization, the guidewire can be reinserted into the procedural sheath. Knot advancement to close the arteriotomy will resume starting with the steps for closing over the wire.

Knot Advancement:

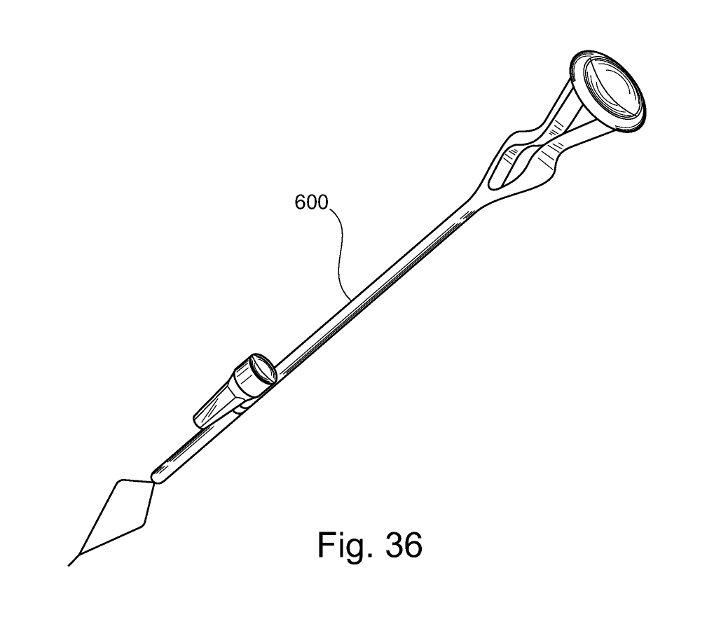

In one embodiment, a snared knot pusher 600 (see FIG. 36) is used to advance the suture knot. Approximately 2 cm of the rail (longer) limb of the suture is placed into the snare at the distal end of the knot pusher. The suture is loaded into the knot pusher by pulling the snare through the tip of the knot pusher.

The rail suture limb (longer limb) is securely wrapped around the operator's left forefinger, low close to skin level. The suture should not be tightened around the sheath. The device or the arterial sheath (if the device was deployed at the beginning of the catheterization procedure) is removed from the artery, while simultaneously pulling gently on the rail limb. The suture is maintained coaxial to the tissue tract. With the rail suture limb securely wrapped around the operator's left forefinger, and held coaxial to the tissue tract, the suture limb can be pulled with slow, consistent increasing tension while advancing the knot pusher into the tissue tract with the operator's right hand until the knot reaches the arterial surface.

The knot pusher can be placed under the operator's left thumb to assume a single-handed position and to complete knot advancement by slow, consist increasing tension on the left forefinger until the rail suture is taut (guitar string tightness).

With the knot pusher held in position and tension maintained on the rail limb, the knot is tightened by gently pulling on the non-rail (shorter) suture limb keeping it coaxial to the tissue tract.

Hemostasis of the access site is achieved when the knot is fully advanced to the arterial surface, the slack is gently pulled from the knot with the non-rail limb while the knot pusher holds tension on the rail limb of the suture, and the tissue is in complete apposition.

The knot pusher can then be removed from the tissue tract, tension relaxed on the suture, and testing for hemostasis performed by having the patient cough or bend his/her leg.

The security of the knot can be confirmed by having the patient cough and/or bend his/her leg. Using the suture trimmer 610 (described below) or a new, sterile scalpel or scissor, the trailing suture limbs can be cut below the skin. Additionally, patients may be able to move freely in bed without head of bed or leg restrictions if the close is successful. If hemostasis cannot be achieved, apply manual compression until hemostasis is achieved.

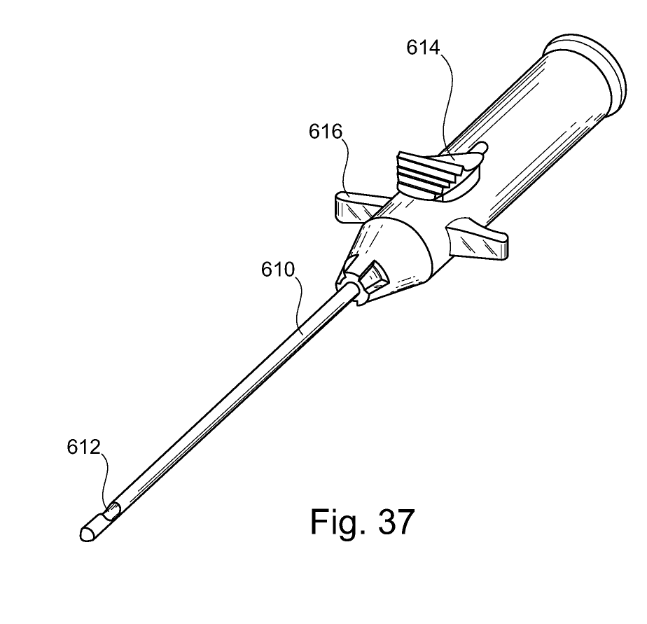

In one embodiment, the suture trimmer 610 (see FIG. 37) is used to advance the suture knot. The rail limb of the suture is securely wrapped around the left forefinger of the operator, low close to the skin.

The suture should not be tightened around the sheath. The vessel closure device or the arterial sheath (if the vessel closure device was deployed at the beginning of the catheterization procedure) is removed from the artery, while simultaneously pulling gently on the rail limb. The suture is maintained coaxial to the tissue tract.

With the rail (longer) suture limb securely wrapped around the operator's left forefinger, the rail limb is placed into the suture trimmer 610 utilizing the following steps: the thumb knob 614 on the suture trimmer is retracted with the operator's right hand. The suture trimmer is placed under the suture limb making an “x” or a “cross” between the suture limb and the mid-point of the suture trimmer. The operator slides the suture trimmer back to load the suture into the suture gate 612 located at the distal end of the suture trimmer. Keeping the thumb knob 614 retracted, the suture trimmer is turned coaxial to the suture and then the thumb knob 614 is released to capture the suture in the suture gate 612. Once the suture is loaded correctly, the suture trimmer should slide easily within the suture trimmer.

With the suture limb and suture trimmer coaxial to the tissue tract, the knot can be moved to the arterial surface by advancing the suture trimmer with the operator's right hand while placing slow, consistent increasing tension on the rail suture with the left forefinger. The suture trimmer and suture limbs should always remain coaxial to the tissue tract. The thumb knob should be at 12 o'clock (facing the ceiling), and the suture trimmer should not be rotated during advancement.

With the rail (longer) suture limb securely wrapped around the left forefinger, the suture trimmer is placed under the left thumb of the operator to assume a single-handed position and complete knot advancement with slow, consistent increasing tension until the suture is taut (guitar string tightness).

With the suture trimmer in place and the suture taut, the knot can be tightened by gently pulling the non-rail (shorter) suture limb, keeping it coaxial to the tissue tract.

Hemostasis of the access site can be achieved when the knot is fully advanced to the arterial surface, the slack is gently pulled from the knot with the non-rail limb while the suture trimmer holds tension on the rail limb of the suture, and the tissue is in apposition.

The suture trimmer can then be removed from the tissue tract, relaxing tension on the suture, and testing for hemostasis by having the patient cough or bend his/her leg. If hemostasis has not been achieved, the single-handed position can be continued for 20 seconds, or until hemostasis is achieved. The knot can be secured again by gently pulling on the non-rail suture limb. The security of the knot can be confirmed by having the patient cough and/or bend his/her leg. Additionally, patients may be able to move freely in bed without head of bed or leg restrictions if the close is successful.

Once hemostasis is achieved, the suture trimmer is used to trim the sutures below the skin. While holding both suture limbs together and pulled taut, both suture limbs are loaded into the suture trimmer as described above and the suture trimmer is advanced to the arterial surface. The sutures are trimmed by pulling back on the trimming lever 616. The operator maintains the trimming lever pulled back while removing the suture trimmer and trimmed suture limbs from the tissue tract. If only one suture limb has been loaded and trimmed, the same technique is repeated on the other suture limb. If hemostasis cannot be achieved, manual compression can be applied until hemostasis is achieved.

The following instructions describe the deployment sequence for closing the access site of an interventional catheterization procedure performed through 8.5 F to 21 F sheath size. The pre-close technique using at least two devices is used when closing sheath sizes from 8.5 F to 21 F.

In some embodiments, a 0.038″ (or smaller) guidewire GW is advanced through the access point through an introducer sheath. The guidewire is advanced to location distal (downstream) from the affected site, e.g., AAA. The introducer sheath is removed while applying pressure on the groin to maintain hemostasis. The first of two vessel closure devices is backloaded over the guidewire until the guidewire exit port of the device sheath is just above the skin line. The guidewire is removed before the exit port crosses the skin line. The vessel closure device is advanced into the blood vessel just until brisk pulsatile flow of blood is evident from the marker lumen. The device lever is positioned facing the ceiling (12 o'clock).

The first vessel closure device is rotated approximately 30 degrees towards the patient's right side (approximately 10 o'clock). The device is positioned at a 45-degree angle to the arterial lumen (see, e.g., FIGS. 13B-C and 15A). The foot is deployed by lifting the lever on top of the handle (FIGS. 13C and 15B) only if brisk pulsatile flow of blood (“mark”) is evident from the marker lumen.

The device is gently pulled back to position the foot against the arterial wall. If proper position of the foot has been achieved, a tactile sensation will be felt by the operator, and blood marking will cease or be significantly reduced to a slight drip. If marking does not stop or significantly change, the angiogram can be evaluated for femoral artery size, calcium deposits, tortuosity, disease and for location of the puncture (e.g., ensuring the footplate is not in bifurcation or a branch vessel of the common femoral artery). The device is repositioned to stop blood marking (maintaining the 30 degree rotation), or the wire reinserted and the situation evaluated before continuing the procedure.

While maintaining the device position, the operator can stabilize the device with the free hand (e.g., the one not used to deploy the device) to maintain gentle retraction and to ensure the device doesn't twist or move forward during deployment. The operator's other hand is used to deploy the needles by pushing on the plunger assembly until visually confirmation that the collar of the plunger is making contact with the proximal end of the body. (See, e.g., FIGS. 13D and 15B.)

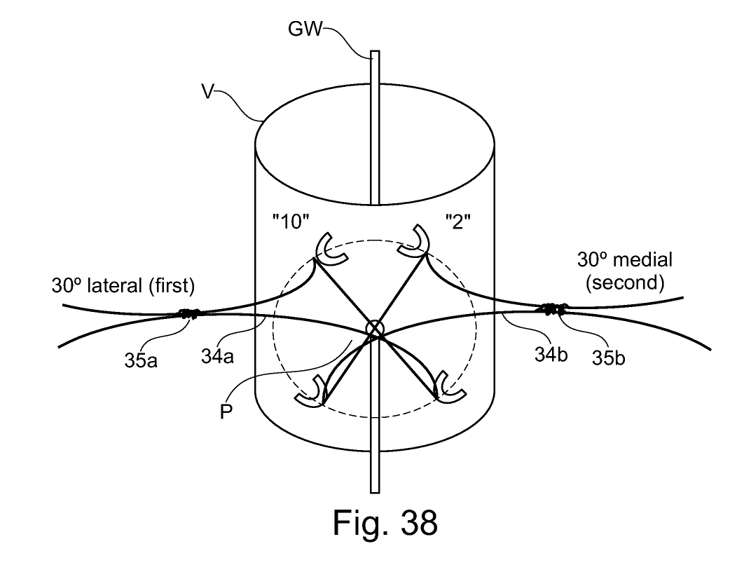

Using the operator's thumb as a fulcrum on the handle, the needles can be disengaged by pulling the plunger assembly back and completely removing the plunger and needles from the body of the device. One suture limb will be attached to the anterior needle. The posterior needle will be free of suture. The operator can then pull back on the plunger until the suture is taut, which confirms that the suture has been fully refracted from the body of the device. (See, e.g., FIGS. 15D-F). The suture is positioned through the vessel wall at 30 degrees towards the patient's right side (approximately 10 o'clock) as shown in FIG. 38.

The suture-trimming mechanism located on the handle of the device can be used to cut the suture from the anterior needle distal of the link. Use of a new, sterile scalpel or scissors can be used alternatively.

The device can be relaxed, and the foot is returned to its original position by pushing the lever on top of the device, down to its original position. (See, e.g., FIGS. 13F and 15F). The lever is closed before removing the device from the patient.

The first vessel closure device is withdrawn from the patient until the guidewire port exits the skin line. The device is slightly rotated until the operator can see the two suture limbs in the bend of the distal guide. The sutures are then grasped adjacent to the sheath. While holding the two suture limbs together, both suture ends are pulled through the distal end of the proximal guide.

A shodded hemostat or clamp can be placed to hold the two suture limbs together at the distal end of the non-rail suture limb (shortest limb). To prevent knot advancement or locking of the knot, care must be taken not to pull on the individual sutures until the clamp is securely holding the two suture limbs together.

The clamp is then gently pulled until the suture is taut to remove any suture slack from the tissue tract. The clamped suture is placed on the right side of the patient under a sterile towel. It is important to identify which suture is deployed first, as this is the knot that needs to be advanced first at the end of the procedure.

The guidewire GW is the reinserted. There should be sufficient guidewire inside the vessel and exposed out of the guidewire exit port for device exchange.

The first vessel closure device is removed, while holding compression above the puncture site and maintaining an adequate length of guidewire inside the artery. This allows placement of a second vessel closure device.

The steps described above are then repeated with the second vessel closure device (i.e., the step of loading the second vessel closure device and its introducer sheath over the guidewire through the step of placing a shodded hemostat or clamp to hold the suture limbs together). As discussed above, the first device was rotated approximately 30 degrees towards the patient's right side (approximately 10 o'clock), the second vessel closure device should be rotated approximately 30 degrees towards the patient's left side (approximately 2 o'clock). As illustrated in FIG. 38, placement of sutures 34a and 34b for right common femoral artery access is shown. Placement of the first device with rotation 30 degrees to patient's left (2 o'clock) results in the placement of sutures 34a and placement of the second device with rotation 30 degrees to patient's right (10 o'clock) results in the placement of sutures 34b.

The operator then removes excess suture slack from the tissue track, and places the clamped suture for the second device on the left side of the patient under a sterile towel. It is important to identify which suture was placed first and which suture was placed second. At the completion of the procedure, the suture knots will be advanced in the order they were placed. The knot 35a from the first device placed on the right side (10 o'clock) of the patient would be advanced, followed by the knot 35b from the second device placed on the left side (2 o'clock) of the patient. Knot advancement will be placed on hold at this point, and the sutures set aside under sterile towels while the operator proceeds with the procedure.

When utilizing a pre-close technique, the vessel closure device is exchanged for an appropriately sized introducer sheath. A medical device such as a stent graft or replacement heart valve is inserted into the vessel to the site of the AAA prior to securing the sutures to close the access point, as discussed below.

After completing the procedure, a hydrophilic or general purpose guidewire is advanced into the artery. An adequate length of guidewire is maintained in both the vessel and exposed out the guidewire exit port to ensure guidewire access is maintained until hemostasis is achieved. The secured sutures are then irrigated with heparinized saline to remove any dry blood. The clamp is removed from the first suture (patient's right side (10 o'clock)).

The rail portion or limb of the suture is the longer of the two suture portions or limbs. This rail suture limb is used to advance the knot. The shorter, non-rail suture limb is used to lock the knot. The rail suture limb securely wrapped around the operator's left forefinger, low close to skin level, and the suture is held coaxial to the tissue tract. While maintaining guidewire access, the entire sheath system is removed and the rail limb is simultaneously pulled with slow, consistent increasing tension. Manual pressure is applied proximal to the puncture site for hemostasis, while the sheath is removed and during initial suture advancement.

The knot is not locked at this point. Due to the size of the arteriotomy, use of the snared knot pusher or suture trimmer discussed above may be needed to approximate the tissue edges. However, the knot is not locked or excessively tightened while the guidewire is still in the vessel. The suture limbs of the first suture are placed on the right side of the patient for easy identification as the first suture deployed.

The clamp is removed from the second suture (patient's left side (2 o'clock)) and the knot is advanced using the same technique, maintaining guidewire access. Again, the know of the second suture is not locked. The suture limbs of the second suture are placed on the left side of the patient for easy identification as the second suture.

The site is then assessed for hemostasis. If brisk bleeding is observed, the first suture (patient's right side (10 o'clock)) is advanced again, and the second suture (patient's left side (2 o'clock)) is advanced again. Multiple knot advancements can be performed when closing larger sheath sizes. However, the knot is not locked or excessively tightened while the guidewire is still in the vessel. Until the wire is removed, some bleeding may be visible, but it should not be pulsatile blood flow.

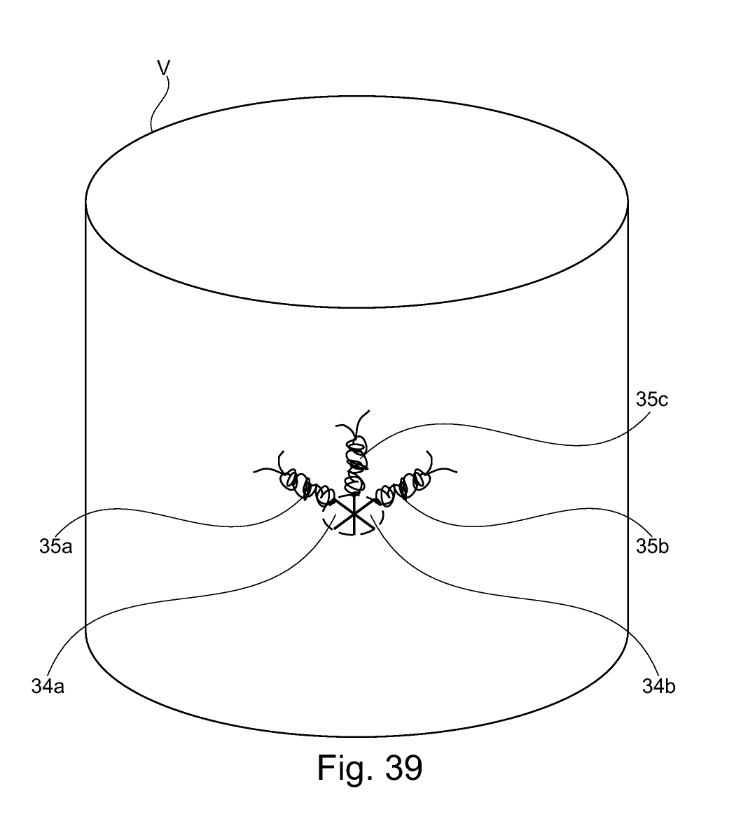

If acceptable hemostasis is not observed, additional vessel closure devices can be deployed at this point. The steps above are repeated (i.e., the step of backloading the device over the guidewire through the step of withdrawing the device until the guidewire port exits the skin line) with the next vessel closure device. However, in some embodiments, the third device is not rotated. Instead, the device will be deployed in a straight cranial/caudad position (12 o'clock) in order to deploy suture 34c as illustrated in FIG. 39. After deployment of this third device, the knot is advanced in the same fashion as discussed herein regarding the first and second suture knots. Again, the knot is not locked or excessively tightened while the guidewire remains in the vessel.

The site is assessed again for adequate hemostasis. If bleeding is controlled, the operator should then remove the guidewire. With the rail suture limb (longer limb) securely wrapped around the left forefinger of the operator, the first suture (patient's right side (10 o'clock)) is advanced, and then the suture trimmer or snared knot pusher is placed under the left thumb to assume a single-handed position and complete knot advancement with slow, consistent increasing tension until the suture is taut (guitar string tightness). With the suture trimmer or snared knot pusher in place and the suture taut, the knot is tightened by gently pulling the non-rail (shorter) suture limb keeping it coaxial to the tissue tract.

The suture is not yet cut. The same steps are followed to advance the second suture (patient's left side (2 o'clock)), coaxial to the tissue tract and lock the knot, but the suture is not cut. If applicable, the additional sutures are advanced, and the knots are locked in the order that they were placed (e.g., 10 o'clock, 2 o'clock, 12 o'clock), and bleeding is assessed. If hemostasis is deemed adequate, then the suture tails are cut below the surface of the skin using the suture trimmer, discussed above, or a new, sterile scalpel or scissors.

Apply an appropriate dressing to the access site. Assess the access site as per hospital standard of care. Patients who have undergone a diagnostic or interventional procedure using 5-8 F sheaths may be ambulated two hours after the vessel closure device procedures. For patients who have undergone an interventional catheterization procedure using 8.5-21 F sheaths, post-procedure ambulation and patient care is at the discretion of the physician. In determining whether to ambulate or discharge an individual patient, all clinical factors are considered, including but not limited to, anticoagulation regimen, antiplatelet and thrombolytic agents administered, oozing or bleeding from the access site, venous access site hemostasis, the general cardiovascular condition of the patient, anesthetic levels, and the overall clinical condition of the patient.

In one embodiment, the closure devices 10, 10′, 100, 300, and 400 described herein are used for closure of a patient's vein.

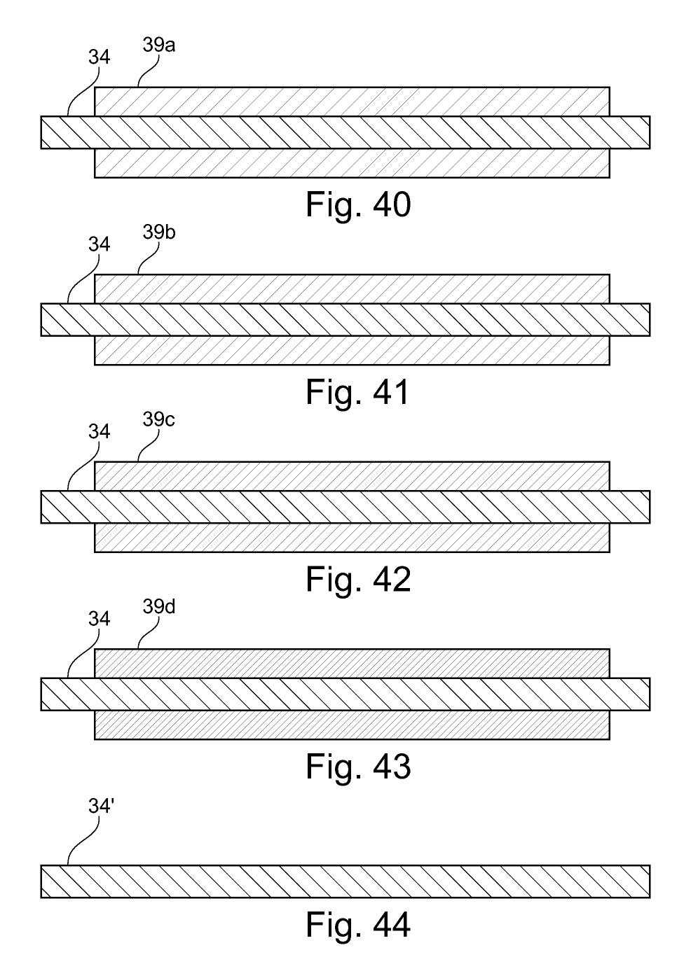

In another embodiment, the closure devices described herein include a coated suture. In one embodiment, the suture 34 is coated with a hydrogel 39a. (FIG. 40.) Hydrogels can promote adhesion of tissue and are biocompatible with the tissue being joined. In another embodiment, the suture 34 is coated with an antibiotic 39b. (FIG. 41.) In a further embodiment, the suture is coated with an anti-inflammatory medication 39c. (FIG. 42.) In a further embodiment, the closure devices described herein include a suture 34 coated with a progoagulent such as protamine sulfate 39d to reverse the anticoagulant effects of heparin. (FIG. 43.)

In another embodiment, the closure devices described include a collagen suture 34′. Such suture is resorbable by the body. (FIG. 44.)

As described hereinabove and illustrated in FIGS. 5 and 29A-B, two or more needles are deployed from the closure device to pierce the wall of the vessel at two or more locations. Such needles carry and position a suture through the wall of the vessel. Such sutures are subsequently withdrawn to close the vessel opening. During deployment, the needles can be radially outwardly (laterally) deflected in order to achieve the desired spread, e.g., such that the separation of the two or more needles spans the opening created in the vessel. For example, as illustrated in FIG. 5, needles 38 are laterally deflected by needle guides 54 of shaft 12. Similarly, FIGS. 29A-B illustrate that guide tip 406 deflects the tips 412 of needles 410 radially outward as the needles 410 are refracted within the guide tip 406.

In a further embodiment, the closure devices described herein are provided with the feature of selectively adjustable spacing of the needles for positioning sutures through a wall of a vessel in connection with the closure of vessel openings which vary in size, e.g., from about 8 Fr to about 24 Fr. This feature permits the surgeon to select the “spread” of the needles, e.g., the distance between the needle tips, during the procedure, as required by the size of the particular vessel opening being closed. For example, it is desirable in certain procedures to produce an opening in the vessel of about 8 Fr. In other procedures, the opening in the vessel may be as large as 24 Fr in order to accommodate larger apparatus, such as expandable grafts. Thus, vessel openings of different sizes can be closed with the use of single vessel closure device.

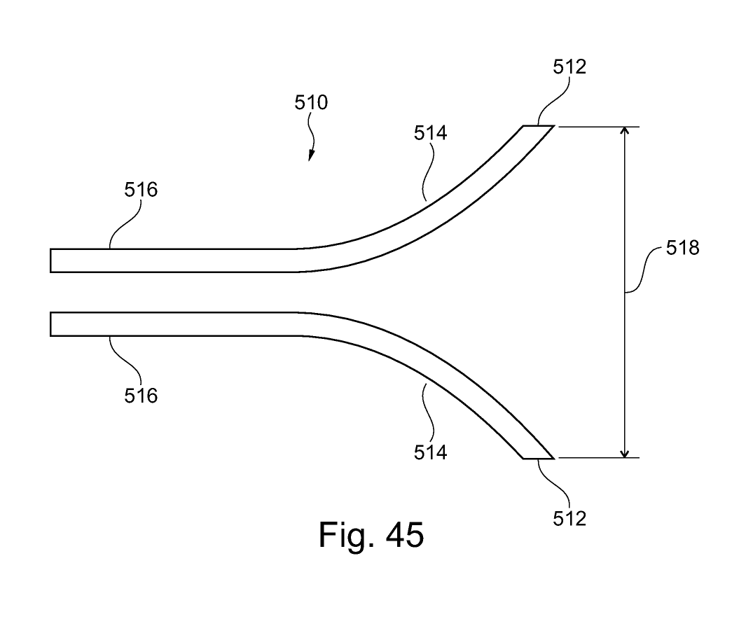

As illustrated in FIGS. 45-48, a needle deployment portion of a vessel closure device 500 is provided. In some embodiments, vessel closure device 500 is used in a percutaneous method of treating a blood vessel. An access point in a blood vessel of a patient proximal to the affected site is achieved without a surgical cut-down procedure, as described above. A guidewire is advanced through the access point to a location distal to the affected site. The closure device is advanced into the blood vessel, and a suture is positioned through a wall of the blood vessel across the access point. The sheath is sized to fit through an opening in the blood vessel of about 8 Fr to about 24 Fr. In order to enter the vessel and traverse portion of the vessel, at least a portion of the shaft and the sheath are flexible.

In some embodiments, the needles 510 are fabricated from a material having flexible and/or resilient characteristics, and can be fabricated from a shape memory alloy, such as nitinol. As illustrated in FIG. 45, needles 510 have tips 512, a flexible portion 514 having a pre-curved shape in its relaxed state, and a rear portion 516, which may be straight in some embodiments. Sutures can be attached to rear portion 516 (not shown). When two such needles are placed side-by-side, as shown in the figure, the tips 512 of the needles 510 span a distance 518. The flexible portions 514 of the needles 510 can be restrained within a sheath. An exemplary embodiment of an apparatus 500 is illustrated in FIG. 45A. The needles 510 are positioned within sheath 502. The sheath 502 includes a collar 555 at the proximal end for manipulation by the surgeon. The proximal portion 516 of the needles 510 are secured to an actuator 557. (Actuator 557 includes a bore 558 to allow additional optional instrumentation to pass therethrough.) The surgeon can selectively vary the spread of the needles by relative longitudinal movement of the sheath with respect to the needles. In the exemplary embodiment, such relative longitudinal movement is achieved by the movement of the actuator 557 with respect to the collar 555. When all or a significant portion of the needles are restrained, the tips 512 of the needles span a smaller distance. As a greater portion of the needles is exposed, the angle of the needle changes, and a greater portion of flexible portions 514 are able to return to their pre-curved shape to attain a larger spacing of the tips 512.

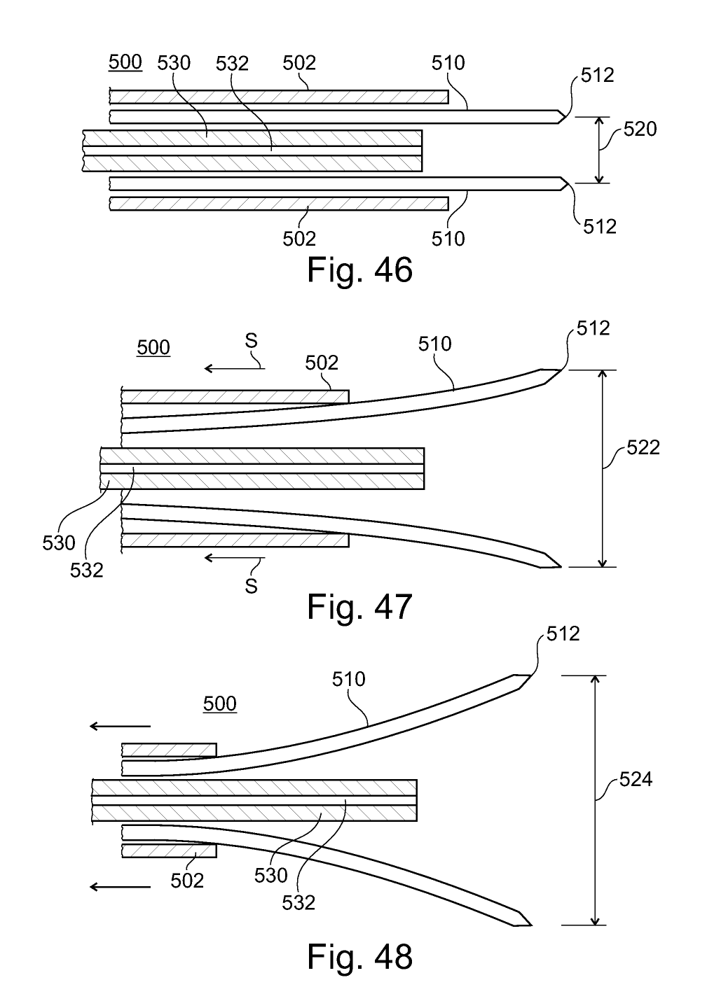

FIGS. 46-48 illustrate a portion of closure device 500, provided with a sheath 502 and a plurality of needles 510 having tips 512 for piercing the vessels being closed. As illustrated in FIG. 46, the needles 510 are at least partially constrained within the sheath 502 and have an initial spread 520. Device 500 can further include a shaft 530 defining a central bore 532 for a guidewire to pass therethrough.

FIG. 47 illustrates the sheath 502 is partially withdrawn in a direction opposite from the tips 512 of the needle 510. Relative sheath movement is illustrated by arrow S. As the sheath 502 is withdrawn, needles 510 are permitted to flexibly return to a pre-curved configuration. For example, by withdrawing the sheath 502 a first distance from the position shown in FIG. 46 to the position show in FIG. 47, the needles 510 are permitted to displace laterally to a spread 522. In some embodiments, spread 522 is about 8 Fr.

FIG. 48 illustrates that the sheath 502 is further withdrawn in a direction opposite from the tips 512 of the needle 510. Further movement of sheath 502 allows the needles 510 to further displace laterally to a spread 524. In some embodiments, spread 524 is about 24 Fr. As a consequence of using the embodiment of vessel closure device 500, a single device may be used to close openings of different sizes. The rear portions 516 of the needles 510 can be attached to a suture. After needles 510 pass through the wall of the vessel, the needles carry the suture through the wall of the vessel, thereby positioning the suture across the access point. Afterwards, the closure device is removed from the subject. A sheath, e.g., 8 F or larger, can be introduced over the guidewire to the affected area, and a medical device is delivered to the location of the affected site. The suture is secured to close the access point of the blood vessel.

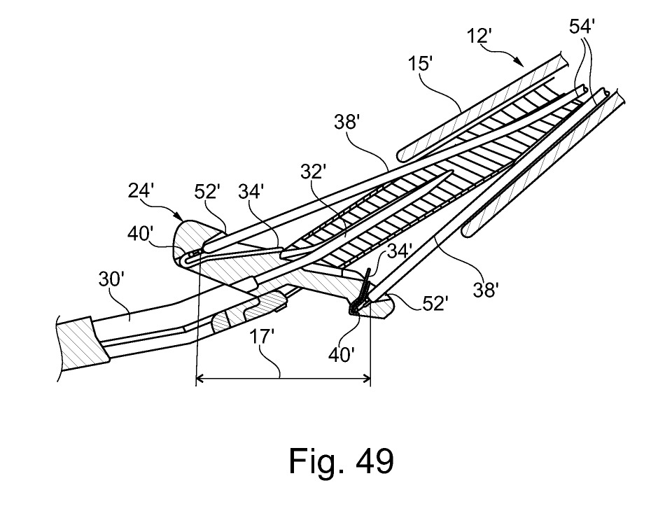

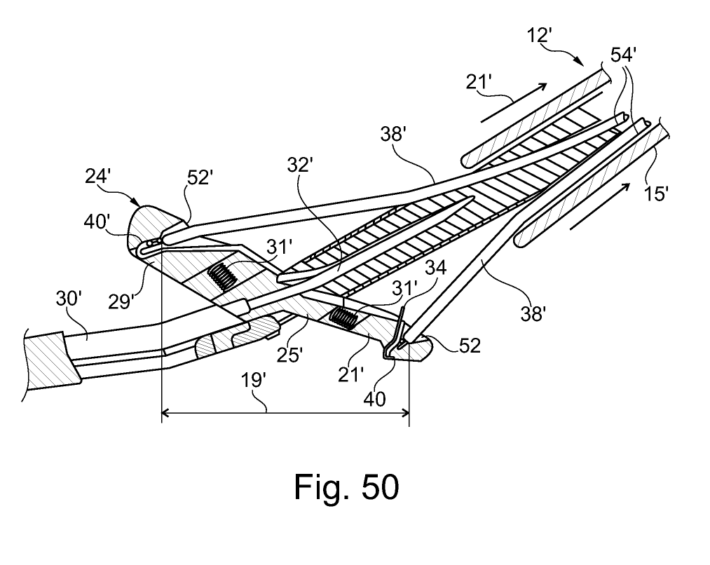

Without limiting the above disclosure, the mechanism illustrated in FIGS. 46-48 can be incorporated into the exemplary devices of FIGS. 5 and 29A-B. For example, FIGS. 49 and 50 illustrate an exemplary embodiment of a vessel closure device that is substantially identical to closure device 10 described hereinabove, with the substantial differences noted herein. For example, the vessel closure device depicted in FIGS. 49 and 50 includes selective adjustable spacing of the needles. In the exemplary embodiment, needles 38′ extend from shaft 12′ into secured engagement with fittings 40′ attached to suture(s) 34′. Needles 38′ are fabricated with a flexible and/or shape memory feature as described above regarding needle 510. Shaft 12′ further includes a sheath 15′, that is substantially identical to sheath 502 discussed above. As sheath 15′ is refracted proximally (towards the operator, and away from the needle tips), the spread of the needles 38′ can be adjusted. FIG. 49 illustrates spread 17′ between needles 38′, corresponding to the position of sheath 15′ shown in the Figure. FIG. 50 illustrates a greater spread 19′ between needles 38′, corresponding to the position of sheath 15′ shown in the Figure. Sheath 15′ has been further retracted from needles 38′ in the direction of arrow 21′, thereby allowing the needles 38′ to deflect laterally to a greater extent.

As was generally described above, foot 24′ includes needle receptacles 52′ adjacent the ends of the foot. A fitting 40′ (with an associated end of suture 34′) is disposed within each needle receptacle, and a surface of the receptacle tapers proximally and outwardly so as to guide the advancing needles 38′ into engagement with fittings 40′ when foot 24′ is in the deployed position. As fittings 40′ (and associated portions of suture 34′) are releasably supported in the foot, needles 38′ can be withdrawn proximally so as to draw the fittings and suture ends from the foot proximally into (and optionally through) shaft 12′.

In some embodiments, foot 24′ includes features in order to accommodate the variable spread of the needles 38′. For example, foot 24′ can incorporate a center component 25′ and two outer components 27′ and 29′ which are slidable (or otherwise movable) with respect to the center component 25′ via springs 31.′ In this manner, outer components 27′ and 29′ (and corresponding receptacles 52′) can be maintained in a first position as shown in FIG. 49 when the needles 38′ have spread 17′ and in a second position as shown in FIG. 50 when the needles 38′ have the greater spread 19′. It is understood that varying the spacing of receptacles 52′ (and components 27′ and 29′) may be linked to the withdrawal of the sheath 15′ by a mechanical linkage.

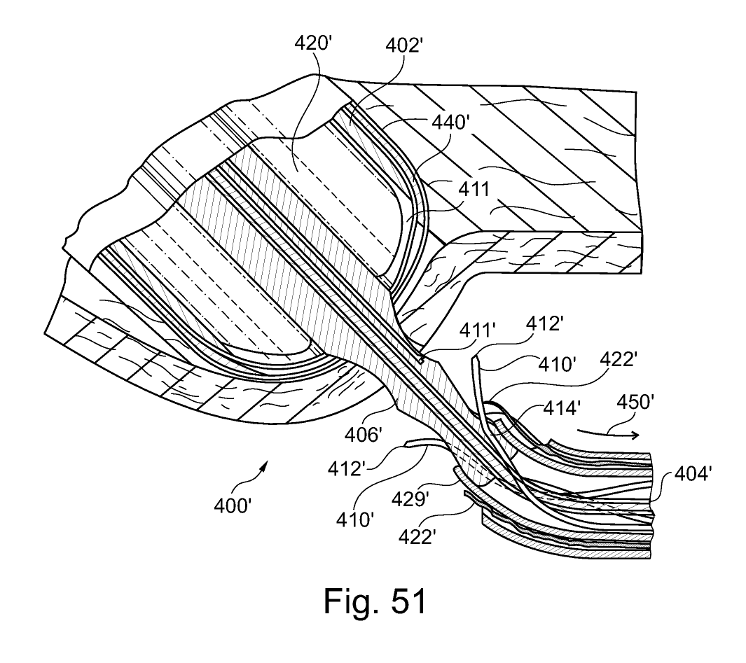

FIG. 51 illustrates a further embodiment of the vessel closure device 400′ which incorporates selective adjustable spacing of the needles. The exemplary embodiment illustrated in FIG. 51 is substantially identical to the suture applying device 400 illustrated in FIGS. 29A-B, which the significant differences noted herein.

The device 400′ comprises a guide body 402′ and a needle shaft 404′. The guide body 402′ includes a guide tip 406′ at its distal end, which guide tip includes a plurality of guide channels to receive the proximal ends of needles 410′. The needles 410′ as illustrated comprise a sharpened tip section 412′ and an elongate shank portion 414′, but may also be manufactured as an integral piece. Needles 410′ are fabricated in the same manner as needle 510 and 38′ discussed above, e.g., having a pre-formed bent configuration to allow for a selectively adjustable spread between the needle tips 412′. The shank portion 414′ will be sufficiently long so that the needles may be pushed from their butt end by a support holster 428 (illustrated in FIGS. 31-32) fixedly attached to the needle shaft 404′ in order to advance the needles through the tissue to be sutured and fully through the guide body 402′ inserted together with support sheath 440′ in the associated tract so that no capture mechanism will be required.

The guide body 402 further includes a plurality of needle lumens 420′ which are axially aligned and spaced about the periphery of the guide body. The needles 410′ will enter the distal ends of the lumens 420′ as the needles are advanced proximally relative to the guide body.

A flexible needle sheath 429′ is slidable over the needle shaft 404′. Prior to use, the sheath 429′ will cover a first portion of the needles 410′. That is, the needles will define a first spread configuration. As the sheath is retracted from the needles (in the direction illustrated by arrows denoted 450′), the needles are permitted to resume their pre-bent configuration, thereby defining a greater spread between needle tip 412′ as illustrated in FIG. 51. As illustrated in FIG. 49, the 402′ can be provided with a larger diameter in order to receive the needle tip 412′ defining the greater spread discussed above.

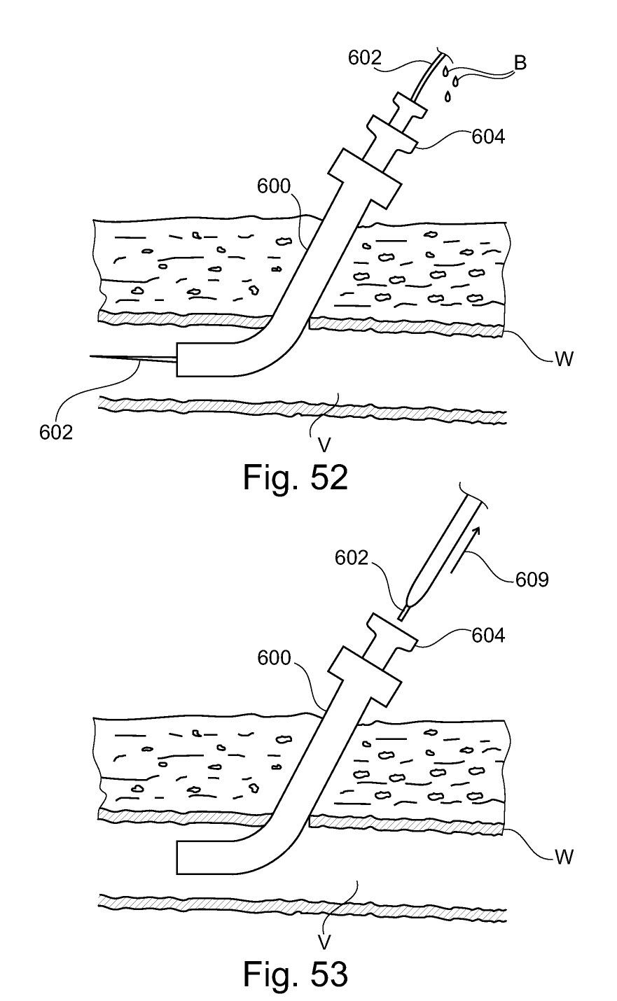

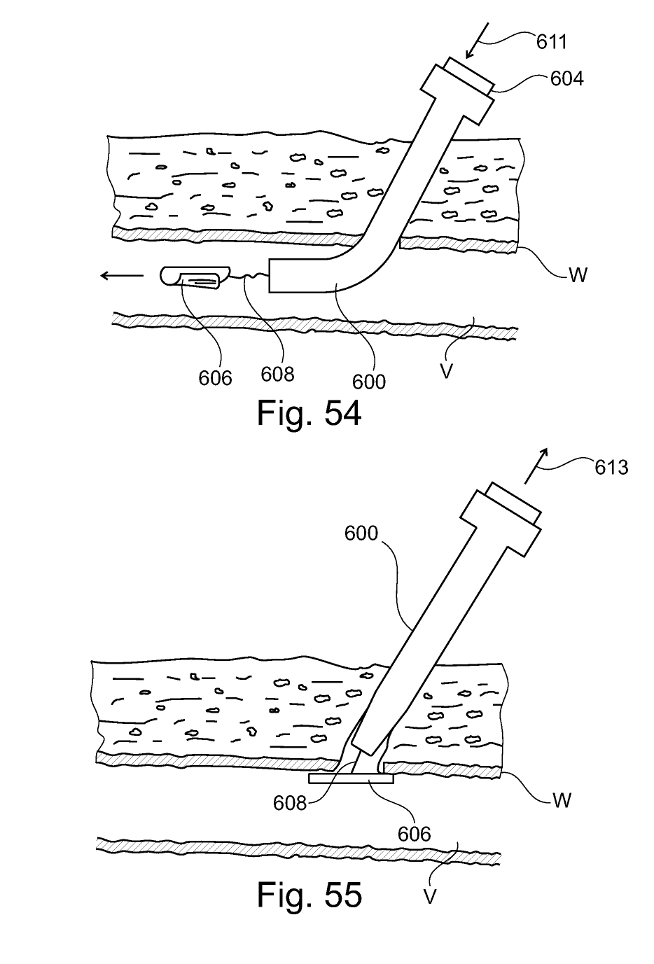

FIGS. 52-59 illustrate an exemplary embodiment for a vessel closure device used in a totally percutaneous method of treating a blood vessel. An access point in a blood vessel of a patient proximal to the affected site is achieved without a surgical cut-down procedure, as described above. Such device may be used in conjunction with the vessel closure devices described herein or independently of such devices. FIG. 52 illustrates that the guidewire 602 is advanced at an access point by passing through the wall W of the vessel V. The closure device is advanced into the vessel V. An introducer, e.g., catheter 600, is deployed over the guidewire 602. An indicator lumen can be provided (not shown) which permits the flow of blood (droplets B) which serves to indicate that the distal end of the catheter 600 has entered the vessel V. As illustrated in FIG. 52, the guidewire 602 is removed from the operative site in the proximal direction (arrow 609).

FIG. 53 illustrates the distal advancement of ejection tube 604 (arrow 611) into catheter 600. By advancing the ejection tube 604, the anchor 606, secured to a suture 608, is advanced into the vessel V. In some embodiments, the anchor 606 has a semi-cylindrical contour to conform to the wall of the vessel. The anchor 606 is fabricated from materials such as biocompatible plastics, metals, etc. Subsequently, the catheter 600, as well as the ejection tube 604, are withdrawn from the vessel V, as illustrated in FIG. 55. (Arrow 613). Consequently, the anchor 606 is drawn into contact with the wall W of the vessel V.

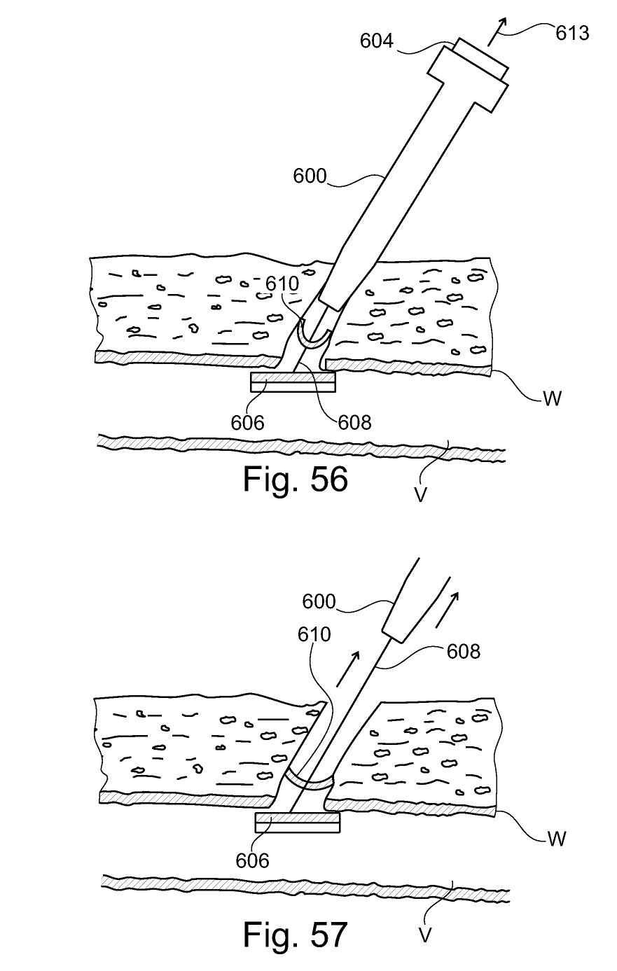

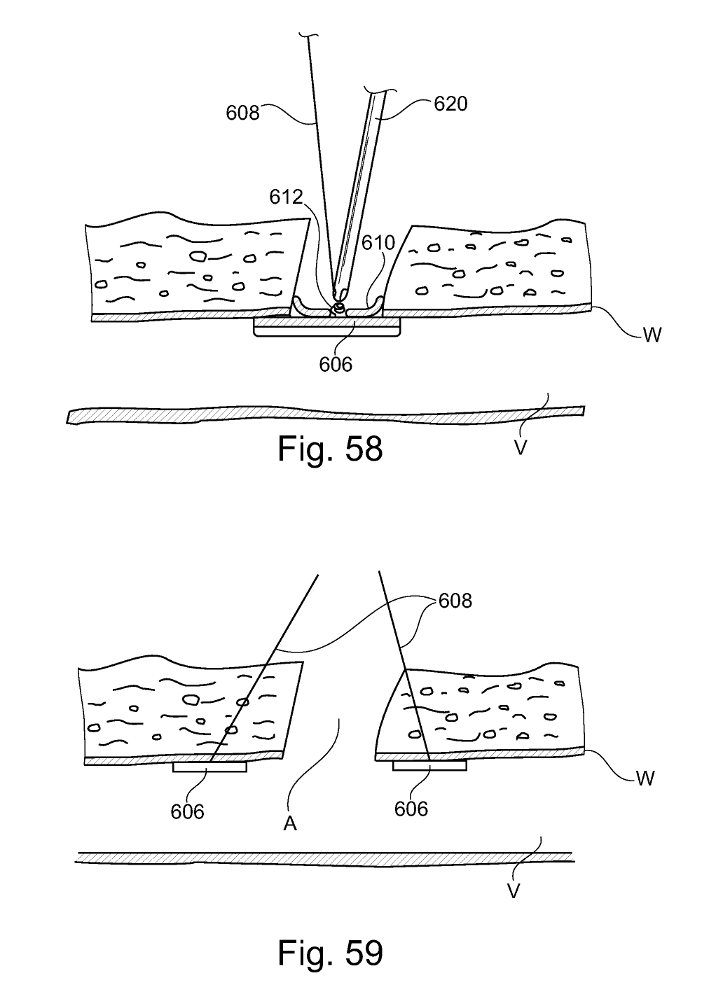

As illustrated in FIG. 56, further tension on suture 608 caused by retraction of catheter 600 and ejection tube 604 from the vessel V (arrow 613) causes the deployment of cap 610 from the distal end of the catheter 600. The cap 610 includes an aperture that is slidable over the suture 608. In some embodiments cap 610 is slidable on the suture 608. Cap 610 can be flexible and fabricated from materials such as biocompatible plastics and metals. FIG. 57 illustrates removal of the catheter 600 from the vessel, leaving the cap 610 positioned within the opening in the vessel V. FIG. 58 illustrates that the cap 610 is seated within the opening, thereby securing the anchor 606 in place. For example, a cleat or knot 612 can be formed from suture 608, which is secured by a suture trimmer or knot pusher 620. In a procedure, two or more anchors 606 are deployed in tissue and sutures 608 are tightened in order to close the vessel opening A. (FIG. 59).

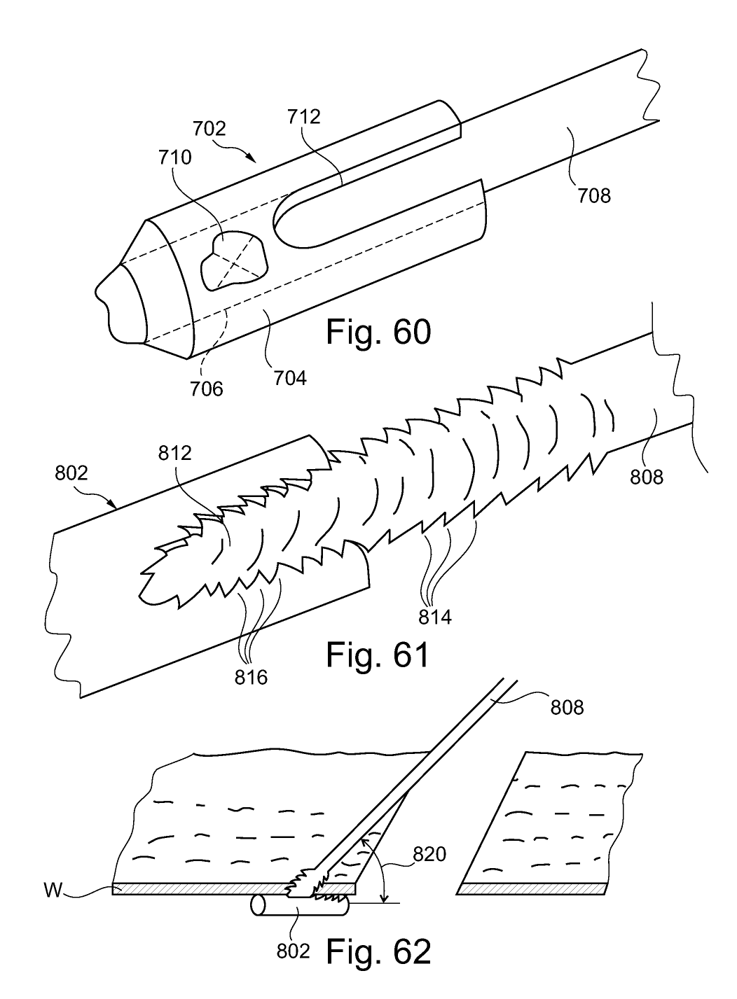

A further embodiment of an anchor for securing a suture deployed, e.g., through a hypotube or catheter, is illustrated in FIG. 60. An exemplary procedure for deploying anchor 702 is illustrated in FIGS. 52-59, above. Anchor 702 can be fabricated from magnesium in certain embodiments. As depicted in FIG. 60, anchor 702 has a substantially cylindrical body 704 including a bore or central cavity 706 (indicated in dashed line) for receiving a suture 708, such as a bioabsorbable suture. The suture 708 is retained in the anchor 702 by, e.g., mechanical crimping 710 of the anchor 702. A notch 712 is provided in the body of the anchor 702 which allows the suture to be positioned at an angle to the anchor. (See FIG. 62, below.)

In another embodiment illustrated in FIG. 61, anchor 802 and suture 808 arrangement is deployed through an introducer, e.g., a hypotube or catheter, and is substantially identical to the anchor 702 and suture 708 described above. In addition, stability of the position of anchor 802 against the wall W of the vessel is provided by vanes 814 cut into the suture 808. Vanes 814 allow tensioning of the anchor 802 against the wall W of the vessel and resist slippage through the hypo tube access tract. Ridges 816 are provided in the anchor 802 to further improve anchor stability. FIG. 62 illustrates the positioning of the anchor 802 within the vessel. The notch 812 in the body 804 of the anchor 802 allows the suture 808 to be disposed at an angle 820 to the longitudinal axis of the anchor 802. The vanes 814 provide stability of the suture 808, and the ridges 816 provide stability of the anchor.

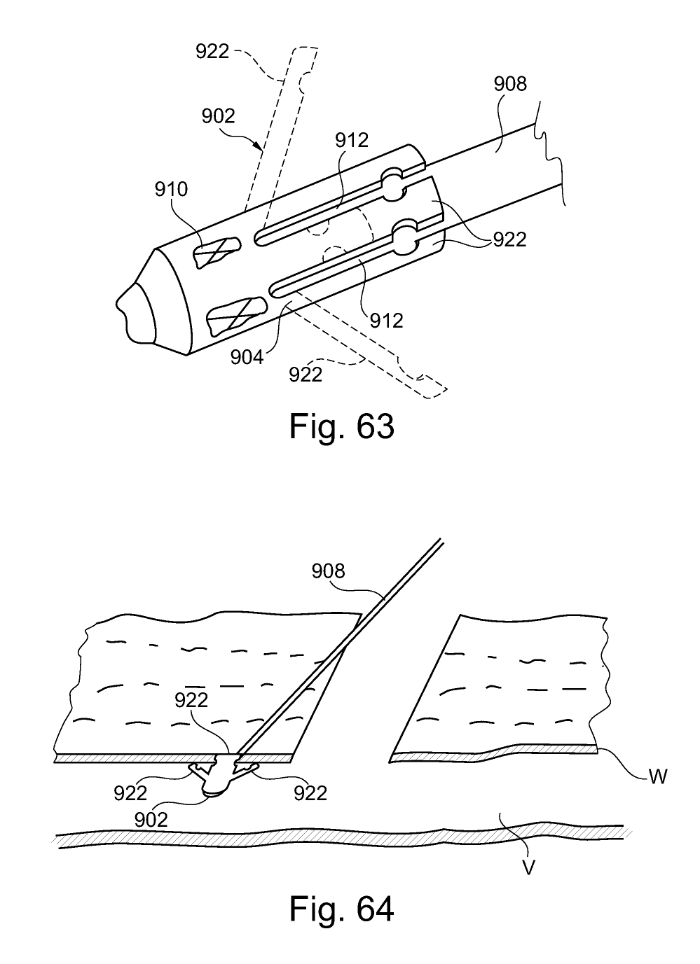

A further embodiment of an anchor for securing a suture deployed through an introducer, e.g., a hypotube or catheter, is illustrated in FIG. 63. Anchor 902 can be fabricated from magnesium in certain embodiments. As depicted in FIG. 63, anchor 902 has a substantially cylindrical body 904 including a bore or central cavity for receiving a suture 908, such as a bioabsorbable suture. The suture 908 is retained in the anchor 902 by, e.g., mechanical crimping 910 of the anchor 902. A plurality of longitudinal notches 912 are provided in the body of the anchor 902, forming a plurality of grapple legs 922. Grapple legs 922 are designed to deflect radially outwardly, as indicated in dashed line in FIG. 63. When installed in the vessel V, the grapple legs 922 engage the interior of the wall W of the vessel V, thereby securing the suture 908 in place. (FIG. 64.)

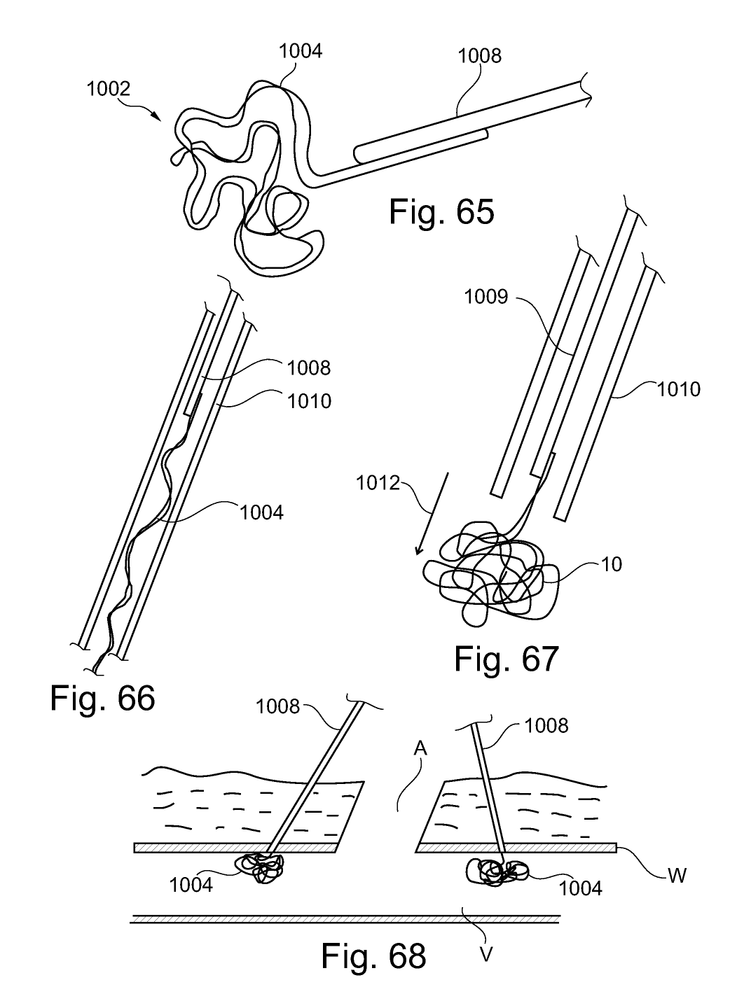

According to a further embodiment illustrated in FIG. 65, an anchor 1002 with the suture 1008 attached is deployed through an introducer, e.g., a hypotube or catheter, after the hypotube has penetrated the wall W of the vessel V. According to this embodiment, the anchor 1002 is formed by a resilient strip formed into a coil or twisted ribbon 1004. (FIG. 65). One embodiment of the coil/ribbon 1004 includes the shape memory properties of nitinol. The coil 1004 can be formed into a plume of spherical shape of about 2-3 times the diameter of the introducer hole, e.g., the opening in the tissue made by the hypotube 1010. Alternatively, the coil 1004 should be at least sufficiently large to withstand resistance when the suture 1008 is pulled against it to avoid passing through the pierced vessel wall. The coil can be created by inserting the wire into a container approximately the same size and shape as the intended coil. The coil can be attached to the suture by adhesive, pressure or heat treatment, etc.

Because of the shape memory properties, the resilient strip 1004 can be straightened and stored inside a lumen of the hypotube 1010 (FIG. 66), and then inserted through the wall W of the vessel. The ribbon 1004 is advanced from the hypotube 1010 (arrow 1012), or alternatively the hypotube 1010 is withdrawn, thereby allowing the nitinol strip or wire 1004 to resume its final shape of a coil (e.g., ball or plume). (FIGS. 67 and 68.) Upon tension by the suture 1008, the coil 1004 is sufficiently tight to be able to retain the coiled shape and withstand resistance to return to the straightened configuration. In some embodiments, nicks in the ribbon or wire can be created in order to maintain the coil configuration. As illustrated in FIG. 68, two coils 1004 and their associated sutures 1008 are positioned adjacent and access site, such as an arteriotomy. After medical procedures are performed, e.g., introducing a sheath to the affected area and/or delivering a medical device, the sutures 1008 are secured to close the access point of the vessel.

While particular embodiments of the invention have been herein described in detail, it is to be appreciated that the present invention encompasses variations and combinations thereof, as may be apparent to one of ordinary skill from this disclosure. It is therefore to be understood that within the scope of the appended claims, the invention may be practiced otherwise than as specifically described herein.

What is claimed is:

1. A totally percutaneous method of treating an affected region of a blood vessel, the method comprising:
creating an access point in a blood vessel of a patient proximal to the affected site without a surgical cut-down procedure;
advancing a guidewire through the access point and traversing the guidewire to a location distal to the affected site;
advancing a closure device into the blood vessel;
selectively adjusting the spacing of a plurality of needles;
advancing the needles through a wall of the blood vessel wall thereby positioning a suture through the wall of the blood vessel across the access point;
removing the closure device from the subject; and
securing the suture to close the access point of the blood vessel.

2. The method according to claim 1, further comprising providing two or more needles having a first pre-curved configuration and a second straightened configuration, and wherein selectively adjusting the spacing of the needles comprises withdrawing a sheath surrounding the needles thereby permitting the needles to move from the second configuration to the first configuration.

3. The method according to claim 1, wherein the spacing of the plurality of needles is adjustable between about 8 Fr and about 24 Fr.

4. The method according to claim 1, wherein the blood vessel is a vein.

5. The method according to claim 1, wherein the suture is coated with a hydrogel.

6. The method according to claim 1, wherein the suture is coated with an antibiotic.

7. The method according to claim 1, wherein the suture is coated with a pro-coagulent medication.

8. The method according to claim 1, wherein the suture is coated with an anti-inflammatory medication.

9. The method according to claim 1, where advancing the closure device into the blood vessel comprises advancing the closure device at an angle of about 45° relative to the blood vessel.

10. The method according to claim 1, further including dilating the blood vessel prior to insertion of the closure device.

11. The method according to claim 1, wherein the suture is a monofilament suture.

12. The method according to claim 1, wherein positioning a suture through the wall of the blood vessel across the access point comprises positioning the suture through the wall of the blood vessel at first angle with respect to the longitudinal axis of the patient; and further comprising
after removing the first closure device from the subject, advancing a second closure device into the blood vessel;
positioning a second suture through a wall of the blood vessel across the access point at a second angle with respect to the longitudinal axis of the patient; and
removing the second closure device from the subject.

13. The method according to claim 12, wherein the first angle is 60 degrees in a first direction from the longitudinal axis of the patient.

14. The method according to claim 13, wherein the second angle is 60 degrees in a second direction from the longitudinal axis of the patient.

15. A totally percutaneous method of treating an affected region of a blood vessel, the method comprising:
creating an access point in a blood vessel of a patient proximal to the affected site without a surgical cut-down procedure;
advancing a guidewire through the access point and traversing the guidewire to a location distal to the affected site;
advancing a closure device into the blood vessel;
positioning an anchor carrying a suture therewith through a wall of the blood vessel across the access point;
deploying the anchor with respect to the wall of the blood vessel;
removing the first closure device from the subject; and
securing the suture to close the access point of the blood vessel.

16. The method according to claim 15, wherein the blood vessel is a vein.

17. The method according to claim 15, further comprising providing an anchor defining a longitudinal axis, a central opening for retaining a suture therein, and a notch extending from the central opening allowing the suture to pass from the central opening through the notch at an angle with respect to the longitudinal axis, and wherein deploying the anchor comprises withdrawing the suture from the vessel such that the anchor engages the wall of the vessel and the suture extends from the anchor at a angle with respect to the longitudinal axis of the anchor.

18. The method according to claim 17, further comprising defining a plurality of vanes on the suture, and wherein deploying the anchor comprises engaging the vanes with the notch of the anchor and the vessel wall.

19. The method according to claim 17, further comprising defining a plurality of ridges on the notch for engaging the suture, and wherein deploying the anchor comprises engaging the suture with the ridges of the notch.

20. The method according to claim 17, wherein the anchor is fabricated from magnesium.

21. The method according to claim 15, further comprising further comprising providing an anchor defining a longitudinal axis, a central opening for retaining a suture therein, and a plurality of longitudinal notches defining a plurality of grappler arms, and wherein deploying the anchor comprises withdrawing the suture from the vessel such that the plurality of grappler arms are displaced radially outwardly from the longitudinal axis and engage the wall of the vessel.

22. The method according to claim 15, further comprising providing an anchor comprising a resilient strip having a first configuration defining a coil and a second straightened configuration, and wherein advancing a closure device into the blood vessel comprises restraining the resilient strip in the first configuration within an introducer.

23. The method according to claim 22, wherein positioning the anchor carrying a suture therewith through a wall of the blood vessel across the access point comprises advancing the introducer through the wall of the blood vessel with the resilient strip disposed therein.

24. The method according to claim 23, wherein deploying the anchor with respect to the wall of the blood vessel comprises advancing the resilient strip with respect to the introducer into the blood vessel, thereby allowing the resilient strip to return to the first configuration.

25. An apparatus for the percutaneous closure of an opening created in a blood vessel comprising:
a shaft defining a bore therethrough for guidewire access;
two or more needles defining a first configuration defining a curved configuration and a second straightened configuration;
a sheath surrounding the needles and advanceable from a first position in which the needles are restrained in the second configuration and a second position in which the needles are permitted to return to the first configuration; and
one or more sutures attached to an end portion of the needles.

26. The apparatus according to claim 25, wherein the sheath is sized to fit through an opening in the blood vessel of about 8 Fr.

27. The apparatus according to claim 25, wherein the sheath is sized to fit through an opening in the blood vessel of about 24 Fr.

28. The apparatus according to claim 25, wherein the needle is fabricated from a shape memory alloy.

29. The apparatus according to claim 28, wherein the needle is fabricated from nitinol.

30. The apparatus according to claim 25, wherein the shaft and the sheath are flexible.

31. An apparatus for the percutaneous closure of an opening created in a blood vessel comprising:
an anchor defining a longitudinal axis, a central opening for retaining a suture therein, and a notch extending from the central opening allowing the suture to pass from the central opening through the notch at an angle with respect to the longitudinal axis;
a suture partially positioned within the central opening and retained therein, the suture partially extending from the central opening; and
an introducer for advancing through the wall of the vessel and accommodating the anchor therein.

32. The apparatus according to claim 31, wherein the suture defines a plurality of vanes thereon for engagement with the notch of the anchor and the vessel wall.

33. The apparatus according to claim 32, wherein the notch defines a plurality of ridges for engaging the suture.

34. The apparatus according to claim 31, wherein the anchor is fabricated from magnesium.

35. An apparatus for the percutaneous closure of an opening created in a blood vessel comprising:
an anchor defining a longitudinal axis, a central opening for retaining a suture therein, and a plurality of longitudinal notches defining a plurality of grappler arms for engagement with the wall of the vessel;
a suture partially positioned within the central opening and retained therein, the suture partially extending from the central opening; and
an introducer for advancing through the wall of the vessel and accommodating the anchor therein.

36. An apparatus for the percutaneous closure of an opening created in a blood vessel comprising:
an anchor comprising a resilient strip having a first configuration defining a coil and a second straightened configuration, and wherein advancing a closure device into the blood vessel comprises restraining the resilient strip in the first configuration within an introducer;
a suture attached to the anchor; and
an introducer for advancing through the wall of the vessel and accommodating the anchor therein in the first configuration and for deploying the anchor therefrom in the second configuration.

37. The apparatus according to claim 36, wherein the resilient strip is fabricated from nitinol.

38. The apparatus according to claim 36, wherein the dimension of the coil is about twice as large as the dimension of the introducer.
